SEQUENCE LISTING (105 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 3 - DNA, 396 bp, Artificial Sequence
  Description of Artificial Sequence Synthetic polynucleotide
  Human IL-15 with GMCSF signal peptide
atgtggctccagagcctgctactcctggggacggtggcctgcagcatctcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtga

SEQ ID NO: 4 - DNA, 1847 bp, Artificial Sequence
  Description of Artificial Sequence Synthetic polynucleotide
  IL-15 codon optimized DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacaagaaatgcggatctcgaagccgcacctgcggtcgatatcgatccagtgctacctgtgcctgctcctgaactcgcacttcctcacggaggccggtatacacgtcttcatcctgggctgcttctcggcggggctgccgaagacggaggcgaactgggtgaacgtgatctcggacctgaagaagatcgaggacctcatccagtcgatgcacatcgacgcgacgctgtacacggagtcggacgtccacccgtcgtgcaaggtcacggcgatgaagtgcttcctcctggagctccaagtcatctcgctcgagtcgggggacgcgtcgatccacgacacggtggagaacctgatcatcctggcgaacaactcgctgtcgtcgaacgggaacgtcacggagtcgggctgcaaggagtgcgaggagctggaggagaagaacatcaaggagttcctgcagtcgttcgtgcacatcgtccagatgttcatcaacacgtcgtgagggcccggcgcgccgaattcgcggatatcggttaacggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 5 - protein, 162 aa, Artificial Sequence; identical to SEQ ID NO: 1
  Description of Artificial Sequence Synthetic polypeptide
  IL-15 codon optimized amino acid

SEQ ID NO: 6 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 200 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 8 - DNA, 804 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 9 - DNA, 600 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggc

SEQ ID NO: 10 - protein, 170 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 11 - DNA, 2140 bp, Artificial Sequence
  Description of Artificial Sequence Synthetic polynucleotide
  IL-15Ra codon optimized DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgctagcaagaaatggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacggtggcgatctccacgtccacggtcctgctgtgtgggctgagcgcggtgtcgctcctggcgtgctacctcaagtcgaggcagactcccccgctggccagcgttgagatggaggccatggaggctctgccggtgacgtgggggaccagcagcagggatgaggacttggagaactgctcgcaccacctataatgagaattcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 12 - protein, 267 aa, Artificial Sequence; identical to SEQ ID NO: 6
  Description of Artificial Sequence Synthetic polypeptide
  IL-15Ra codon optimized amino acid

SEQ ID NO: 13 - DNA, 1971 bp, Artificial Sequence
  Description of Artificial Sequence Synthetic polynucleotide
  CMV IL-15Ra codon optimized DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgacgctagcaagaaatggccccgaggcgggcgcgaggctgccggaccctcggtctcccggcgctgctactgctcctgctgctccggccgccggcgacgcggggcatcacgtgcccgccccccatgtccgtggagcacgcagacatctgggtcaagagctacagcttgtactcccgggagcggtacatctgcaactcgggtttcaagcggaaggccggcacgtccagcctgacggagtgcgtgttgaacaaggccacgaatgtcgcccactggacgaccccctcgctcaagtgcatccgcgacccggccctggttcaccagcggcccgcgccaccctccaccgtaacgacggcgggggtgaccccgcagccggagagcctctccccgtcgggaaaggagcccgccgcgtcgtcgcccagctcgaacaacacggcggccacaactgcagcgatcgtcccgggctcccagctgatgccgtcgaagtcgccgtccacgggaaccacggagatcagcagtcatgagtcctcccacggcaccccctcgcaaacgacggccaagaactgggaactcacggcgtccgcctcccaccagccgccgggggtgtatccgcaaggccacagcgacaccacgtaatgagaattcgcggatatcggttaacggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcata

SEQ ID NO: 14 - protein, 205 aa, Artificial Sequence
  Description of Artificial Sequence Synthetic polypeptide
  CMV IL-15Ra codon optimized amino acid
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 15 - protein, 8 aa, Artificial Sequence
  Description of Artificial Sequence Synthetic peptide
  C-terminal of soluble human IL-15Ra
PQGHSDTT

SEQ ID NO: 16 - protein, 7 aa, Artificial Sequence
  Description of Artificial Sequence Synthetic peptide
  C-terminal of soluble human IL-15Ra
PQGHSDT

SEQ ID NO: 17 - protein, 6 aa, Artificial Sequence
  Description of Artificial Sequence Synthetic peptide
  C-terminal of soluble human IL-15Ra
PQGHSD

SEQ ID NO: 18 - protein, 5 aa, Artificial Sequence
  Description of Artificial Sequence Synthetic peptide
  C-terminal of soluble human IL-15Ra
PQGHS

SEQ ID NO: 19 - protein, 4 aa, Artificial Sequence
  Description of Artificial Sequence Synthetic peptide
  C-terminal of soluble human IL-15Ra
PQGH

SEQ ID NO: 20 - protein, 3 aa, Artificial Sequence
  Description of Artificial Sequence Synthetic peptide
  C-terminal of soluble human IL-15Ra
PQG

SEQ ID NO: 21 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 22 - protein, 19 aa, Artificial Sequence
  Description of Artificial Sequence Synthetic peptide
  IL-15Ra O-glycosylation
  MISC_FEATURE (5)..(5): May or may not be O-glycosylated
  MISC_FEATURE (7)..(7): May or may not be O-glycosylated
  See specification as filed for detailed description of substitutions and preferred embodiments
NWELTASASHQPPGVYPQG

SEQ ID NO: 23 - protein, 17 aa, Artificial Sequence
  Description of Artificial Sequence Synthetic peptide
  IL-15Ra N-glycosylation
  MISC_FEATURE (8)..(8): May or may not be N-glycosylated
  See specification as filed for detailed description of substitutions and preferred embodiments
ITCPPPMSVEHADIWVK

SEQ ID NO: 24 - protein, 31 aa, Artificial Sequence
  Description of Artificial Sequence Synthetic polypeptide
  IL-15Ra N-glycosylation
  MISC_FEATURE (8)..(8): May or may not be N-glycosylated
  MISC_FEATURE (18)..(18): May or may not be N-glycosylated
  MISC_FEATURE (20)..(20): May or may not be N-glycosylated
  MISC_FEATURE (23)..(23): May or may not be N-glycosylated
  MISC_FEATURE (31)..(31): May or may not be N-glycosylated
  See specification as filed for detailed description of substitutions and preferred embodiments
ITCPPPMSVEHADIWVKSYSLYSRERYICNS

SEQ ID NO: 25 - protein, 4 aa, Artificial Sequence
  Description of Artificial Sequence Synthetic peptide
  IL-15Ra heterologous protease cleavage site recognized by furin protease.
  MOD_RES (2)..(3): Any amino acid
RXXR

SEQ ID NO: 26 - protein, 6 aa, Artificial Sequence
  Description of Artificial Sequence Synthetic peptide
  IL-15Ra heterologous protease cleavage site.
  MOD_RES (1)..(2): Any hydrophobic amino acid
  MOD_RES (5)..(6): Any non-acidic amino acid
  See specification as filed for detailed description of substitutions and preferred embodiments
XXPRXX

SEQ ID NO: 27 - protein, 436 aa, Artificial Sequence
  Description of Artificial Sequence Synthetic polypeptide
  synthetic sIL-15Ralpha-Fc fusion protein huIL15sRa205-Fc
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 431 aa, Artificial Sequence
  Description of Artificial Sequence Synthetic polypeptide
  synthetic sIL-15Ralpha-Fc fusion protein huIL15sRa200-Fc
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 5 aa, Artificial Sequence
  Description of Artificial Sequence Synthetic peptide
  BAP049-Clone-B HCDR1 (Kabat)
TYWMH

SEQ ID NO: 30 - protein, 17 aa, Artificial Sequence
  Description of Artificial Sequence Synthetic peptide
  BAP049-Clone-B HCDR2 (Kabat)
NIYPGTGGSNFDEKFKN

SEQ ID NO: 31 - protein, 8 aa, Artificial Sequence
  Description of Artificial Sequence Synthetic peptide
  BAP049-Clone-B HCDR3 (Kabat)
WTTGTGAY

SEQ ID NO: 32 - protein, 7 aa, Artificial Sequence
  Description of Artificial Sequence Synthetic peptide
  BAP049-Clone-B HCDR1 (Chothia)
GYTFTTY

SEQ ID NO: 33 - protein, 6 aa, Artificial Sequence
  Description of Artificial Sequence Synthetic peptide
  BAP049-Clone-B HCDR2 (Chothia)
YPGTGG

SEQ ID NO: 34 - protein, 8 aa, Artificial Sequence
  Description of Artificial Sequence Synthetic peptide
  BAP049-Clone-B HCDR3 (Chothia)
WTTGTGAY

SEQ ID NO: 35 - protein, 117 aa, Artificial Sequence
  Description of Artificial Sequence Synthetic polypeptide
  BAP049-Clone-B VH
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSS

SEQ ID NO: 36 - DNA, 351 bp, Artificial Sequence
  Description of Artificial Sequence Synthetic polynucleotide
  BAP049-Clone-B VH DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagc

SEQ ID NO: 37 - protein, 442 aa, Artificial Sequence
  Description of Artificial Sequence Synthetic polypeptide
  BAP049-Clone-B HC
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPDIAVEWESNGQPENNYKTTPPVLDSDGSFFLYSRLTVDKSRWQEGNVFSCSVMHEALHNHYTQKSLSLSLG

SEQ ID NO: 38 - DNA, 1329 bp, Artificial Sequence
  Description of Artificial Sequence Synthetic polynucleotide
  BAP049-Clone-B HC DNA
gaggtgcagctggtgcagtcaggcgccgaagtgaagaagcccggcgagtcactgagaattagctgtaaaggttcaggctacaccttcactacctactggatgcactgggtccgccaggctaccggtcaaggcctcgagtggatgggtaatatctaccccggcaccggcggctctaacttcgacgagaagtttaagaatagagtgactatcaccgccgataagtctactagcaccgcctatatggaactgtctagcctgagatcagaggacaccgccgtctactactgcactaggtggactaccggcacaggcgcctactggggtcaaggcactaccgtgaccgtgtctagcgctagcactaagggcccgtccgtgttccccctggcaccttgtagccggagcactagcgaatccaccgctgccctcggctgcctggtcaaggattacttcccggagcccgtgaccgtgtcctggaacagcggagccctgacctccggagtgcacaccttccccgctgtgctgcagagctccgggctgtactcgctgtcgtcggtggtcacggtgccttcatctagcctgggtaccaagacctacacttgcaacgtggaccacaagccttccaacactaaggtggacaagcgcgtcgaatcgaagtacggcccaccgtgcccgccttgtcccgcgccggagttcctcggcggtccctcggtctttctgttcccaccgaagcccaaggacactttgatgatttcccgcacccctgaagtgacatgcgtggtcgtggacgtgtcacaggaagatccggaggtgcagttcaattggtacgtggatggcgtcgaggtgcacaacgccaaaaccaagccgagggaggagcagttcaactccacttaccgcgtcgtgtccgtgctgacggtgctgcatcaggactggctgaacgggaaggagtacaagtgcaaagtgtccaacaagggacttcctagctcaatcgaaaagaccatctcgaaagccaagggacagccccgggaaccccaagtgtataccctgccaccgagccaggaagaaatgactaagaaccaagtctcattgacttgccttgtgaagggcttctacccatcggatatcgccgtggaatgggagtccaacggccagccggaaaacaactacaagaccacccctccggtgctggactcagacggatccttcttcctctactcgcggctgaccgtggataagagcagatggcaggagggaaatgtgttcagctgttctgtgatgcatgaagccctgcacaaccactacactcagaagtccctgtccctctccctggga

SEQ ID NO: 39 - protein, 17 aa, Artificial Sequence
  Description of Artificial Sequence Synthetic peptide
  BAP049-Clone-B LCDR1 (Kabat)
KSSQSLLDSGNQKNFLT

SEQ ID NO: 40 - protein, 7 aa, Artificial Sequence
  Description of Artificial Sequence Synthetic peptide
  BAP049-Clone-B LCDR2 (Kabat)
WASTRES

SEQ ID NO: 41 - protein, 9 aa, Artificial Sequence
  Description of Artificial Sequence Synthetic peptide
  BAP049-Clone-B LCDR3 (Kabat)
QNDYSYPYT

SEQ ID NO: 42 - protein, 13 aa, Artificial Sequence
  Description of Artificial Sequence Synthetic peptide
  BAP049-Clone-B LCDR1 (Chothia)
SQSLLDSGNQKNF

SEQ ID NO: 43 - protein, 3 aa, Artificial Sequence
  Description of Artificial Sequence Synthetic peptide
  BAP049-Clone-B LCDR2 (Chothia)
WAS

SEQ ID NO: 44 - protein, 6 aa, Artificial Sequence
  Description of Artificial Sequence Synthetic peptide
  BAP049-Clone-B LCDR3 (Chothia)
DYSYPY

SEQ ID NO: 45 - protein, 113 aa, Artificial Sequence
  Description of Artificial Sequence Synthetic polypeptide
  BAP049-Clone-B VL
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIK

SEQ ID NO: 46 - DNA, 339 bp, Artificial Sequence
  Description of Artificial Sequence Synthetic polynucleotide
  BAP049-Clone-B VL DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaag

SEQ ID NO: 47 - protein, 220 aa, Artificial Sequence
  Description of Artificial Sequence Synthetic polypeptide
  BAP049-Clone-B LC
EIVLTQSPATLSLSPGERATLSCKSSQSLLDSGNQKNFLTWYQQKPGKAPKLLIYWASTRESGVPSRFSGSGSGTDFTFTISSLQPEDIATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - DNA, 660 bp, Artificial Sequence
  Description of Artificial Sequence Synthetic polynucleotide
  BAP049-Clone-B LC DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtaaagcccctaagctgctgatctactgggcctctactagagaatcaggcgtgccctctaggtttagcggtagcggtagtggcaccgacttcaccttcactatctctagcctgcagcccgaggatatcgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 49 - protein, 5 aa, Artificial Sequence
  Description of Artificial Sequence Synthetic peptide
  BAP049-Clone-E HCDR1 (Kabat)
TYWMH

SEQ ID NO: 50 - protein, 17 aa, Artificial Sequence
  Description of Artificial Sequence Synthetic peptide
  BAP049-Clone-E HCDR2 (Kabat)
NIYPGTGGSNFDEKFKN

SEQ ID NO: 51 - protein, 8 aa, Artificial Sequence
  Description of Artificial Sequence Synthetic peptide
  BAP049-Clone-E HCDR3 (Kabat)
WTTGTGAY

SEQ ID NO: 52 - protein, 7 aa, Artificial Sequence
  Description of Artificial Sequence Synthetic peptide
  BAP049-Clone-E HCDR1 (Chothia)
GYTFTTY

SEQ ID NO: 53 - protein, 6 aa, Artificial Sequence
  Description of Artificial Sequence Synthetic peptide
  BAP049-Clone-E HCDR2 (Chothia)
YPGTGG

SEQ ID NO: 54 - protein, 8 aa, Artificial Sequence
  Description of Artificial Sequence Synthetic peptide
  BAP049-Clone-E HCDR3 (Chothia)
WTTGTGAY

SEQ ID NO: 55 - protein, 117 aa, Artificial Sequence; identical to SEQ ID NO: 35
  Description of Artificial Sequence Synthetic polypeptide
  BAP049-Clone-E  VH

SEQ ID NO: 56 - DNA, 351 bp, Artificial Sequence; identical to SEQ ID NO: 36
  Description of Artificial Sequence Synthetic polynucleotide
  BAP049-Clone-E VH DNA

SEQ ID NO: 57 - protein, 443 aa, Artificial Sequence
  Description of Artificial Sequence Synthetic polypeptide
  BAP049-Clone-E HC
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - DNA, 1329 bp, Artificial Sequence; identical to SEQ ID NO: 38
  Description of Artificial Sequence Synthetic polynucleotide
  BAP049-Clone-E HC DNA

SEQ ID NO: 59 - protein, 17 aa, Artificial Sequence
  Description of Artificial Sequence Synthetic peptide
  BAP049-Clone-E LCDR1 (Kabat)
KSSQSLLDSGNQKNFLT

SEQ ID NO: 60 - protein, 7 aa, Artificial Sequence
  Description of Artificial Sequence Synthetic peptide
  BAP049-Clone-E LCDR2 (Kabat)
WASTRES

SEQ ID NO: 61 - protein, 9 aa, Artificial Sequence
  Description of Artificial Sequence Synthetic peptide
  BAP049-Clone-E LCDR3 (Kabat)
QNDYSYPYT

SEQ ID NO: 62 - protein, 13 aa, Artificial Sequence
  Description of Artificial Sequence Synthetic peptide
  BAP049-Clone-E LCDR1 (Chothia)
SQSLLDSGNQKNF

SEQ ID NO: 63 - protein, 3 aa, Artificial Sequence
  Description of Artificial Sequence Synthetic peptide
  BAP049-Clone-E LCDR2 (Chothia)
WAS

SEQ ID NO: 64 - protein, 6 aa, Artificial Sequence
  Description of Artificial Sequence Synthetic peptide
  BAP049-Clone-E LCDR3 (Chothia)
DYSYPY

SEQ ID NO: 65 - protein, 113 aa, Artificial Sequence
  Description of Artificial Sequence Synthetic polypeptide
  BAP049-Clone-E VL
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIK

SEQ ID NO: 66 - DNA, 339 bp, Artificial Sequence
  Description of Artificial Sequence Synthetic polynucleotide
  BAP049-Clone-E VL DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaag

SEQ ID NO: 67 - protein, 220 aa, Artificial Sequence
  Description of Artificial Sequence Synthetic polypeptide
  BAP049-Clone-E LC
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - DNA, 660 bp, Artificial Sequence
  Description of Artificial Sequence Synthetic polynucleotide
  BAP049-Clone-E LC DNA
gagatcgtcctgactcagtcacccgctaccctgagcctgagccctggcgagcgggctacactgagctgtaaatctagtcagtcactgctggatagcggtaatcagaagaacttcctgacctggtatcagcagaagcccggtcaagcccctagactgctgatctactgggcctctactagagaatcaggcgtgccctctaggtttagcggtagcggtagtggcaccgacttcaccttcactatctctagcctggaagccgaggacgccgctacctactactgtcagaacgactatagctacccctacaccttcggtcaaggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 69 - DNA, 15 bp, Artificial Sequence
  Description of Artificial Sequence Synthetic oligonucleotide
  BAP049-Clone-B HCDR1 (Kabat)
acctactggatgcac

SEQ ID NO: 70 - DNA, 51 bp, Artificial Sequence
  Description of Artificial Sequence Synthetic oligonucleotide
  BAP049-Clone-B HCDR2 (Kabat)
aatatctaccccggcaccggcggctctaacttcgacgagaagtttaagaat

SEQ ID NO: 71 - DNA, 24 bp, Artificial Sequence
  Description of Artificial Sequence Synthetic oligonucleotide
  BAP049-Clone-B HCDR3 (Kabat)
tggactaccggcacaggcgcctac

SEQ ID NO: 72 - DNA, 21 bp, Artificial Sequence
  Description of Artificial Sequence Synthetic oligonucleotide
  BAP049-Clone-B  HCDR1 (Chothia)
ggctacaccttcactacctac

SEQ ID NO: 73 - DNA, 18 bp, Artificial Sequence
  Description of Artificial Sequence Synthetic oligonucleotide
  BAP049-Clone-B HCDR2 (Chothia)
taccccggcaccggcggc

SEQ ID NO: 74 - DNA, 24 bp, Artificial Sequence
  Description of Artificial Sequence Synthetic oligonucleotide
  BAP049-Clone-B HCDR3 (Chothia)
tggactaccggcacaggcgcctac

SEQ ID NO: 75 - DNA, 51 bp, Artificial Sequence
  Description of Artificial Sequence Synthetic oligonucleotide
  BAP049-Clone-B LCDR1 (Kabat)
aaatctagtcagtcactgctggatagcggtaatcagaagaacttcctgacc

SEQ ID NO: 76 - DNA, 21 bp, Artificial Sequence
  Description of Artificial Sequence Synthetic oligonucleotide
  BAP049-Clone-B LCDR2 (Kabat)
tgggcctctactagagaatca

SEQ ID NO: 77 - DNA, 27 bp, Artificial Sequence
  Description of Artificial Sequence Synthetic oligonucleotide
  BAP049-Clone-B LCDR3 (Kabat)
cagaacgactatagctacccctacacc

SEQ ID NO: 78 - DNA, 39 bp, Artificial Sequence
  Description of Artificial Sequence Synthetic oligonucleotide
  BAP049-Clone-B LCDR1 (Chothia)
agtcagtcactgctggatagcggtaatcagaagaacttc

SEQ ID NO: 79 - DNA, 9 bp, Artificial Sequence
  Description of Artificial Sequence Synthetic oligonucleotide
  BAP049-Clone-B LCDR2 (Chothia)
tgggcctct

SEQ ID NO: 80 - DNA, 18 bp, Artificial Sequence
  Description of Artificial Sequence Synthetic oligonucleotide
  BAP049-Clone-B LCDR3(Chothia)
gactatagctacccctac

SEQ ID NO: 81 - DNA, 15 bp, Artificial Sequence
  Description of Artificial Sequence Synthetic oligonucleotide
  BAP049-Clone-E HCDR1 (Kabat)
acctactggatgcac

SEQ ID NO: 82 - DNA, 51 bp, Artificial Sequence; identical to SEQ ID NO: 70
  Description of Artificial Sequence Synthetic oligonucleotide
  BAP049-Clone-E HCDR2 (Kabat)

SEQ ID NO: 83 - DNA, 24 bp, Artificial Sequence
  Description of Artificial Sequence Synthetic oligonucleotide
  BAP049-Clone-E HCDR3 (Kabat)
tggactaccggcacaggcgcctac

SEQ ID NO: 84 - DNA, 21 bp, Artificial Sequence
  Description of Artificial Sequence Synthetic oligonucleotide
  BAP049-Clone-E HCDR1 (Chothia)
ggctacaccttcactacctac

SEQ ID NO: 85 - DNA, 18 bp, Artificial Sequence
  Description of Artificial Sequence Synthetic oligonucleotide
  BAP049-Clone-E HCDR2 (Chothia)
taccccggcaccggcggc

SEQ ID NO: 86 - DNA, 24 bp, Artificial Sequence
  Description of Artificial Sequence Synthetic oligonucleotide
  BAP049-Clone-E HCDR3 (Chothia)
tggactaccggcacaggcgcctac

SEQ ID NO: 87 - DNA, 51 bp, Artificial Sequence; identical to SEQ ID NO: 75
  Description of Artificial Sequence Synthetic oligonucleotide
  BAP049-Clone-E LCDR1 (Kabat)

SEQ ID NO: 88 - DNA, 21 bp, Artificial Sequence
  Description of Artificial Sequence Synthetic oligonucleotide
  BAP049-Clone-E LCDR2 (Kabat)
tgggcctctactagagaatca

SEQ ID NO: 89 - DNA, 27 bp, Artificial Sequence
  Description of Artificial Sequence Synthetic oligonucleotide
  BAP049-Clone-E LCDR3 (Kabat)
cagaacgactatagctacccctacacc

SEQ ID NO: 90 - DNA, 39 bp, Artificial Sequence; identical to SEQ ID NO: 78
  Description of Artificial Sequence Synthetic oligonucleotide
  BAP049-Clone-E LCDR1 (Chothia)

SEQ ID NO: 91 - DNA, 9 bp, Artificial Sequence
  Description of Artificial Sequence Synthetic oligonucleotide
  BAP049-Clone-E LCDR2 (Chothia)
tgggcctct

SEQ ID NO: 92 - DNA, 18 bp, Artificial Sequence
  Description of Artificial Sequence Synthetic oligonucleotide
  BAP049-Clone-E LCDR3 (Chothia)
gactatagctacccctac

SEQ ID NO: 93 - protein, 10 aa, Artificial Sequence
  Description of Artificial Sequence Synthetic peptide
  BAP049-Clone-B/E HC Combined HCDR1 Kabat/Chothia
GYTFTTYWMH

SEQ ID NO: 94 - protein, 19 aa, Artificial Sequence
  Description of Artificial Sequence Synthetic peptide
  BAP049-Clone-B HC leader sequence
MAWVWTLPFLMAAAQSVQA

SEQ ID NO: 95 - protein, 20 aa, Artificial Sequence
  Description of Artificial Sequence Synthetic peptide
  BAP049-Clone-B LC Leader sequence
MSVLTQVLALLLLWLTGTRC

SEQ ID NO: 96 - protein, 19 aa, Artificial Sequence
  Description of Artificial Sequence Synthetic peptide
  BAP049-Clone-E HC leader sequence
MAWVWTLPFLMAAAQSVQA

SEQ ID NO: 97 - protein, 20 aa, Artificial Sequence
  Description of Artificial Sequence Synthetic peptide
  BAP049-Clone-E LC leader sequence
MSVLTQVLALLLLWLTGTRC

SEQ ID NO: 98 - protein, 327 aa, Artificial Sequence
  Description of Artificial Sequence Synthetic polypeptide
  IgG4 (S228P) mutant constant region amino acid sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 326 aa, Artificial Sequence
  Description of Artificial Sequence Synthetic polypeptide
  IgG4 (S228P) mutant constant region amino acid sequence lacing C-terminal lysine (K)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 330 aa, Artificial Sequence
  Description of Artificial Sequence Synthetic polypeptide
  IgG1 (N297A) mutant constant region amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 330 aa, Artificial Sequence
  Description of Artificial Sequence Synthetic polypeptide
  IgG1 (D265A, P329A) mutant constant region amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 330 aa, Artificial Sequence
  Description of Artificial Sequence Synthetic polypeptide
  IgG1 (L234A, L235A) mutant constant region amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 107 aa, Homo sapiens [sequence omitted]

SEQ ID NO: 105 - protein, 5 aa, Unknown
  Description of Unknown CTLA-4 binding motif
MYPPY

(19) United States
(12) Patent Application Publication
(10) Pub. No.: US 2022/0356222 A9
(43) Pub. Date: Nov. 10, 2022

(54) COMPLEXES OF IL-15 AND IL-15RALPHA IN COMBINATION WITH ANTI-PD-1 ANTIBODY MOLECULES AND USES THEREOF

(71) Applicant: NOVARTIS AG, Basel (CH)

(72) Inventor: Nancy LEWIS, Randolph, NJ (US)

(21) Appl. No.: 16/479,089

(22) PCT Filed: Jan. 19, 2018
(86) PCT No.: PCT/IB2018/050348

§ 371 Date: Jul. 18, 2019

Related U.S. Application Data
(60) Provisional application No. 62/448,460, filed on Jan. 20, 2017.

(51) Int. Cl. C07K 14/54; A61P 35/04; C07K 14/715; C07K 16/28; A61K 9/00

(52) U.S. Cl. CPC C07K 14/5443; A61P 35/04; C07K 14/7155; C07K 16/2818; A61K 9/0019; A61K 2039/545

(57) ABSTRACT

Dosing regimens for the administration of complexes comprising IL-15/IL-15Ra in combination with an anti-PD-1 antibody molecule to patients are disclosed. Such dosing regimens can be used for preventing, treating and/or managing disorders such as cancer.

### SEQUENCE LISTING

The instant application contains a Sequence Listing which has been filed electronically in ASCII format and is hereby incorporated by reference in its entirety. Said ASCII copy, created on Feb. 12, 2020, is named PAT057506-US-PCT SL.txt and is 98,963 bytes in size.

### FIELD

In one aspect, described herein are administration regimens for the administration of complexes comprising interleukin-15 (“IL-15”) covalently or noncovalently bound to IL-15 receptor alpha (“IL-15Ra”) to patients in order to enhance IL-15-mediated immune function in combination with an additional agent such as an anti-PD-1 antibody. In a specific aspect, the administration regimens of the combination are useful in the prevention, treatment, and/or management of disorders in which activating the immune system is beneficial, such as cancer.

### BACKGROUND

The cytokine, interleukin-15 (IL-15), is a member of the four alpha-helix bundle family of lymphokines produced by many cells in the body. IL-15 plays a pivotal role in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity.

The IL-15 receptor consists of three polypeptides, the type-specific IL-15 receptor alpha (“IL-15Ra”), the IL-2/IL-15 receptor beta (or CD122) (“β”), and the common gamma chain (or CD132) (“γ”) that is shared by multiple cytokine receptors. IL-15Ra is thought to be expressed by a wide variety of cell types, but not necessarily in conjunction with β and γ. IL-15 signaling has been shown to occur through the heterodimeric complex of IL-15Ra, β, and γ; through the heterodimeric complex of β and γ, or through a subunit, IL-15RX, found on mast cells.

IL-15 is a soluble protein, but endogenous IL-15 is not readily detectable in serum or body fluids as it occurs predominantly as a membrane-bound form that is expressed or acquired by several types of accessory cells. For instance, although IL-15 mRNA is detected in cells of both hematopoietic and non-hematopoietic lineage, T cells do not produce IL-15. Instead, IL-15 binds to the IL-15Ra, forming cell-surface complexes on T cells. IL-15 specifically binds to the IL-15Ra with high affinity via the “sushi domain” in exon 2 of the extracellular domain of the receptor. After trans-endosomal recycling and migration back to the cell surface, these IL-15 complexes acquire the property to activate bystander cells expressing the IL-15R βγ low-affinity receptor complex, inducing IL-15-mediated signaling via the Jak/Stat pathway. A wild-type soluble form of IL-15Ra (“sIL-15Ra”), which is cleaved at a cleavage site in the extracellular domain immediately distal to the transmembrane domain of the receptor has been observed. Tumor necrosis factor-alpha-converting enzyme (TACE/ADAM17) has been implicated as a protease involved in this process.

Based on its multifaceted role in the immune system, various therapies designed to modulate IL-15-mediated function have been explored. Recent reports suggest that IL-15, when complexed with the sIL-15Ra, or the sushi domain, maintains its immune enhancing function. Recombinant IL-15 and IL-15/IL-15Ra complexes have been shown to promote to different degrees the expansion of memory CD8 T cells and NK cells and enhance tumor rejection in various preclinical models. Furthermore, tumor targeting of IL-15 or IL-15/IL-15Ra complex containing constructs in mouse models, resulted in improved anti-tumor responses in either immunocompetent animals transplanted with syngeneic tumors or in T- and B cell-deficient SCID mice (retaining NK cells) injected with human tumor cell lines. Enhanced anti-tumor activity is thought to be dependent on increased half-life of the IL-15-containing moiety as well as the trans-presentation of IL-15 on the surface of tumor cells leading to enhanced NK and/or CD8 cytotoxic T cell expansion within the tumor. As such, tumor cells engineered to express IL-15 were also reported to promote rejection of established tumors by enhancing T cell and NK cell recruitment, proliferation and function (Zhang et al, (2009) PNAS USA. 106:7513-7518; Munger et al, (1995) Cell Immunol. 165(2):289-293; Evans et al, (1997) Cell Immunol. 179(1):66-73; Klebanoff et al, (2004) PNAS USA. 101(7):1969-74; Sneller et al, (2011) Blood.118(26):6845-6848; Zhang et al, (2012) J. Immunol. 188(12):6156-6164).

While IL-15 shows anti-tumor activity and increased survival in preclinical animal models, data also indicated it has the propensity to upregulate PD-1 on CD8+ T cells and increase IL-10, potentially limiting its therapeutic benefit. In a preclinical study, combination strategies with recombinant IL-15 and checkpoint modulators targeting the PD-1/PD-L1 axis reduced both PD-1 and IL-10 and increased overall survival compared to the monotherapy (Yu et al, (2010) Clin. Cancer Res. 2010:6019-28).

The ability of T cells to mediate an immune response against an antigen requires two distinct signaling interactions (Viglietta et al. (2007) Neurotherapeutics 4:666-675; Korman et al. (2007) Adv. Immunol. 90:297-339). First, an antigen that has been arrayed on the surface of antigen-presenting cells (APC) is presented to an antigen-specific naive CD4+T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The immune system is tightly controlled by a network of costimulatory and co-inhibitory ligands and receptors. These molecules provide the second signal for T cell activation and provide a balanced network of positive and negative signals to maximize immune responses against infection, while limiting immunity to self (Wang et al. (Epub Mar. 7, 2011) J. Exp. Med. 208(3):577-92; Lepenies et al. (2008) Endocrine, Metabolic & Immune Disorders--Drug Targets 8:279-288). Examples of costimulatory signals include the binding between the B7.1 (CD80) and B7.2 (CD86) ligands of the APC and the CD28 and CTLA-4 receptors of the CD4+ T-lymphocyte (Sharpe et al. (2002) Nature Rev. Immunol. 2:116-126; Lindley et al. (2009) Immunol. Rev. 229:307-321). Binding of B7.1 or B7.2 to CD28 stimulates T cell activation, whereas binding of B7.1 or B7.2 to CTLA-4 inhibits such activation (Dong et al. (2003) Immunolog. Res. 28(1):39-48; Greenwald et al. (2005) Ann. Rev. Immunol. 23:515-548). CD28 is constitutively expressed on the surface of T cells (Gross et al. (1992) J. Immunol. 149:380-388), whereas CTLA-4 expression is rapidly up-regulated following T-cell activation (Linsley et al. (1996) Immunity 4:535-543).

Other ligands of the CD28 receptor include a group of related B7 molecules, also known as the “B7 Superfamily” (Coyle et al. (2001) Nature Immunol. 2(3):203-209; Sharpe et al. (2002) Nature Rev. Immunol. 2:116-126; Collins et al. (2005) Genome Biol. 6:223.1-223.7; Korman et al. (2007) supra). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins et al. (2005) supra).

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. PD-1 is suggested to exist as a monomer, lacking the unpaired cysteine residue characteristic of other CD28 family members. PD-1 is expressed on activated B cells, T cells, and monocytes.

The PD-1 gene encodes a 55 kDa type I transmembrane protein (Agata et al. (1996) Int Immunol. 8:765-72). Although structurally similar to CTLA-4, PD-1 lacks the MYPPY motif (SEQ ID NO: [[236]] 105) that is important for B7-1 and B7-2 binding. Two ligands for PD-1 have been identified, PD-L1 (B7-H1) and PD-L2 (B7-DC), that have been shown to downregulate T cell activation upon binding to PD-1 (Freeman et al. (2000) J. Exp. Med. 192:1027-34; Carter et al. (2002) Eur. J. Immunol. 32:634-43). Both PD-L1 and PD-L2 are B7 homologs that bind to PD-1, but do not bind to other CD28 family members. PD-L1 is abundant in a variety of human cancers (Dong et al. (2002) Nat. Med. 8:787-9).

PD-1 is known as an immunoinhibitory protein that negatively regulates TCR signals (Ishida, Y. et al. (1992) EMBO J. 11:3887-3895; Blank, C. et al. (Epub 2006 Dec. 29) Immunol. Immunother. 56(5):739-745). The interaction between PD-1 and PD-L1 can act as an immune checkpoint, which can lead to, e.g., a decrease in tumor infiltrating lymphocytes, a decrease in T-cell receptor mediated proliferation, and/or immune evasion by cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100) Immune suppression can be reversed by inhibiting the local interaction of PD-1 with PD-L1 or PD-L2; the effect is additive when the interaction of PD-1 with PD-L2 is blocked as well (Iwai et al. (2002) PNAS USA 99:12293-7; Brown et al. (2003) J. Immunol. 170:1257-66).

Antibody inhibitors of immunological checkpoints, including PD-1 and PD-L1, have demonstrated significant antitumor activity in patients with various solid tumors with less toxicity than broad immune activators, such as IL-2 and IFN-α. Two monoclonal antibodies targeting PD-1, pembrolizumab and nivolumab, have demonstrated significant single agent activity in melanoma, non-small cell lung carcinoma (NSCLC), triple negative breast cancer (TNBC) and other solid tumors (Topalian et al, (2012) N. Engl. J. Med. 366:2443-54; Hamid et al, (2013), N. Engl. J. Med. 369:134-44; Topalian et al, (2014) J. Clin. Oncol. 32:1020-31; Seiwert et al, (2014) J. Clin. Oncol (Meeting Abstracts) 32(15s):6011; Powles et al, (2014) Nature 515:558-62; Garon et al, (2015) N. Engl. J. Med 372:2018-28; Moreno & Ribas (2015) Br. J. Cancer 112:1421-7; Robert et al, (2015) N. Engl. J. Med. 372:320-30). In patients with previously treated, unresectable melanoma the response rates to pembrolizumab and nivolumab were 34% and 31%, respectively; and the progression free survivals were 50 weeks and 9.7 months, respectively (Ribas et al, (2014) J. Clin. Oncol. (Meeting Abstracts) 32(15s):LBA9000; Topalian et al, (2014) supra). In patients with advanced, previously untreated non-small cell lung cancer the response rates to pembrolizumab and nivolumab were 26% and 30%, (Rizvi et al, (2014) J. Clin. Oncol. (Meeting Abstracts) 32(15s):8007; Gettinger et al, (2014) J. Clin. Oncol. (Meeting Abstracts) 32(15s):8024). Despite significant activity in some patients, as can be seen from the response rates detailed above, the majority of patients treated with single agent anti-PD-1 immunotherapy do not benefit from treatment.

It is believed that therapeutic approaches that enhance anti-tumor immunity could work more effectively when the immune response is optimized by targeting multiple components at one or more stages of an immune response, e.g., an anti-tumor immune response. For example, approaches that enhance cellular and humoral immune responses (e.g., by stimulating, e.g., disinhibiting, phagocytes and/or tumor infiltrating lymphocytes (e.g., NK cells and T cells)), while blocking tumor immunosuppressive signaling (e.g., by increasing macrophage polarization, increasing Treg depletion and/or decreasing myeloid-derived suppressive cells (MDSCs)) can result in a more effective and/or prolonged therapeutic response. Therefore combination therapies for cancer immunotherapy are desirable.

### SUMMARY

Accordingly, disclosed herein are combination therapies that remove the immunosuppressive effect in the tumor microenvironment and as such the combinations disclosed herein can provide a superior beneficial effect, e.g., in the treatment of a disorder, such as an enhanced anti-cancer effect, reduced toxicity and/or reduced side effects, compared to monotherapy administration of the therapeutic agents of the combination. For example, one or more of the therapeutic agents in the combination can be administered at a lower dosage, or for a shorter period of administration or less frequently, than would be required to achieve the same therapeutic effect compared to the monotherapy administration. Thus, compositions and methods for treating cancer and other immune disorders using combination therapies are disclosed.

In one aspect, the combination therapy comprises an agent that modulates the activity of immunoinhibitory proteins such as PD-1, in combination with immune enhancing agents such as IL-15 complexed with sIL-15Ra, to enhance the immune system. Such combination therapies can be used, e.g., for cancer immunotherapy and treatment of other conditions, such as chronic infection.

In an embodiment, provided herein are methods of treating (e.g., inhibiting, reducing, ameliorating, or preventing) a disorder, e.g., a hyperproliferative condition or disorder (e.g., a cancer) in a subject. The method includes administering to the subject an agent for enhancing IL-15-mediated immune function, comprising administering to subjects agents that induce IL-15 signal transduction and enhance IL-15-mediated immune function in combination with an anti-PD-1 antibody molecule, e.g., an anti-PD-1 antibody molecule as described herein. More specifically, provided herein are methods of treating (e.g., inhibiting, reducing, ameliorating, or preventing) a disorder, e.g., a hyperproliferative condition or disorder (e.g., a cancer) in a subject by administering to the subject an IL-15/IL-15Ra complex in combination with an anti-PD-1 antibody molecule. Also provided is an IL-15/IL-15Ra complex in combination with an anti-PD-1 antibody molecule for use in the treatment of (e.g., inhibiting, reducing, ameliorating, or preventing) a disorder, e.g., a hyperproliferative condition or disorder (e.g., a cancer) in a subject. Further provided is an IL-15/IL-15Ra complex in combination with an anti-PD-1 antibody molecule for use in the preparation of a medicament for the treatment of (e.g., inhibiting, reducing, ameliorating, or preventing) a disorder, e.g., a hyperproliferative condition or disorder (e.g., a cancer) in a subject.

In an embodiment, provided herein is a method of treating a cancer in a subject, the method comprising administering to the subject: (a) at least one initial dose of an IL-15/IL-15Ra complex followed by escalating doses of the IL-15/IL-15Ra complex; in combination with (b) an anti-PD-1 antibody molecule. Also provided herein is an IL-15/IL-15Ra complex and an anti-PD-1 antibody molecule for use in treating a cancer in a subject, wherein (a) the IL-15/IL-15Ra complex is administered to the subject at an initial dose, followed by escalating doses of the IL-15/IL-15Ra complex; in combination with (b) the anti-PD-1 antibody molecule. Further provided is an IL-15/IL-15Ra complex and an anti-PD-1 antibody molecule for use in the preparation of a medicament for the treatment of a cancer, wherein (a) the IL-15/IL-15Ra complex is administered to the subject at an initial dose, followed by escalating doses of the IL-15/IL-15Ra complex; in combination with (b) the anti-PD-1 antibody molecule.

In an embodiment, provided herein is a composition comprising a complex of IL-15 with a soluble form of IL-15Ra. In one embodiment, the IL-15 of the composition is human IL-15. The complex may comprise IL-15 covalently or noncovalently bound to a soluble form of IL-15Ra. In a particular embodiment the human IL-15 is noncovalently bonded to a soluble form of IL-15Ra. In a particular embodiment, the human IL-15 of the composition comprises the amino acid sequence of SEQ ID NO:1 in Table 1 or amino acid residues 49 to 162 of SEQ ID NO:1 in Table 1 and the soluble form of IL-15Ra comprises the amino acid sequence of SEQ ID NO:10. In some embodiments, the composition is a pharmaceutical composition.

In an embodiment, provided herein are antibody molecules (e.g., humanized antibody molecules) that bind to Programmed Death 1 (PD-1) with high affinity and specificity. Nucleic acid molecules encoding the antibody molecules, pharmaceutical compositions and dose formulations comprising the antibody molecules are also provided.

In an embodiment, provided herein are methods of treating a hyperproliferative condition or disorder, e.g. cancer, by administering an IL-15/IL-15Ra complex to a subject, e.g. as part of a therapeutic protocol e.g. by (a) administering an initial dose of an IL-15/IL-15Ra complex to a subject in a first treatment cycle; and administering further doses of the IL-15/IL-15Ra complex to the subject in respective, successive treatment cycles or (b) administering an initial dose of an IL-15/IL-15Ra complex to a subject in a first treatment cycle; and administering successively higher doses of the IL-15/IL-15Ra complex to the subject in respective, successive treatment cycles. For example, in one embodiment, an IL-15/IL-15Ra complex is administered to a subject at an initial dose in a first treatment cycle and then repeated administrations of the same dose are made in the second and repeated treatment cycles. In some embodiments, the dose of the first cycle is 0.25 μg/kg, 0.5 μg/kg, 1 μg/kg, 2 μg/kg, 4 μg/kg or 8 μg/kg followed by successive cycles of administration of the same dose. In certain embodiments, the dose of the first cycle is 1 μg/kg, followed by a dose of 1 μg/kg in the second cycle and 1 μg/kg in the third cycle. For example, the subject can receive the same dose once a week for three weeks, followed by a one-week break during each treatment cycle (28 days). In certain embodiments, the dose may be administered subcutaneously (SC). For example, in an alternative embodiment, if an IL-15/IL-15Ra complex is administered to a subject at an initial dose in a first treatment cycle, then the dose administered to the subject at the second cycle of the dosing regimen is increased relative to the dose administered during the first cycle, the dose administered to the subject during the third cycle is increased relative to the dose administered during the second cycle, the dose administered to the subject during the fourth cycle is increased relative to the dose administered during the third cycle, the dose administered to the subject during the fifth cycle is increased relative the dose administered the fourth cycle, and so on. In some embodiments, the dose of the first cycle is 0.25 μg/kg followed by successive cycles at dose levels of 0.5 μg/kg, 1 μg/kg, 2 μg/kg, 4 μg/kg and 8 μg/kg respectively. In certain embodiments, the dose of the first cycle is 1 μg/kg, followed by a dose of 2 μg/kg in the second cycle, 4 μg/kg in the third cycle and 8 μg/kg in the fourth cycle. For example, the subject can receive the same dose three times a week for two weeks, followed by a two-week break during each treatment cycle (28 days). In certain embodiments, the dose may be administered subcutaneously (SC).

In an embodiment, provided herein are methods of treating a hyperproliferative condition or disorder, e.g. cancer, by administering an anti-PD-1 antibody molecule to a subject, e.g. as part of a therapeutic protocol e.g. at a dose of about 300 mg to 400 mg of an anti-PD-1 antibody molecule once every three weeks or once every four weeks. In one embodiment, the dose is about 300 mg of an anti-PD-1 antibody molecule once every three weeks. In another embodiment, the dose is about 300 mg of an anti-PD-1 antibody molecule once every four weeks. In certain embodiments, the dose is about 400 mg of an anti-PD-1 antibody molecule once every four weeks. In certain embodiments, the dose may be administered intravenously (IV) as an infusion.

In another aspect, the invention features a composition (e.g., one or more compositions or dosage forms), that includes an IL-15/IL-15Ra complex and an anti-PD-1 antibody molecule (e.g., an IL-15/IL-15Ra complex and/or anti-PD-1 antibody molecule as described herein). Formulations, e.g., dosage formulations, and kits, e.g., therapeutic kits, that include an IL-15/IL-15Ra complex and an anti-PD-1 antibody molecule, are also described herein. In certain embodiments, the composition or formulation comprises 400 mg of an anti-PD-1 antibody molecule (e.g., an anti-PD-1 antibody molecule as described herein). In some embodiments, the composition or formulation is administered or used once every three weeks. In certain embodiments, the composition or formulation is administered or used once every four weeks.

In one aspect, provided herein is a method for enhancing IL-15-mediated immune function, comprising subcutaneously administering to a subject an IL-15/IL-15Ra complex in a dose regimen, wherein a dosing cycle in the regimen comprises: (a) subcutaneously administering a dose of the IL-15/IL-15Ra complex to the subject at set time intervals over a first period of time; and (b) no administration of the IL-15/IL-15Ra complex for a second period of time. In certain embodiments, the dose cycle is repeated 2, 3, 4, 5, 6, or more times, each time with either the same or an increased dose of the IL-15/IL-15Ra complex. In some embodiments, the IL-15/IL-15Ra complex is administered at a frequency of every day, every other day, every 3, 4, 5, 6 or 7 days. In certain embodiments, the IL-15/IL-15Ra is administered three times per week (e.g. Monday, Wednesday, Friday). In other embodiments, the first and second periods of time are different. In specific embodiments, the first period for administration of the IL-15/IL-15Ra complex is 1 week to 4 weeks long, 2 to 4 weeks, 2 to 3 weeks, or 1 to 2 weeks. In other embodiments, the first period for administration of the IL-15/IL-15Ra complex is 1 week, 2 weeks, 3 weeks or 4 weeks long. In some embodiments, the second period of time is 1 week to 2 months, 1 to 8 weeks, 2 to 8 weeks, 1 to 6 weeks, 2 to 6 weeks, 1 to 5 weeks, 2 to 5 weeks, 1 to 4 weeks, 2 to 4 weeks, 2 to 3 weeks, 1 to 2 weeks, 3 weeks, 2 weeks or 1 week long. In a specific embodiment, the first period is 3 weeks long and the second period is 1 week long. In some embodiments, the dose administered during the first period of the first cycle is the same and remains unchanged for subsequent cycles. For example, the dose of the first cycle and for each subsequent cycle is 0.1 μg/kg to 0.5 μg/kg, 0.25 μg/kg to 1 μg/kg, 0.5 μg/kg to 2 μg/kg, 1μg/kg to 4μg/kg, or 2 μg/kg to 8 μg/kg, more specifically 0.25 μg/kg, 0.5 μg/kg, 1 μg/kg, 2μg/kg, 4 μg/kg or 8 μg/kg. In a specific embodiment, the dose is 1 μg/kg for the first period of the first cycle and is 1 μg/kg for each subsequent cycle. In a further embodiment, the dose of the first cycle is lower than the dose used in one or more subsequent cycles of the dose regimen. In one embodiment the dose of the subsequent cycle is double that of the previous cycle. In one embodiment, the dose of the first cycle and each subsequent cycle is 0.1 μg/kg to 0.5 μg/kg, 0.25 μg/kg to 1μg/kg, 1 μg/kg to 5 μg/kg, or 5 μg/kg to 10 μg/kg. In another embodiment, the first dose of the first cycle and each subsequent cycle is 0.1 μg/kg to 0.5 μg/kg, 0.25 μg/kg to 1 μg/kg, 0.5 μg/kg to 2 μg/kg, 1 μg/kg to 4 μg/kg, or 2 μg/kg to 8 μg/kg. In another embodiment, the dose of the first cycle is 0.25 μg/kg, 0.5 μg/kg, 1 μg/kg, 2 μg/kg, 4 μg/kg or 8 μg/kg. In a certain embodiment, the dose of the first cycle and each subsequent cycle is 1 μg/kg, and 2 μg/kg, 4 μg/kg, 8 μg/kg, respectively.

In an embodiment, provided herein is a method for enhancing anti-tumor immunity by administering an anti-PD-1 antibody molecule to a subject, e.g. as part of a therapeutic protocol e.g. at a dose of about 300 mg to 400 mg of an anti-PD-1 antibody molecule. In some embodiments the anti-PD-1 antibody molecule can be administered once every three weeks or once every four weeks. In certain embodiments, the dose is about 400 mg of an anti-PD-1 antibody molecule and is administered once every four weeks. In certain embodiments, the dose is about 300 mg of an anti-PD-1 antibody molecule and is administered once every three weeks.

In one aspect, provided herein is a method for treating a hyperproliferative condition or disorder by administering a combination of an IL-15/IL-15Ra complex and an anti-PD-1 antibody molecule to a subject, e.g. as part of a therapeutic protocol e.g. by (a) SC administering the IL-15/IL-15Ra complex to the subject at an initial dose and administering an anti-PD-1 antibody molecule by IV infusion; (b) repeating the administration of the IL-15/IL-15Ra complex to the subject at set time intervals over a first period of time; (c) no administration of the IL-15/IL-15Ra complex for a second period of time; and (d) repeating steps (a) to (c) at the same or at an escalating dose of the IL-15/IL-15Ra complex. In some embodiments, the first period for administration of the IL-15/IL-15Ra complex is 1 week, 2 weeks, 3 weeks or 4 weeks long. In some embodiments, the second period for administration of the IL-15/IL-15Ra complex is 1 week, 2 weeks, 3 weeks or 4 weeks long. In other embodiments, the period for administration of the anti-PD-1 antibody molecule is 1 week, 2 weeks, 3 weeks, 4 weeks, 5 weeks or 6 weeks long. In a specific embodiment, the first period for administration of the IL-15/IL-15Ra complex is 3 weeks long and the second period is 1 weeks long, and the period for administration of the anti-PD-1 antibody molecule is 4 weeks. In certain embodiments, the dose of the first cycle is the same as the dose used in one or more subsequent cycles of the dose regimen. In an alternative embodiment, the dose of the first cycle is lower than the dose used in one or more subsequent cycles of the dose regimen. In one embodiment the dose of the subsequent cycle is double that of the previous cycle. In one embodiment, the dose of the IL-15/IL-15Ra complex at the initial dose and each subsequent or escalating dose is 0.25 μg/kg to 1 μg/kg, 1μg/kg to 5μg/kg, or 5 μg/kg to 10 μg/kg. In another embodiment, the dose of IL-15/IL-15Ra complex at the initial dose and each subsequent or escalating dose is 0.5 μg/kg to 2 μg/kg, 1 μg/kg to 4 μg/kg, or 2 μg/kg to 8 μg/kg. In another embodiment, the dose of the IL-15/IL-15Ra complex at the initial dose and each subsequent or escalating dose is 1 μg/kg, 2 μg/kg, 4 μg/kg or 8μg/kg. In another embodiment the dose of the anti-PD-1 antibody molecule administered in combination IL-15/IL-15Ra complex is about 300 mg to 400 mg. In some embodiments the anti-PD-1 antibody molecule can be administered once every three weeks or once every four weeks. In certain embodiments, the dose of the anti-PD-1 antibody molecule administered in combination with the IL-15/IL-15Ra complex is about 400 mg and is administered once every four weeks.

In one aspect, provided herein is a method for treating a hyperproliferative condition or disorder, e.g. cancer, by administering a combination of an IL-15/IL-15Ra complex and an anti-PD-1 antibody molecule to a subject, e.g. as part of a therapeutic protocol. In one embodiment, the therapeutic protocol comprises: (a) administering subcutaneously to the subject a dose of 1 μg/kg of the IL-15/IL-15Ra complex weekly over a first period of 3 weeks; and administering by IV infusion a dose of 400 mg of an anti-PD-1 antibody molecule on the same day as the first administration of the IL-15/IL-15Ra complex; and (b) after a second period of 1 week in which no IL-15/IL-15Ra complex is administered to the subject, administering subcutaneously to the subject the same dose of the IL-15/IL-15Ra complex weekly over a period of 3 weeks; and administering by IV infusion a dose of 400 mg of an anti-PD-1 antibody molecule on the same day as the first administration of the subsequent dose of the IL-15/IL-15Ra complex. In some embodiments the dose of the IL-15/IL-15Ra complex is 2 μg/kg, 4 μg/k or 8 μg/kg. In an alternative embodiment, the therapeutic protocol comprises: (a) administering subcutaneously to the subject a dose of 1 μg/kg of the IL-15/IL-15Ra complex three times per week (e.g. Monday, Wednesday, Friday) over a first period of 2 weeks; and administering by IV infusion a dose of 400 mg of an anti-PD-1 antibody molecule on the same day as the first administration of the IL-15/IL-15Ra complex; and (b) after a second period of 2 weeks in which no IL-15/IL-15Ra complex is administered to the subject, administering subcutaneously to the subject a higher dose of the IL-15/IL-15Ra complex three times per week (e.g. Monday, Wednesday, Friday) over a period of 2 weeks; and administering by IV infusion a dose of 400 mg of an anti-PD-1 antibody molecule on the same day as the first administration of the higher dose of the IL-15/IL-15Ra complex. In some embodiments the dose of the IL-15/IL-15Ra complex is increased to 2 μg/kg, 4 μg/kg, 8 μg/kg, respectively for each treatment cycle.

The dose regimen may be conducted one time, two times, three times, four times, five times, six times, seven times, eight times, nine times, ten times, or more, or 2 to 5 times, 5 to 10 times, 10 to 15 times, 15 to 20 times, 20 to 25 times or more. In certain embodiments, the dose escalation regimen is repeated for at least 2 months, at least 3 months, at least 4 months, at least 5 months, at least 6 months, at least 7 months, at least 8 months, at least 9 months, at least 10 months, at least 11 months, at least 1 year or more. In certain embodiments, the dose escalation regimen is repeated for 3 to 6 months, 6 to 9 months, 6 to 12 months, 1 to 1.5 years, 1 to 2 years, 1.5 to 2 years, or more.

In certain embodiments, the plasma levels of IL-15 and/or lymphocyte counts are monitored. In some embodiments, the subject is monitored for side effects such as a decrease in blood pressure and/or an increase in body temperature and/or an increase in cytokines in plasma. In certain embodiments, the dose of the IL-15/IL-15Ra complex administered during the first cycle of the dosing regimen is sequentially escalated if the subject does not have any adverse effects. In some embodiments, the dose of the IL-15/IL-15Ra complex administered during the first cycle of the dosing regimen is escalated for subsequent cycles of the dosing regimen if the subject does not experience any dose limiting toxicity, for example, grade 3 or 4 adverse events such as lymphopenia, grade 3 granulocytopenia, grade 3 leukocytosis (WBC>100,000/mm³), or organ dysfunction.

Non-limiting examples of disorders in which it is beneficial to enhance IL-15-mediated immune function include cancer, lymphopenia, immunodeficiencies, infectious diseases, and wounds. In a specific embodiment, the disorder in which it is beneficial to enhance IL-15-mediated immune function is cancer, including metastatic cancer. In another specific embodiment, the cancer in which it is beneficial to enhance IL-15-mediated immune function comprises a solid tumor such as a sarcoma, carcinoma or lymphoma. More specific examples of solid tumors include breast cancer, prostate cancer, lung cancer, liver cancer, pancreatic cancer, and melanoma. In another embodiment, the cancer is historically sensitive to treatment with an anti-PD-1 antibody molecule, for example, melanoma, non-small cell lung cancer or bladder cancer. In an alternative embodiment, the cancer is historically resistant to treatment with an anti-PD-1 antibody molecule.

The IL-15/IL-15Ra complex administered to a subject in accordance with the methods described herein may comprise wild-type IL-15 or an IL-15 derivative covalently or noncovalently bound to wild-type IL-15Ra or an IL-15Ra derivative. In one embodiment, the IL-15/IL-15Ra complex comprises wild-type IL-15 and wild-type IL-15Ra. In another embodiment, the IL-15/IL-15Ra complex comprises an IL-15 derivative and wild-type IL-15Ra. In another embodiment, the IL-15/IL-15Ra complex is in the naturally occurring heterodimeric form. In another embodiment, the IL-15 is human IL-15 and IL-15Ra is human IL-15Ra. In a specific embodiment, the human IL-15 comprises the amino acid sequence of SEQ ID NO: 1 in Table 1 or amino acid residues 49 to 162 of SEQ ID NO:1 in Table 1 and the human IL-15Ra comprises the amino acid sequence of SEQ ID NO: 6 in Table 1 or a fragment thereof. In another embodiment the IL-15 comprises the amino acid sequence of SEQ ID NO:1 in Table 1 or amino acid residues 49 to 162 of SEQ ID NO:1 in Table 1 and the IL-15Ra comprises the amino acid sequence of SEQ ID NO: 7 or 10 in Table 1. In specific embodiments, the human IL-15 comprises amino acid residues 49 to 162 of the amino acid sequence of SEQ ID NO: 1 in Table 1 and human IL-15Ra comprises the amino acid sequence of SEQ ID NO: 10 in Table 1.

In other embodiments, the IL-15Ra is glycosylated such that glycosylation accounts for at least or more than 20%, 30%, 40% or 50% of the mass of the IL-15Ra. In another embodiment, the IL-15/IL-15Ra complex comprises wild-type IL-15 and an IL-15Ra derivative. In another embodiment, the IL-15/IL-15Ra complex comprises an IL-15 derivative and an IL-15Ra derivative. In one embodiment, the IL-15Ra derivative is a soluble form of the wild-type IL-15Ra. In another embodiment, the IL-15Ra derivative comprises a mutation that inhibits cleavage by an endogenous protease. In a specific embodiment, the extracellular domain cleavage site of IL-15Ra is replaced with a cleavage site that is specifically recognized by a heterologous protease. In one embodiment, the extracellular domain cleavage site of IL-15Ra is replaced with a heterologous extracellular domain cleavage site (e.g., heterologous transmembrane domain that is recognized and cleaved by another enzyme unrelated to the endogenous processing enzyme that cleaves the IL-15Ra).

In one embodiment, the PD-1 inhibitor is an anti-PD-1 antibody molecule as described in U.S. Patent application no: 2015/0213769 entitled “Antibody Molecules to PD-1 and Uses Thereof”. In one embodiment, the anti-PD-1 antibody molecule comprises at least one antigen-binding region, e.g., a variable region or an antigen-binding fragment thereof, from an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

The anti-PD-1 antibody molecules described herein are preferred for use in the methods described herein, although other anti-PD-1 antibodies can be used instead, or in combination with an anti-PD-1 antibody molecule as described herein.

Uses of the Combination Therapies

The combinations disclosed herein can result in one or more of: an increase in antigen presentation, an increase in effector cell function (e.g., one or more of T cell proliferation, IFN-α secretion or cytolytic function), inhibition of regulatory T cell function, an effect on the activity of multiple cell types, such as regulatory T cell, effector T cells and NK cells), an increase in tumor infiltrating lymphocytes, an increase in T-cell receptor mediated proliferation, and a decrease in immune evasion by cancerous cells. In one embodiment, the use of an IL-15/11-15Ra complex in the combinations stimulates the immune response. In one embodiment, the use of a PD-1 inhibitor in the combinations inhibits, reduces or neutralizes one or more activities of PD-1, resulting in blockade or reduction of an immune checkpoint. Thus, such combinations can be used to treat or prevent disorders where enhancing an immune response in a subject is desired, e.g. cancer.

Accordingly, in another aspect, a method of modulating an immune response in a subject is provided. The method comprises administering to the subject a combination disclosed herein (e.g., a combination comprising a therapeutically effective amount of an IL-15/IL-15Ra complex and an anti-PD-1 antibody molecule), alone or in combination with one or more agents or procedures, such that the immune response in the subject is modulated. The subject can be a mammal, e.g., a primate, preferably a higher primate, e.g., a human (e.g., a patient having, or at risk of having, a disorder described herein). In one embodiment, the subject is in need of enhancing an immune response. In one embodiment, the subject has, or is at risk of, having a disorder described herein, e.g., a cancer or an infectious disorder as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

In one aspect, a method of treating (e.g., one or more of reducing, inhibiting, or delaying progression) a cancer or a tumor in a subject is provided. The method comprises administering to the subject a combination disclosed herein (e.g., a combination comprising a therapeutically effective amount of an Il-15/IL-15Ra complex and an anti-PD-1 antibody molecule).

In certain embodiments, the cancer treated with the combination, includes but is not limited to, a solid tumor, a hematological cancer (e.g., leukemia, lymphoma, myeloma, e.g., multiple myeloma), and a metastatic lesion. In one embodiment, the cancer is a solid tumor. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

In one embodiment, the cancer is chosen from a lung cancer (e.g., a non-small cell lung cancer (NSCLC) (e.g., a NSCLC with squamous and/or non-squamous histology, or a NSCLC adenocarcinoma)), a melanoma (e.g., an advanced melanoma), a renal cancer (e.g., a renal cell carcinoma), a liver cancer, a myeloma (e.g., a multiple myeloma), a prostate cancer, a bladder cancer, a breast cancer (e.g., a breast cancer that does not express one, two or all of estrogen receptor, progesterone receptor, or Her2/neu, e.g., a triple negative breast cancer), a colorectal cancer, a pancreatic cancer, a head and neck cancer (e.g., head and neck squamous cell carcinoma (HNSCC), anal cancer, gastro-esophageal cancer, thyroid cancer, cervical cancer, a lymphoproliferative disease (e.g., a post-transplant lymphoproliferative disease) or a hematological cancer, T-cell lymphoma, B-cell lymphoma, a non-Hodgkin lymphoma, or a leukemia (e.g., a myeloid leukemia or a lymphoid leukemia).

In another embodiment, the cancer is chosen from a carcinoma (e.g., advanced or metastatic carcinoma), melanoma or a lung carcinoma, e.g., a non-small cell lung carcinoma.

In one embodiment, the cancer is a lung cancer, e.g., a non-small cell lung cancer or small cell lung cancer.

In one embodiment, the cancer is a melanoma, e.g., an advanced melanoma. In one embodiment, the cancer is an advanced or unresectable melanoma that does not respond to other therapies. In other embodiments, the cancer is a melanoma with a BRAF mutation (e.g., a BRAF V600 mutation). In yet other embodiments, the combination disclosed herein (e.g., the combination comprising the anti-PD-1 antibody molecule) is administered after treatment with an anti-CTLA4 antibody (e.g., ipilimumab) with or without a BRAF inhibitor (e.g., vemurafenib or dabrafenib).

In another embodiment, the cancer is a hepatocarcinoma, e.g., an advanced hepatocarcinoma, with or without a viral infection, e.g., a chronic viral hepatitis.

In another embodiment, the cancer is a prostate cancer, e.g., an advanced prostate cancer. In yet another embodiment, the cancer is a myeloma, e.g., multiple myeloma. In yet another embodiment, the cancer is a renal cancer, e.g., a renal cell carcinoma (RCC) (e.g., a metastatic RCC or clear cell renal cell carcinoma (CCRCC)).

In one embodiment, the cancer microenvironment has an elevated level of PD-L1 expression. Alternatively, or in combination, the cancer microenvironment can have increased IFNα and/or CD8 expression.

In a certain embodiment, the subject has a cancer that is historically sensitive to treatment with an anti-PD-1 antibody molecule. For example, NSCLC, melanoma or bladder cancer. In an alternative embodiment, the subject has a cancer that is historically resistant to treatment with an anti-PD-1 antibody molecule.

In some embodiments, the subject has, or is identified as having, a tumor that has one or more of high PD-L1 level or expression, or as being Tumor Infiltrating Lymphocyte (TIL)+ (e.g., as having an increased number of TILs), or both. In certain embodiments, the subject has, or is identified as having a tumor that has high PD-L1 level or expression and that is TIL+. In some embodiments, the methods described herein further include identifying a subject based on having a tumor that has one or more of high PD-L1 level or expression, or as being TIL+, or both. In certain embodiments, the methods described herein further include identifying a subject based on having a tumor that has high PD-L1 level or expression and as being TIL+. In some embodiments, tumors that are TIL+are positive for CD8 and IFNγ. In some embodiments, the subject has, or is identified as having, a high percentage of cells that are positive for one, two or more of PD-L1, CD8, and/or IFNγ. In certain embodiments, the subject has or is identified as having a high percentage of cells that are positive for all of PD-L1, CD8, and IFNγ.

In a further aspect, the invention provides a method of treating an infectious disease in a subject, comprising administering to a subject a combination as described herein, e.g., a combination comprising a therapeutically effective amount of an IL-15/IL-15Ra complex and an anti-PD-1 antibody molecule described herein. In one embodiment, the infection disease is chosen from hepatitis (e.g., hepatitis C infection), or sepsis.

The combinations as described herein can be administered to the subject systemically (e.g., orally, parenterally, subcutaneously, intravenously, rectally, intramuscularly, intraperitoneally, intranasally, transdermally, or by inhalation or intracavitary installation).

In certain embodiments, the methods and compositions described herein are administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation (e.g., gamma irradiation). In other embodiments, the additional therapy is surgery or radiation, or a combination thereof. In other embodiments, the additional therapy is a therapy targeting one or more of PI3K/AKT/mTOR pathway, an HSP90 inhibitor, or a tubulin inhibitor.

### BRIEF DESCRIPTION OF THE FIGURES

FIG. 1: This figure shows the additive or synergistic effect of administration of a combination of an IL-15/IL-15Ra complex with an anti-PD-1 antibody molecule on IL-2 production in a Staphylococcal enterotoxin B (SEB) assay. IL-2 expression was measured in 4 donor PBMC preparations with the IL-15/IL-15Ra complex either added on the same day as PDR001(combo same time') or with freshly prepared IL-15/IL-15Ra complex added to the culture 72 hr post anti-PD-1 antibody molecule administration (combo sequential'). The assay was performed in triplicate on 4 different donors (E-012, E421, E444 and 1011) and the results are shown in FIGS. 1A-B, 1C-D, 1E-F and 1G-H, respectively.

### DETAILED DESCRIPTION

Disclosed herein, at least in part, are complexes of IL-15 with IL-15Ra as well as antibody molecules (e g , humanized antibody molecules) that bind to Programmed Death 1 (PD-1) with high affinity and specificity. Nucleic acid molecules encoding the IL-15/IL-15Ra complexes and antibody molecules, expression vectors, host cells and methods for making the IL-15/IL-15Ra complexes and antibody molecules are also provided. Pharmaceutical compositions and dose formulations comprising the IL-15/IL-15Ra complexes and antibody molecules are also provided. The IL-15/IL-15Ra complexes and anti-PD-1 antibody molecules disclosed herein can be used alone or in combination to treat, prevent and/or diagnose disorders, such as cancerous disorders (e.g., solid and soft-tissue tumors), as well as infectious diseases (e.g., chronic infectious disorders or sepsis). Thus, a combination composition and methods for treating various disorders including cancer and/or infectious diseases, using a combination of IL-15/IL-15Ra complexes and anti-PD-1 antibody molecules are disclosed herein. In certain embodiments, the IL-15/IL-15Ra complex is administered in an escalating dose regimen. In certain embodiments, the anti-PD-1 antibody molecule is administered or used at a flat or fixed dose.

Without wishing to be bound by theory, it is believed that therapeutic approaches that enhance anti-tumor immunity work more effectively when the immune response is optimized via multiple targets at different stages of the immune response. For example, approaches that result in enhancement of IL-15 mediated immune function combined with approaches that regulate immune checkpoint pathways can result in a more effective and/or prolonged therapeutic response.

Terminology

Additional terms are defined below and throughout the application.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The terms “disease” and “disorder” are used interchangeably to refer to a condition, in particular, a pathological condition. In certain embodiments, the terms “disease” and “disorder” are used interchangeably to refer to a disease affected by IL-15 signal transduction and/or a disease affected by the promotion of an immune effector response.

As used herein, the terms “treat”, “treatment” and “treating” refer to the reduction or amelioration of the progression, severity and/or duration of a disorder, e.g., a proliferative disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of a proliferative disorder, such as growth of a tumor, not necessarily discernible by the patient. In other embodiments the terms “treat”, “treatment” and “treating” -refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

As used herein, the terms “therapies” and “therapy” can refer to any protocol(s), method(s), compositions, formulations, and/or agent(s) that can be used in the prevention, treatment, management, or amelioration of a disease, e.g., cancer, infectious disease, lymphopenia, and immunodeficiencies, or a symptom associated therewith. In certain embodiments, the terms “therapies” and “therapy” refer to biological therapy, supportive therapy, and/or other therapies useful in treatment, management, prevention, or amelioration of a disease or a symptom associated therewith known to one of skill in the art.

As used herein, the terms “specifically binds,” “specifically recognizes” and analogous terms in the context of a receptor (e.g., IL-15Ra or IL-15 receptor βγ) and a ligand (e.g., IL-15) interaction refer to the specific binding or association between the ligand and receptor. Preferably, the ligand has higher affinity for the receptor than for other molecules. In a specific embodiment, the ligand is wild-type IL-15 and the receptor is wild-type IL-15Ra. In another specific embodiment, the ligand is the wild-type IL-15/IL-15Ra complex and the receptor is the 13y receptor complex. In a further embodiment, the IL-15/IL-15Ra complex binds to the 13y receptor complex and activates IL-15 mediated signal transduction. Ligands that specifically bind a receptor can be identified, for example, by immunoassays, BIAcore^{TM}, or other techniques known to those of skill in the art.

As used herein, the term “immunospecifically binds” and “specifically binds” in the context of antibodies refer to molecules that specifically bind to an antigen (e.g., an epitope or an immune complex) and do not specifically bind to another molecule. A molecule that specifically binds to an antigen may bind to other antigens with a lower affinity as determined by, e.g., immunoassays, BIAcore™ or other assays known in the art. In a specific embodiment, molecules that bind to an antigen do not cross-react with other antigens.

By “a combination” or “in combination with,” it is not intended to imply that the therapy or the therapeutic agents must be administered at the same time and/or formulated for delivery together, although these methods of delivery are within the scope described herein. The therapeutic agents in the combination can be administered concurrently with, prior to, or subsequent to, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

The term “inhibition,” “inhibitor,” or “antagonist” includes a reduction in a certain parameter, e.g., an activity, of a given molecule, e.g., an immune checkpoint inhibitor. For example, inhibition of an activity, e.g., a PD-1 or PD-L1 activity, of at least 5%, 10%, 20%, 30%, 40% or more is included by this term. Thus, inhibition need not be 100%.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Immune effector” or “effector” “function” or “response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The compositions and methods of the present invention encompass polypeptides and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the wild-type sequence, or are encoded by a substantially identical nucleotide sequence, and are capable of having one or more activities of the wild-type sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 70%, preferably at least 80%, more preferably at least 90%, 95%, and even more preferably at least 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available from the NCBI), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score =100, word length =12 to obtain nucleotide sequences homologous to a nucleic acid (SEQ ID NO: 1) molecules of the invention. BLAST protein searches can be performed with the XBLAST program, score=50, word length=3 to obtain amino acid sequences homologous to protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used (available from the NBCI).

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: 1) low stringency hybridization conditions in 6X sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules of the present invention may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of wild-type amino acids. Exemplary amino acids include wild-type amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a nonnatural arrangement.

The term “isolated,” as used herein, refers to material that is removed from its original or native environment (e.g., the natural environment if it is wild-type). For example, a wild-type polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

Various aspects of the invention are described in further detail below. Additional definitions are set out throughout the specification.

IL-15

As used herein, the terms “IL-15” and “interleukin-15” refer to a wild-type IL-15 or an IL-15 derivative. As used herein, the terms “wild-type IL-15” and “wild-type interleukin-15” in the context of proteins or polypeptides refer to any mammalian interleukin-15 amino acid sequences, including immature or precursor and mature forms. Non-limiting examples of GeneBank Accession Nos. for the amino acid sequence of various species of wild-type mammalian interleukin-15 include NP_000576 (human, immature form), CAA62616 (human, immature form), NP_001009207 (Fells catus, immature form), AAB94536 (Rattus norvegicus, immature form), AAB41697 (Rattus norvegicus, immature form), NP_032383 (Mus musculus, immature form), AAR19080 (canine), AAB60398 (Macaca mulatta, immature form), AAI00964 (human, immature form), AAH23698 (Mus musculus, immature form), and AAH18149 (human). The amino acid sequence of the immature/precursor form of human IL-15, which comprises the long signal peptide (underlined) and the mature human IL-15 (italicized), as provided in SEQ ID NO: 1 in Table 1. In some embodiments, the IL-15 is the immature or precursor form of a mammalian IL-15. In other embodiments, IL-15 is the mature form of a mammalian IL-15. In a specific embodiment, the IL-15 is the precursor form of human IL-15. In another embodiment, the IL-15 is the mature form of human IL-15. In one embodiment, the IL-15 protein/polypeptide is isolated or purified.

As used herein, the terms “IL-15” and “interleukin-15” in the context of nucleic acids refer to any nucleic acid sequences encoding mammalian interleukin-15, including the immature or precursor and mature forms. Non-limiting examples of GeneBank Accession Nos. for the nucleotide sequence of various species of wild-type mammalian IL-15 include NM_000585 (human), NM_008357 (Mus musculus), and RNU69272 (Rattus norvegicus). The nucleotide sequence encoding the immature/precursor form of human IL-15, which comprises the nucleotide sequence encoding the long signal peptide (underlined) and the nucleotide sequence encoding the mature human IL-15 (italicized), as provided in SEQ ID NO: 2 in Table 1. In a specific embodiment, the nucleic acid is an isolated or purified nucleic acid. In some embodiments, nucleic acids encode the immature or precursor form of a mammalian IL-15. In other embodiments, nucleic acids encode the mature form of a mammalian IL-15. In a specific embodiment, nucleic acids encoding IL-15 encode the precursor form of human IL-15. In another embodiment, nucleic acids encoding IL-15 encode the mature form of human IL-15.

As used herein, the terms “IL-15 derivative” and “interleukin-15 derivative” in the context of proteins or polypeptides refer to: (a) a polypeptide that is at least 75%, 80%, 85%, 90%, 95%, 98% or 99% identical to a wild-type mammalian IL-15 polypeptide; (b) a polypeptide encoded by a nucleic acid sequence that is at least 75%, 80%, 85%, 90%, 95%, 98% or 99% identical a nucleic acid sequence encoding a wild-type mammalian IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a wild-type mammalian IL-15 polypeptide; (d) a polypeptide encoded by nucleic acids can hybridize under high or medium stringency hybridization conditions to nucleic acids encoding a wild-type mammalian IL-15 polypeptide; (e) a polypeptide encoded by a nucleic acid sequence that can hybridize under high or medium stringency hybridization conditions to a nucleic acid sequence encoding a fragment of a wild-type mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; and/or (f) a fragment of a wild-type mammalian IL-15 polypeptide. IL-15 derivatives also include a polypeptide that comprises the amino acid sequence of a mature form of a mammalian IL-15 polypeptide and a heterologous signal peptide amino acid sequence. In a specific embodiment, an IL-15 derivative is a derivative of a wild-type human IL-15 polypeptide. In another embodiment, an IL-15 derivative is a derivative of an immature or precursor form of human IL-15 polypeptide. In another embodiment, an IL-15 derivative is a derivative of a mature form of human IL-15 polypeptide. In another embodiment, an IL-15 derivative is the IL-15N72D described in, e.g., Zhu et al., (2009), J. Immunol. 183: 3598 or U.S. Pat. No. 8,163,879. In another embodiment, an IL-15 derivative is one of the IL-15 variants described in U.S. Pat. No. 8,163,879. In one embodiment, an IL-15 derivative is isolated or purified.

In a preferred embodiment, IL-15 derivatives retain at least 75%, 80%, 85%, 90%, 95%, 98% or 99% of the function of wild-type mammalian IL-15 polypeptide to bind IL-15Ra polypeptide, as measured by assays well known in the art, e.g., ELISA, BIAcore™, co-immunoprecipitation. In another preferred embodiment, IL-15 derivatives retain at least 75%, 80%, 85%, 90%, 95%, 98% or 99% of the function of wild-type mammalian IL-15 polypeptide to induce IL-15-mediated signal transduction, as measured by assays well-known in the art, e.g., electromobility shift assays, ELISAs and other immunoassays. In a specific embodiment, IL-15 derivatives bind to IL-15Ra and/or IL-15Rβγ as assessed by, e.g., ligand/receptor binding assays well-known in the art. Percent identity can be determined using any method known to one of skill in the art and as described supra.

As used herein, the terms “IL-15 derivative” and “interleukin-15 derivative” in the context of nucleic acids refer to: (a) a nucleic acid sequence that is at least 75%, 80%, 85%, 90%, 95%, 98% or 99% identical to the nucleic acid sequence encoding a mammalian IL-15 polypeptide; (b) a nucleic acid sequence encoding a polypeptide that is at least 75%, 80%, 85%, 90%, 95%, 98% or 99% identical the amino acid sequence of a wild-type mammalian IL-15 polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid base mutations (i.e., additions, deletions and/or substitutions) relative to the nucleic acid sequence encoding a mammalian IL-15 polypeptide; (d) a nucleic acid sequence that hybridizes under high or medium stringency hybridization conditions to a nucleic acid sequence encoding a mammalian IL-15 polypeptide; (e) a nucleic acid sequence that hybridizes under high or medium stringency hybridization conditions to a fragment of a nucleic acid sequence encoding a mammalian IL-15 polypeptide; and/or (f) a nucleic acid sequence encoding a fragment of a nucleic acid sequence encoding a mammalian IL-15 polypeptide. In a specific embodiment, an IL-15 derivative in the context of nucleic acids is a derivative of a nucleic acid sequence encoding a human IL-15 polypeptide. In another embodiment, an IL-15 derivative in the context of nucleic acids is a derivative of a nucleic acid sequence encoding an immature or precursor form of a human IL-15 polypeptide. In another embodiment, an IL-15 derivative in the context of nucleic acids is a derivative of a nucleic acid sequence encoding a mature form of a human IL-15 polypeptide. In another embodiment, an IL-15 derivative in the context of nucleic acids is the nucleic acid sequence encoding the IL-15N72D described in, e.g., Zhu et al., (2009; supra), or U.S. Pat. No. 8,163,879. In another embodiment, an IL-15 derivative in the context of nucleic acids is the nucleic acid sequence encoding one of the IL-15 variants described in U.S. Pat. No. 8,163,879.

IL-15 derivative nucleic acid sequences include codon-optimized nucleic acid sequences that encode mammalian IL-15 polypeptide, including mature and immature forms of IL-15 polypeptide. In other embodiments, IL-15 derivative nucleic acids include nucleic acids that encode mammalian IL-15 RNA transcripts containing mutations that eliminate potential splice sites and instability elements (e.g., A/T or A/U rich elements) without affecting the amino acid sequence to increase the stability of the mammalian IL-15 RNA transcripts. In one embodiment, the IL-15 derivative nucleic acid sequences include the codon-optimized nucleic acid sequences described in WO2007/084342. In certain embodiments, the IL-15 derivative nucleic acid sequence is the codon-optimized sequence in SEQ ID NO: 4 in Table 1 (the amino acid sequence encoded by such a nucleic acid sequence is provided in SEQ ID NO: 5 in Table 1).

In a preferred embodiment, IL-15 derivative nucleic acid sequences encode proteins or polypeptides that retain at least 75%, 80%, 85%, 90%, 95%, 98% or 99% of the function of a wild-type mammalian IL-15 polypeptide to bind IL-15Ra, as measured by assays well known in the art, e.g., ELISA, BIAcore™, co-immunoprecipitation. In another preferred embodiment, IL-15 derivative nucleic acid sequences encode proteins or polypeptides that retain at least 75%, 80%, 85%, 90%, 95%, 98% or 99% of the function of a wild-type mammalian IL-15 polypeptide to induce IL-15-mediated signal transduction, as measured by assays well-known in the art, e.g., electromobility shift assays, ELISAs and other immunoassays. In a specific embodiment, IL-15 derivative nucleic acid sequences encode proteins or polypeptides that bind to IL-15Ra and/or IL-15Rβγ as assessed by, e.g., ligand/receptor assays well-known in the art.

IL-15Ra

As used herein, the terms “IL-15Ra” and “interleukin-15 receptor alpha” refer to a wild-type IL-15Ra, an IL-15Ra derivative, or a wild-type IL-15Ra and an IL-15Ra derivative. As used herein, the terms “wild-type IL-15Ra” and “wild-type interleukin-15 receptor alpha” in the context of proteins or polypeptides refer to any mammalian interleukin-15 receptor alpha (“IL-15Ra”) amino acid sequence, including immature or precursor and mature forms and isoforms. Non-limiting examples of GeneBank Accession Nos. for the amino acid sequence of various wild-type mammalian IL-15Ra include NP_002180 (human), ABK41438 (Macaca mulatta), NP_032384 (Mus musculus), Q60819 (Mus musculus), CAI41082 (human). The amino acid sequence of the immature form of the full length human IL-15Ra, which comprises the signal peptide (underlined) and the mature human IL-15Ra (italicized), as provided in SEQ ID NO: 6 in Table 1. The amino acid sequence of the immature form of the soluble human IL-15Ra, which comprises the signal peptide (underlined) and the mature human soluble IL-15Ra (italicized), as provided in SEQ ID NO:7 in Table 1. In some embodiments, IL-15Ra is the immature form of a mammalian IL-15Ra polypeptide. In other embodiments, IL-15Ra is the mature form of a mammalian IL-15Ra polypeptide. In certain embodiments, IL-15Ra is the soluble form of mammalian IL-15Ra polypeptide. In other embodiments, IL-15Ra is the full-length form of a mammalian IL-15Ra polypeptide. In a specific embodiment, IL-15Ra is the immature form of a human IL-15Ra polypeptide. In another embodiment, IL-15Ra is the mature form of a human IL-15Ra polypeptide. In certain embodiments, IL-15Ra is the soluble form of human IL-15Ra polypeptide. In other embodiments, IL-15Ra is the full-length form of a human IL-15Ra polypeptide. In one embodiment, an IL-15Ra protein or polypeptide is isolated or purified.

As used herein, the terms “IL-15Ra” and “interleukin-15 receptor alpha” in the context of nucleic acids refer to any nucleic acid sequences encoding mammalian interleukin-15 receptor alpha, including the immature or precursor and mature forms. Non-limiting examples of GeneBank Accession Nos. for the nucleotide sequence of various species of wild-type mammalian IL-15Ra include NM_002189 (human), EF033114 (Macaca mulatta), and NM_008358 (Mus musculus). The nucleotide sequence encoding the immature form of wild-type human IL-15Ra, which comprises the nucleotide sequence encoding the signal peptide (underlined) and the nucleotide sequence encoding the mature human IL-15Ra (italicized), as provided in SEQ ID NO: 8 in Table 1. The nucleotide sequence encoding the immature form of soluble human IL-15Ra protein or polypeptide, which comprises the nucleotide sequence encoding the signal peptide (underlined) and the nucleotide sequence encoding the mature human soluble IL-15Ra (italicized), as provided in SEQ ID NO: 9 in Table 1). In a specific embodiment, the nucleic acid is an isolated or purified nucleic acid. In some embodiments, nucleic acids encode the immature form of a mammalian IL-15Ra polypeptide. In other embodiments, nucleic acids encode the mature form of a mammalian IL-15Ra polypeptide. In certain embodiments, nucleic acids encode the soluble form of a mammalian IL-15Ra polypeptide. In other embodiments, nucleic acids encode the full-length form of a mammalian IL-15Ra polypeptide. In a specific embodiment, nucleic acids encode the precursor form of human IL-15 polypeptide. In another embodiment, nucleic acids encode the mature of human IL-15 polypeptide. In certain embodiments, nucleic acids encode the soluble form of a human IL-15Ra polypeptide. In other embodiments, nucleic acids encode the full-length form of a human IL-15Ra polypeptide.

As used herein, the terms “IL-15Ra derivative” and “interleukin-15 receptor alpha derivative” in the context of a protein or polypeptide refer to: (a) a polypeptide that is at least 75%, 80%, 85%, 90%, 95%, 98% or 99% identical to a wild-type mammalian IL-15 polypeptide; (b) a polypeptide encoded by a nucleic acid sequence that is at least 75%, 80%, 85%, 90%, 95%, 98% or 99% identical a nucleic acid sequence encoding a wild-type mammalian IL-15Ra polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a wild-type mammalian IL-15Ra polypeptide; (d) a polypeptide encoded by a nucleic acid sequence that can hybridize under high or medium stringency hybridization conditions to a nucleic acid sequence encoding a wild-type mammalian IL-15Ra polypeptide; (e) a polypeptide encoded by a nucleic acid sequence that can hybridize under high or medium stringency hybridization conditions to nucleic acid sequences encoding a fragment of a wild-type mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; (f) a fragment of a wild-type mammalian IL-15Ra polypeptide; and/or (g) a specific IL-15Ra derivative described herein. IL-15Ra derivatives also include a polypeptide that comprises the amino acid sequence of a mature form of mammalian IL-15Ra polypeptide and a heterologous signal peptide amino acid sequence. In a specific embodiment, an IL-15Ra derivative is a derivative of a wild-type human IL-15Ra polypeptide. In another embodiment, an IL-15Ra derivative is a derivative of an immature form of human IL-15 polypeptide. In another embodiment, an IL-15Ra derivative is a derivative of a mature form of human IL-15 polypeptide. In one embodiment, an IL-15Ra derivative is a soluble form of a mammalian IL-15Ra polypeptide. In other words, in certain embodiments, an IL-15Ra derivative includes soluble forms of mammalian IL-15Ra, wherein those soluble forms are not naturally occurring. Other examples of IL-15Ra derivatives include the truncated, soluble forms of human IL-15Ra described herein. In a specific embodiment, an IL-15Ra derivative is purified or isolated.

In a preferred embodiment, IL-15Ra derivatives retain at least 75%, 80%, 85%, 90%, 95%, 98% or 99% of the function of a wild-type mammalian IL-15Ra polypeptide to bind an IL-15 polypeptide, as measured by assays well known in the art, e.g., ELISA, BIAcore^{TM}, co-immunoprecipitation. In another preferred embodiment, IL-15Ra derivatives retain at least 75%, 80%, 85%, 90%, 95%, 98% or 99% of the function of a wild-type mammalian IL-15Ra polypeptide to induce IL-15-mediated signal transduction, as measured by assays well-known in the art, e.g., electromobility shift assays, ELISAs and other immunoassays. In a specific embodiment, IL-15Ra derivatives bind to IL-15 as assessed by methods well-known in the art, such as, e.g., ELISAs.

As used herein, the terms “IL-15Ra derivative” and “interleukin-15 receptor alpha derivative” in the context of nucleic acids refer to: (a) a nucleic acid sequence that is at least 75%, 80%, 85%, 90%, 95%, 98% or 99% identical to the nucleic acid sequence encoding a mammalian IL-15Ra polypeptide; (b) a nucleic acid sequence encoding a polypeptide that is at least 75%, 80%, 85%, 90%, 95%, 98% or 99% identical the amino acid sequence of a wild-type mammalian IL-15Ra polypeptide; (c) a nucleic acid sequence that contains 1, 2, 3, 4, 5, 6, 7, 8, 9, 10, 11, 12, 13, 14, 15, 16, 17, 18, 19, 20 or more nucleic acid mutations (i.e., additions, deletions and/or substitutions) relative to the nucleic acid sequence encoding a mammalian IL-15Ra polypeptide; (d) a nucleic acid sequence that hybridizes under high or medium stringency hybridization conditions to a nucleic acid sequence encoding a mammalian IL-15Ra polypeptide; (e) a nucleic acid sequence that hybridizes under high or medium stringency hybridization conditions to a fragment of a nucleic acid sequence encoding a mammalian IL-15Ra polypeptide; (f) a nucleic acid sequence encoding a fragment of a nucleic acid sequence encoding a mammalian IL-15Ra polypeptide; and/or (g) a nucleic acid sequence encoding a specific IL-15Ra derivative described herein. In a specific embodiment, an IL-15Ra derivative in the context of nucleic acids is a derivative of a nucleic acid sequence encoding a human IL-15Ra polypeptide. In another embodiment, an IL-15Ra derivative in the context of nucleic acids is a derivative of a nucleic acid sequence encoding an immature form of a human IL-15Ra polypeptide. In another embodiment, an IL-15Ra derivative in the context of nucleic acids is a derivative of a nucleic acid sequence encoding a mature form of a human IL-15Ra polypeptide. In one embodiment, an IL-15Ra derivative in the context of nucleic acids refers to a nucleic acid sequence encoding a derivative of mammalian IL-15Ra polypeptide that is soluble. In certain embodiments, an IL-15Ra derivative in context of nucleic acids refers to a nucleic acid sequence encoding a soluble form of mammalian IL-15Ra, wherein the soluble form is not naturally occurring. In some embodiments, an IL-15Ra derivative in the context of nucleic acids refers to a nucleic acid sequence encoding a derivative of human IL-15Ra, wherein the derivative of the human IL-15Ra is a soluble form of IL-15Ra that is not naturally occurring. In specific embodiments, an IL-15Ra derivative nucleic acid sequence is isolated or purified.

IL-15Ra derivative nucleic acid sequences include codon-optimized nucleic acid sequences that encode an IL-15Ra polypeptide, including mature and immature forms of IL-15Ra polypeptide. In other embodiments, IL-15Ra derivative nucleic acids include nucleic acids that encode IL-15Ra RNA transcripts containing mutations that eliminate potential splice sites and instability elements (e.g., A/T or A/U rich elements) without affecting the amino acid sequence to increase the stability of the IL-15Ra RNA transcripts. In certain embodiments, the IL-15Ra derivative nucleic acid sequence is the codon-optimized sequence in SEQ ID NO: 11, 13 in Table 1 (the amino acid sequences encoded by such a nucleic acid sequences are provided in SEQ ID NO: 12, 14 in Table 1, respectively).

In specific embodiments, IL-15Ra derivative nucleic acid sequences encode proteins or polypeptides that retain at least 50%, 55%, 60%, 65%, 70%, 75%, 80%, 85%, 90%, 95%, 98% or 99% of the function of a wild-type mammalian IL-15Ra polypeptide to bind IL-15, as measured by assays well known in the art, e.g., ELISA, BIAcore™, co-immunoprecipitation. In another preferred embodiment, IL-15Ra derivative nucleic acid sequences encode proteins or polypeptides that retain at least 50%, 55%, 60%, 65%, 70%, 75%, 80%, 85%, 90%, 95%, 98% or 99% of the function of a wild-type mammalian IL-15Ra to induce IL-15-mediated signal transduction, as measured by assays well-known in the art, e.g., electromobility shift assays, ELISAs and other immunoassays. In a specific embodiment, IL-15Ra derivative nucleic acid sequences encode proteins or polypeptides that bind to IL-15 as assessed by methods well-known in the art, such as, e.g., ELISAs.

Described herein is a soluble form of human IL-15Ra. Also described herein are specific IL-15Ra derivatives that are truncated, soluble forms of human IL-15Ra. These specific IL-15Ra derivatives and the soluble form of human IL-15Ra are based, in part, on the identification of the proteolytic cleavage site of human IL-15Ra. Further described herein are soluble forms of IL-15Ra that are characterized based upon glycosylation of the IL-15Ra.

The proteolytic cleavage of human IL-15Ra takes place between the residues (i.e., Glyl70 and His171) which are in shown in bold and underlined in the provided amino acid sequence of the immature form of the wild-type full length human IL-15Ra:

(SEQ ID NO: 6 in Table 1)
MAPRRARGCR TLGLPALLLL LLLRPPATRG ITCPPPMSVE
HADIWVKSYS LYSRERYICN SGFKRKAGTS SLTECVLNKA
TNVAHWTTPS LKCIRDPALV HQRPAPPSTV TTAGVTPQPE
SLSPSGKEPA ASSPSSNNTA ATTAAIVPGS QLMPSKSPST
GTTEISSHES SHGTPSQTTA KNWELTASAS HQPPGVYPQG
HSDTTVAIST STVLLCGLSA VSLLACYLKS RQTPPLASVE
MEAMEALPVT WGTSSRDEDL ENCSHHL.

Accordingly, in one aspect, provided herein is a soluble form of human IL-15Ra (e.g., a purified soluble form of human IL-15Ra), wherein the amino acid sequence of the soluble form of human IL-15Ra terminates at the site of the proteolytic cleavage of the wild-type membrane-bound human IL-15Ra. In particular, provided herein is a soluble form of human IL-15Ra (e.g., a purified soluble form of human IL-15Ra), wherein the amino acid sequence of the soluble form of human IL-15Ra terminates with PQG (SEQ ID NO: 20 in Table 1), wherein G is Gly170. In a particular embodiment, provided herein is a soluble form of human IL-15Ra (e.g., a purified soluble form of human IL-15Ra) which has the amino acid sequence shown in SEQ ID NO: 7 in Table 1. In some embodiments, provided herein is an IL-15Ra derivative (e.g., a purified and/or soluble form of IL-15Ra derivative), which is a polypeptide that: (i) is at least 75%, 80%, 85%, 90%, 95%, 98% or 99% identical to SEQ ID NO: 7 in Table 1; and (ii) terminates with the amino acid sequence PQG (SEQ ID NO: 20 in Table 1). In other particular embodiments, provided herein is a soluble form of human IL-15Ra (e.g., a purified soluble form of human IL-15Ra) which has the amino acid sequence of SEQ ID NO: 10 in Table 1). In some embodiments, provided herein is an IL-15Ra derivative (e.g., a purified and/or soluble form of an IL-15Ra derivative), which is a polypeptide that is at least 75%, 80%, 85%, 90%, 95%, 98% or 99% identical to SEQ ID NO: 10 in Table 1, and, optionally, wherein the amino acid sequence of the soluble form of the IL-15Ra derivative terminates with PQG (SEQ ID NO: 20 in Table 1).

In some embodiments, provided herein is an IL-15Ra derivative of human IL-15Ra, wherein the IL-15Ra derivative is soluble and: (a) the last amino acids at the C-terminal end of the IL-15Ra derivative consist of amino acid residues PQGHSDTT (SEQ ID NO: 15 in Table 1); (b) the last amino acids at the C-terminal end of the IL-15Ra derivative consist of amino acid residues PQGHSDT (SEQ ID NO: 16 in Table 1); (c) the last amino acids at the C-terminal end of the IL-15Ra derivative consist of amino acid residues PQGHSD (SEQ ID NO: 17 in Table 1); (d) the last amino acids at the C-terminal end of the IL-15Ra derivative consist of amino acid residues PQGHS (SEQ ID NO: 18 in Table 1); or (e) the last amino acids at the C-terminal end of the IL-15Ra derivative consist of amino acid residues PQGH (SEQ ID NO: 19 in Table 1). In certain embodiments, the amino acid sequences of these IL-15Ra derivatives are at least 75%, at least 85%, at least 90%, at least 95%, at least 96%, at least 97%, at least 98%, or at least 99% identical to the amino acid sequence of SEQ ID NO: 21 in Table 1. In some embodiments, these IL-15Ra derivatives are purified.

In another aspect, provided herein are glycosylated forms of IL-15Ra (e.g., purified glycosylated forms of IL-15Ra), wherein the glycosylation of the IL-15Ra accounts for at least 20%, at least 25%, at least 30%, at least 35%, at least 40%, at least 45%, at least 50%, or 20% to 25%, 20% to 30%, 25% to 30%, 25% to 35%, 30% to 35%, 30% to 40%, 35% to 40%, 35% to 45%, 40% to 50%, 45% to 50%, 20% to 40%, or 25% to 50% of the mass (molecular weight) of the IL-15Ra as assessed by techniques known to one of skill in the art. The percentage of the mass (molecular weight) of IL-15Ra (e.g., purified IL-15Ra) that glycosylation of IL-15Ra accounts for can be determined using, for example and without limitation, gel electrophoresis and quantitative densitometry of the gels, and comparison of the average mass (molecular weight) of a glycosylated form of IL-15Ra (e.g., a purified glycosylated form of IL-15Ra) to the non-glycosylated form of IL-15Ra (e.g., a purified non-glycosylated form of IL-15Ra). In one embodiment, the average mass (molecular weight) of IL-15Ra (e.g., purified IL-15Ra) can be determined using MALDI-TOF MS spectrum on Voyager De-Pro equipped with CovalX HM-1 high mass detector using sinapic acid as matrix, and the mass of a glycosylated form of IL-15Ra (e.g., purified glycosylated form of IL-15Ra) can be compared to the mass of the non-glycosylated form of IL-15Ra (e.g., purified non-glycosylated form of IL-15Ra) to determine the percentage of the mass that glycosylation accounts for.

In another aspect, provided herein are glycosylated forms of IL-15Ra, wherein the IL-15Ra is glycosylated (N- or O-glycosylated) at certain amino acid residues. In certain embodiments, provided herein is a human IL-15Ra which is glycosylated at one, two, three, four, five, six, seven, or all, of the following glycosylation sites: (i) 0-glycosylation on threonine at position 5 of the amino acid sequence NWELTASASHQPPGVYPQG (SEQ ID NO: 22 in Table 1) in the IL-15Ra; (ii) 0-glycosylation on serine at position 7 of the amino acid sequence NWELTASASHQPPGVYPQG (SEQ ID NO: 22 in Table 1) in the IL-15Ra; (iii) N-glycosylation on serine at position 8 of the amino acid sequence ITCPPPMSVEHADIWVK (SEQ ID NO: 23 in Table 1) in the IL-15Ra, or serine at position 8 of the amino acid sequence ITCPPPMSVEHADIWVKSYSLYSRERYICNS (SEQ ID NO: 24 in Table 1) in the IL-15Ra; (iv) N-glycosylation on Ser 18 of amino acid sequence ITCPPPMSVEHADIWVKSYSLYSRERYICNS (SEQ ID NO: 24 in Table 1) in the IL-15Ra; (v) N-glycosylation on serine at position 20 of the amino acid sequence ITCPPPMSVEHADIWVKSYSLYSRERYICNS (SEQ ID NO: 24 in Table 1) in the IL-15Ra; (vi) N-glycosylation on serine at position 23 of the amino acid sequence ITCPPPMSVEHADIWVKSYSLYSRERYICNS (SEQ ID NO: 24 in Table 1) in the IL-15Ra; and/or (vii) N-glycosylated on serine at position 31 of the amino acid sequence ITCPPPMSVEHADIWVKSYSLYSRERYICNS (SEQ ID NO: 24 in Table 1) in the IL-15Ra.

In specific embodiments, the glycosylated IL-15Ra is a wild-type human IL-15Ra. In other specific embodiments, the glycosylated IL-15Ra is an IL-15Ra derivative of human IL-15Ra. In some embodiments, the glycosylated IL-15Ra is a wild-type soluble human IL-15Ra, such as SEQ ID NO: 7 or 10 in Table 1. In other embodiments, the glycosylated IL-15Ra is an IL-15Ra derivative that is a soluble form of human IL-15Ra. In certain embodiments, the glycosylated IL-15Ra is purified or isolated.

IL-15/IL-15Ra complex

As used herein, the term “IL-15/IL-15Ra complex” refers to a complex comprising IL-15 and IL-15Ra covalently or noncovalently bound to each other. In a preferred embodiment, the IL-15Ra has a relatively high affinity for IL-15, e.g., K_{D} of 10 to 50 μM as measured by a technique known in the art, e.g., KinEx A assay, plasma surface resonance (e.g., BIAcore™ assay). In another preferred embodiment, the IL-15/IL-15Ra complex induces IL-15-mediated signal transduction, as measured by assays well-known in the art, e.g., electromobility shift assays, ELISAs and other immunoassays. In some embodiments, the IL-15/IL-15Ra complex retains the ability to specifically bind to the βγ chain. In a specific embodiment, the IL-15/IL-15Ra complex is isolated from a cell.

Provided herein are complexes that bind to the βγ subunits of the IL-15 receptor, induce IL-15 signal transduction (e.g., Jak/Stat signal transduction) and enhance IL-15-mediated immune function, wherein the complexes comprise IL-15 covalently or noncovalently bound to interleukin-15 receptor alpha (“IL-15Ra”) (a “IL-15/IL-15Ra complex”). The IL-15/IL-15Ra complex is able to bind to the βγ receptor complex.

The IL-15/IL-15Ra complexes may be composed of wild-type IL-15 or an IL-15 derivative and wild-type IL-15Ra or an IL-15Ra derivative. In certain embodiments, an IL-15/IL-15Ra complex comprises IL-15 or an IL-15 derivative and an IL-15Ra described above. In a specific embodiment, an IL-15/IL-15Ra complex comprises IL-15 or an IL-15 derivative and IL-15Ra with the amino acid sequence of SEQ ID NO: 10 in Table 1. In another embodiment, an IL-15/IL-15Ra complex comprises IL-15 or an IL-15 derivative and a glycosylated form of IL-15Ra described supra.

In a specific embodiment, an IL-15/IL-15Ra complex comprises wild-type IL-15 or an IL-15Ra derivative and soluble IL-15Ra (e.g., wild-type soluble human IL-15Ra). In another specific embodiment, an IL-15/IL-15Ra complex is composed of an IL-15 derivative and an IL-15Ra derivative. In another embodiment, an IL-15/IL-15Ra complex is composed of wild-type IL-15 and an IL-15Ra derivative. In one embodiment, the IL-15Ra derivative is a soluble form of IL-15Ra. Specific examples of soluble forms of IL-15Ra are described above. In a specific embodiment, the soluble form of IL-15Ra lacks the transmembrane domain of wild-type IL-15Ra, and optionally, the intracellular domain of wild-type IL-15Ra. In another embodiment, the IL-15Ra derivative is the extracellular domain of wild-type IL-15Ra or a fragment thereof. In certain embodiments, the IL-15Ra derivative is a fragment of the extracellular domain comprising the sushi domain or exon 2 of wild-type IL-15Ra. In some embodiments, the IL-15Ra derivative comprises a fragment of the extracellular domain comprising the sushi domain or exon 2 of wild-type IL-15Ra and at least one amino acid that is encoded by exon 3. In certain embodiments, the IL-15Ra derivative comprises a fragment of the extracellular domain comprising the sushi domain or exon 2 of wild-type IL-15Ra and an IL-15Ra hinge region or a fragment thereof. In certain embodiments, the IL-15Ra comprises the amino acid sequence of SEQ ID NO: 10 in Table 1.

In another embodiment, the IL-15Ra derivative comprises a mutation in the extracellular domain cleavage site that inhibits cleavage by an endogenous protease that cleaves wild-type IL-15Ra. In a specific embodiment, the extracellular domain cleavage site of IL-15Ra is replaced with a cleavage site that is recognized and cleaved by a heterologous known protease. Non-limiting examples of such heterologous protease cleavage sites include Arg-X-X-Arg (SEQ ID NO: 25 in Table 1), which is recognized and cleaved by furin protease; and A-B-Pro-Arg-X-Y (SEQ ID NO: 26 in Table 1) (A and B are hydrophobic amino acids and X and Y are non-acidic amino acids) and Gly-Arg-Gly, which are recognized and cleaved by thrombin protease.

In another embodiment, the IL-15 is encoded by a nucleic acid sequence optimized to enhance expression of IL-15, e.g., using methods as described in WO 2007/084342 and WO 2010/020047; and U.S. Pat. Nos. 5,965,726; 6,174,666; 6,291,664; 6,414,132; and 6,794,498.

In certain embodiments, provided herein is an IL-15/IL-15Ra complex comprising human IL-15Ra which is glycosylated at one, two, three, four, five, six, seven, or all, of the glycosylation sites as described supra and with reference to SEQ ID NOs: 22, 23 and 24 in Table 1. In specific embodiments, the glycosylated IL-15Ra is a wild-type human IL-15Ra. In other specific embodiments, the glycosylated IL-15Ra is an IL-15Ra derivative of human IL-15Ra. In some embodiments, the glycosylated IL-15Ra is a wild-type soluble human IL-15Ra, such as SEQ ID NO: 7 or 10 in Table 1. In other embodiments, the glycosylated IL-15Ra is an IL-15Ra derivative that is a soluble form of human IL-15Ra. In certain embodiments, the IL-15/IL-15Ra complex is purified or isolated.

In addition to IL-15 and IL-15Ra, the IL-15/IL-15Ra complexes may comprise a heterologous molecule. In some embodiments, the heterologous molecule increases protein stability. Non-limiting examples of such molecules include polyethylene glycol (PEG), Fc domain of an IgG immunoglobulin or a fragment thereof, or albumin that increase the half-life of IL-15 or IL-15Ra in vivo. In certain embodiments, IL-15Ra is conjugated/fused to the Fc domain of an immunoglobulin (e.g., an IgG1) or a fragment thereof. In a specific embodiment, the IL-15RaFc fusion protein comprises the amino acid sequence of SEQ ID NO: 27 or 28 in Table 1. In another embodiment, the IL-15RaFc fusion protein is the IL-15Ra/Fc fusion protein described in Han et al., (2011), Cytokine 56: 804-810, U.S. Pat. Nos. 8,507,222 or 8,124,084. In those IL-15/IL-15Ra complexes comprising a heterologous molecule, the heterologous molecule may be conjugated to IL-15 and/or IL-15Ra. In one embodiment, the heterologous molecule is conjugated to IL-15Ra. In another embodiment, the heterologous molecule is conjugated to IL-15.

The components of an IL-15/IL-15Ra complex may be directly fused, using either non-covalent bonds or covalent bonds (e.g., by combining amino acid sequences via peptide bonds), and/or may be combined using one or more linkers. Linkers suitable for preparing the IL-15/IL-15Ra complexes comprise peptides, alkyl groups, chemically substituted alkyl groups, polymers, or any other covalently-bonded or non-covalently bonded chemical substance capable of binding together two or more components. Polymer linkers comprise any polymers known in the art, including polyethylene glycol (PEG). In some embodiments, the linker is a peptide that is 1, 2, 3, 4, 5, 6, 7, 8, 9, 10, 11, 12, 13, 14, 15, 16, 17, 18, 19, 20 or more amino acids long. In a specific embodiment, the linker is long enough to preserve the ability of IL-15 to bind to the IL-15Ra. In other embodiments, the linker is long enough to preserve the ability of the IL-15/IL-15Ra complex to bind to the 13y receptor complex and to act as an agonist to mediate IL-15 signal transduction.

In particular embodiments, IL-15/IL-15Ra complexes are pre-coupled prior to use in the methods described herein (e.g., prior to contacting cells with the IL-15/IL-15Ra complexes or prior to administering the IL-15/IL-15Ra complexes to a subject). In other embodiments, the IL-15/IL-15Ra complexes are not pre-coupled prior to use in the methods described herein.

In a specific embodiment, an IL-15/IL-15Ra complex enhances or induces immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the IL-15/IL-15Ra complex using assays well known in the art, e.g., ELISPOT, ELISA, and cell proliferation assays. In a specific embodiment, the immune function is cytokine release (e.g., interferon-gamma, IL-2, IL-5, IL-10, IL-12, or transforming growth factor (TGF) -beta). In one embodiment, the IL-15 mediated immune function is NK cell proliferation, which can be assayed, e.g., by flow cytometry to detect the number of cells expressing markers of NK cells (e.g., CD56). In another embodiment, the IL-15 mediated immune function is antibody production, which can be assayed, e.g., by ELISA. In some embodiments, the IL-15 mediated immune function is effector function, which can be assayed, e.g., by a cytotoxicity assay or other assays well known in the art.

In specific embodiments, examples of immune function enhanced by the IL-15/IL-15Ra complex include the proliferation/expansion of lymphocytes (e.g., increase in the number of lymphocytes), inhibition of apoptosis of lymphocytes, activation of dendritic cells (or antigen presenting cells), and antigen presentation. In particular embodiments, an immune function enhanced by the IL-15/IL-15Ra complex is proliferation/expansion in the number of or activation of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells). In one embodiment, the IL-15/IL-15Ra complex enhances the proliferation/expansion or number of lymphocyte progenitors. In some embodiments, a IL-15/IL-15Ra complex increases the number of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells) by approximately 1 fold, 2 fold, 3 fold, 4 fold, 5 fold, 6 fold, 7 fold, 8 fold, 9 fold, 10 fold, 20 fold, or more relative a negative control (e.g., number of the respective cells not treated, cultured, or contacted with an IL-15/IL-15Ra complex).

In a specific embodiment, the IL-15/IL-15Ra complex increases the expression of IL-2 on whole blood activated by Staphylococcal enterotoxin B (SEB). For example, the IL-15/IL-15Ra complex increases the expression of IL-2 by at least about 2, 3, 4, or 5-fold, compared to the expression of IL-2 when SEB alone is used.

Anti-PD-1 Antibody Molecules

The anti-PD-1 antibody molecules described herein can be used alone or in combination with one or more additional agents described herein in accordance with a method described herein. In certain embodiments, the combinations described herein include a PD-1 inhibitor, e.g., an anti-PD-1 antibody molecule (e.g., humanized antibody molecules) as described herein.

In some embodiments, the anti-PD-1 antibody molecule (e.g., an isolated or recombinant antibody molecule) has one or more of the following properties:

(i) binds to PD-1, e.g., human PD-1, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger;

(ii) does not substantially bind to CD28, CTLA-4, ICOS or BTLA;

(iii) inhibits or reduces binding of PD-1 to a PD-1 ligand, e.g., PD-L1 or PD-L2, or both;

(iv) binds specifically to an epitope on PD-1, e.g., the same or similar epitope as the epitope recognized by antibody BAP049-Clone-B or BAP049-Clone-E as described in Table 1;

(v) shows the same or similar binding affinity or specificity, or both, as BAP049-Clone-B or BAP049-Clone-E as described in Table 1;

(vi) shows the same or similar binding affinity or specificity, or both, as an antibody molecule e.g., a heavy chain variable region and light chain variable region having amino acid sequences of SEQ ID Nos: 35 and 45 or SEQ ID NOs: 55 and 65 in Table 1;

(vii) shows the same or similar binding affinity or specificity, or both, as an antibody molecule e.g., an heavy chain variable region and light chain variable region encoded by the nucleotide sequences SEQ ID Nos: 36 and 46 or SEQ ID Nos: 56 and 66 in Table 1;

(viii) shows the same or similar binding affinity or specificity, or both, as an antibody molecule e.g., a heavy chain and light chain having amino acid sequences of SEQ ID NOs: 37 and 47 or SEQ ID NOs: 57 and 67 in Table 1;

(ix) inhibits, e.g., competitively inhibits, the binding of a second antibody molecule to PD-1, wherein the second antibody molecule is an antibody molecule described herein, e.g., an antibody molecule chosen from, e.g., BAP049-Clone-B or BAP049-Clone-E as described in Table 1;

(x) binds the same or an overlapping epitope with a second antibody molecule to PD-1, wherein the second antibody molecule is an antibody molecule described herein, e.g., an antibody molecule chosen from, e.g., BAP049-Clone-B or BAP049-Clone-E as described in Table 1;

(xi) competes for binding, and/or binds the same epitope, with a second antibody molecule to PD-1, wherein the second antibody molecule is an antibody molecule described herein, e.g., an antibody molecule chosen from, e.g., BAP049-Clone-B or BAP049-Clone-E as described in Table 1;

(xii) has one or more biological properties of an antibody molecule described herein, e.g., an antibody molecule chosen from, e.g., BAP049-Clone-B or BAP049-Clone-E as described in Table 1;

(xiii) has one or more pharmacokinetic properties of an antibody molecule described herein, e.g., an antibody molecule chosen from, e.g. BAP049-Clone-B or BAP049-Clone-E as described in Table 1;

(xiv) inhibits one or more activities of PD-1, e.g., results in one or more of: an increase in tumor infiltrating lymphocytes, an increase in T-cell receptor mediated proliferation, or a decrease in immune evasion by cancerous cells;

(xv) binds human PD-1 and is cross-reactive with cynomolgus PD-1;

(xvi) binds to one or more residues within the C strand, CC' loop, C′ strand, or FG loop of PD-1, or a combination two, three or all of the C strand, CC′ loop, C′ strand or FG loop of PD-1, e.g., wherein the binding is assayed using ELISA or BIAcore™; or

(xvii) has a VL region that contributes more to binding to PD-1 than a VH region.

In some embodiments, the antibody molecule binds to PD-1 with high affinity, e.g., with a KD that is about the same, or at least about 10%, 20%, 30%, 40%, 50%, 60%, 70%, 80% or 90% higher or lower than the KD of a murine or chimeric anti-PD-1 antibody molecule, e.g., a murine or chimeric anti-PD-1 antibody molecule described herein. In some embodiments, the KD of the murine or chimeric anti-PD-1 antibody molecule is less than about 0.4, 0.3, 0.2, 0.1, or 0.05 nM, e.g., measured by a BIAcore™ method. In some embodiments, the K_{D} of the murine or chimeric anti-PD-1 antibody molecule is less than about 0.2 nM, e.g., about 0.135 nM. In other embodiments, the K_{D} of the murine or chimeric anti PD-1 antibody molecule is less than about 10, 5, 3, 2, or 1 nM, e.g., measured by binding on cells expressing PD-1 (e.g., 300.19 cells). In some embodiments, the K_{D} of the murine or chimeric anti PD-1 antibody molecule is less than about 5 nM, e.g., about 4.60 nM (or about 0.69 μg/mL).

In some embodiments, the anti-PD-1 antibody molecule binds to PD-1 with a K_{off} slower than 1×10 ⁴, 5×10⁻⁵, or 1×10⁻⁵ s⁻¹, e.g., about 1.65×10⁻⁵ s⁻¹. In some embodiments, the anti-PD-1 antibody molecule binds to PD-1 with a Kₒₙ faster than 1×10⁴, 5×10⁴, 1×10⁵, or 5×10⁵ M⁻¹s⁻¹, e.g., about 1.23×10⁵ M⁻¹s⁻¹.

In some embodiments, the expression level of the antibody molecule is higher, e.g., at least about 0.5, 1, 2, 3, 4, 5, 6, 7, 8, 9 or 10-fold higher, than the expression level of a murine or chimeric antibody molecule, e.g., a murine or chimeric anti-PD-1 antibody molecule described herein. In some embodiments, the antibody molecule is expressed in CHO cells.

In some embodiments, the anti-PD-1 antibody molecule reduces one or more PD-1-associated activities with an IC50 (concentration at 50% inhibition) that is about the same or lower, e.g., at least about 10%, 20%, 30%, 40%, 50%, 60%, 70%, 80% or 90% lower, than the IC50 of a murine or chimeric anti-PD-1 antibody molecule, e.g., a murine or chimeric anti-PD-1 antibody molecule described herein. In some embodiments, the IC50 of the murine or chimeric anti-PD-1 antibody molecule is less than about 6, 5, 4, 3, 2, or 1 nM, e.g., measured by binding on cells expressing PD-1 (e.g., 300.19 cells). In some embodiments, the IC₅₀ of the murine or chimeric anti-PD-1 antibody molecule is less than about 4 nM, e.g., about 3.40 nM (or about 0.51 μg/mL). In some embodiments, the PD-1-associated activity reduced is the binding of PD-L1 and/or PD-L2 to PD-1. In some embodiments, the anti-PD-1 antibody molecule binds to peripheral blood mononucleated cells (PBMCs) activated by Staphylococcal enterotoxin B (SEB). In other embodiments, the anti-PD-1 antibody molecule increases the expression of IL-2 on whole blood activated by SEB. For example, the anti-PD-1 antibody increases the expression of IL-2 by at least about 2, 3, 4, or 5-fold, compared to the expression of IL-2 when an isotype control (e.g., IgG4) is used.

In some embodiments, the anti-PD-1 antibody molecule has improved stability, e.g., at least about 0.5, 1, 2, 3, 4, 5, 6, 7, 8, 9 or 10-fold more stable in vivo or in vitro, than a murine or chimeric anti-PD-1 antibody molecule, e.g., a murine or chimeric anti-PD-1 antibody molecule described herein.

In one embodiment, the anti PD-1 antibody molecule is a humanized antibody molecule and has a risk score based on T cell epitope analysis of 300 to 700, 400 to 650, 450 to 600, or a risk score as described herein.

In yet another embodiment, the anti-PD-1 antibody molecule comprises at least one, two, three or four variable regions from an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two heavy chain variable regions from an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two light chain variable regions from an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes a heavy chain constant region for an IgG4, e.g., a human IgG4. In one embodiment, the human IgG4 includes a substitution at position 228 according to EU numbering (e.g., a Ser to Pro substitution). In still another embodiment, the anti-PD-1 antibody molecule includes a heavy chain constant region for an IgG1, e.g., a human IgG1. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235). In one embodiment, the heavy chain constant region comprises an amino sequence set forth in SEQ ID No: 98 to 103 in Table 1, or a sequence substantially identical (e.g., at least 80%, 85%, 90%, 92%, 95%, 97%, 98%, 99% or higher identical) thereto.

In yet another embodiment, the anti-PD-1 antibody molecule includes a kappa light chain constant region, e.g., a human kappa light chain constant region. In one embodiment, the light chain constant region comprises an amino sequence set forth in SEQ ID NO: 104 in Table 1, or a sequence substantially identical (e.g., at least 80%, 85%, 90%, 92%, 95%, 97%, 98%, 99% or higher identical) thereto.

In another embodiment, the anti-PD-1 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 1, or a sequence substantially identical (e.g., at least 80%, 85%, 90%, 92%, 95%, 97%, 98%, 99% or higher identical) thereto. In one embodiment, the human IgG4 includes a substitution at position 228 according to EU numbering (e.g., a Ser to Pro substitution). In yet another embodiment, the anti-PD-1 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 1, or a sequence substantially identical (e.g., at least 80%, 85%, 90%, 92%, 95%, 97%, 98%, 99% or higher identical) thereto. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235).

In another embodiment, the anti-PD-1 antibody molecule includes a heavy chain variable domain and a constant region, a light chain variable domain and a constant region, or both, comprising the amino acid sequence of BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, or a sequence substantially identical thereto.

In yet another embodiment, the anti-PD-1 antibody molecule includes at least one, two, or three complementarity determining regions (CDRs) from a heavy chain variable region of an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 1, or encoded by a nucleotide sequence shown in Table 1. In one embodiment, one or more of the CDRs (or collectively all of the CDRs) have one, two, three, four, five, six or more changes, e g , amino acid substitutions or deletions, relative to the amino acid sequence shown in Table 1, or encoded by a nucleotide sequence shown in Table 1.

In yet another embodiment, the anti-PD-1 antibody molecule includes at least one, two, or three CDRs from a light chain variable region of an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequence.

In yet another embodiment, the anti-PD-1 antibody molecule includes at least one, two, or three CDRs (or collectively all of the CDRs) from a light chain variable region comprising an amino acid sequence shown in Table 1, or encoded by a nucleotide sequence shown in Table 1. In one embodiment, one or more of the CDRs (or collectively all of the CDRs) have one, two, three, four, five, six or more changes, e g , amino acid substitutions or deletions, relative to the amino acid sequence shown in Table 1, or encoded by a nucleotide sequence shown in Table 1.

In one embodiment, the anti-PD-1 antibody molecule includes all six CDRs from an antibody described herein, e.g., an antibody chosen from BAP049-Clone-B or BAP049-Clone-E; as described in Table 1, or encoded by the nucleotide sequence in Table 1, or closely related CDRs, e.g., CDRs which are identical or which have at least one amino acid alteration, but not more than two, three or four alterations (e.g., substitutions, deletions, or insertions, e.g., conservative substitutions). In one embodiment, the anti-PD-1 antibody molecule may include any CDR described herein. In certain embodiments, the anti-PD-1 antibody molecule includes a substitution in a light chain CDR, e.g., one or more substitutions in a CDR1, CDR2 and/or CDR3 of the light chain.

In another embodiment, the anti-PD-1 antibody molecule includes at least one, two, or three CDRs according to Kabat et al. (e.g., at least one, two, or three CDRs according to the Kabat definition as set out in Table 1) from a heavy chain variable region of an antibody described herein, e.g., an antibody chosen from any of BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In another embodiment, the anti-PD-1 antibody molecule includes at least one, two, or three CDRs according to Kabat et al. (e.g., at least one, two, or three CDRs according to the Kabat definition as set out in Table 1) from a light chain variable region of an antibody described herein, e.g., an antibody chosen from any of BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 1.

In yet another embodiment, the anti-PD-1 antibody molecule includes at least one, two, three, four, five, or six CDRs according to Kabat et al. (e.g., at least one, two, three, four, five, or six CDRs according to the Kabat definition as set out in Table 1) from the heavy and light chain variable regions of an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 1.

In yet another embodiment, the anti-PD-1 antibody molecule includes all six CDRs according to Kabat et al. (e.g., all six CDRs according to the Kabat definition as set out in Table 1) from the heavy and light chain variable regions of an antibody described herein, e.g., an antibody chosen from BAP049-Clone-B or BAP049-Clone-E as described in Table 1, or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 1. In one embodiment, the anti-PD-1 antibody molecule may include any CDR described herein.

In another embodiment, the anti-PD-1 antibody molecule includes at least one, two, or three Chothia hypervariable loops (e.g., at least one, two, or three hypervariable loops according to the Chothia definition as set out in Table 1) from a heavy chain variable region of an antibody described herein, e.g., an antibody chosen from BAP049-Clone-B or BAP049-Clone-E as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

In another embodiment, the anti-PD-1 antibody molecule includes at least one, two, or three Chothia hypervariable loops (e.g., at least one, two, or three hypervariable loops according to the Chothia definition as set out in Table 1) of a light chain variable region of an antibody described herein, e.g., an antibody chosen from BAP049-Clone-B or BAP049-Clone-E as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 1.

In yet another embodiment, the anti-PD-1 antibody molecule includes at least one, two, three, four, five, or six hypervariable loops (e.g., at least one, two, three, four, five, or six hypervariable loops according to the Chothia definition as set out in Table 1) from the heavy and light chain variable regions of an antibody described herein, e.g., an antibody chosen from BAP049-Clone-B or BAP049-Clone-E as described in Table 1, or encoded by the nucleotide sequence in Table 1; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 1.

In one embodiment, the anti-PD-1 antibody molecule includes all six hypervariable loops (e.g., all six hypervariable loops according to the Chothia definition as set out in Table 1) of an antibody described herein, e.g., an antibody chosen from BAP049-Clone-B or BAP049-Clone-E, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 1. In one embodiment, the anti-PD-1 antibody molecule may include any hypervariable loop described herein.

In still another embodiment, the anti-PD-1 antibody molecule includes at least one, two, or three hypervariable loops that have the same canonical structures as the corresponding hypervariable loop of an antibody described herein, e.g., an antibody chosen from BAP049-Clone-B or BAP049-Clone-E, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-PD-1 antibody molecule includes a combination of CDRs or hypervariable loops defined according to the Kabat et al or Chothia et al.

In one embodiment, the anti-PD-1 antibody molecule includes at least one, two or three CDRs or hypervariable loops from a heavy chain variable region of an antibody described herein, e.g., an antibody chosen from BAP049-Clone-B or BAP049-Clone-E as shown in Table 1, according to the Kabat or Chothia definitions (e.g., at least one, two, or three CDRs or hypervariable loops according to the Kabat or Chothia definitions as set out in Table 1); or encoded by the nucleotide sequence in Table 1; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat or Chothia shown in Table 1.

For example, the anti-PD-1 antibody molecule can include VH CDR1 according to Kabat et al. or VH hypervariable loop 1 according to Chothia et al., or a combination thereof, e.g., as shown in Table 1. In one embodiment, the combination of Kabat and Chothia CDR of VH CDR1 comprises the amino acid sequence GYTFTTYWMH (SEQ ID NO: 93-), or an amino acid sequence substantially identical thereto (e.g., having at least one amino acid alteration, but not more than two, three or four alterations (e.g., substitutions, deletions, or insertions, e.g., conservative substitutions)). The anti-PD-1 antibody molecule can further include, e.g., VH CDRs 2-3 according to Kabat et al. and VL CDRs 1-3 according to Kabat et al., e.g., as shown in Table 1. Accordingly, in some embodiments, framework regions are defined based on a combination of CDRs defined according to Kabat et al. and hypervariable loops defined according to Chothia et al. For example, the anti-PD-1 antibody molecule can include VH FR1 defined based on VH hypervariable loop 1 according to Chothia et al. and VH FR2 defined based on VH CDRs 1-2 according to Kabat et al.. The anti-PD-1 antibody molecule can further include, e.g., VH FRs 3-4 defined based on VH CDRs 2-3 according to Kabat et al. and VL FRs 1-4 defined based on VL CDRs 1-3 according to Kabat et al.

The anti-PD-1 antibody molecule can contain any combination of CDRs or hypervariable loops according to the Kabat and Chothia definitions. In one embodiment, the anti-PD-1 antibody molecule includes at least one, two or three CDRs from a light chain variable region of an antibody described herein, e.g., an antibody chosen from any of BAP049-Clone-B or BAP049-Clone-E, according to the Kabat and Chothia definition (e.g., at least one, two, or three CDRs according to the Kabat and Chothia definition as set out in Table 1).

In the combinations herein, in another embodiment, the anti-PD-1 antibody molecule comprises (i) a heavy chain variable region (VH) comprising a VHCDR1 amino acid sequence chosen from SEQ ID NO: 29, SEQ ID NO: 32, or SEQ ID NO: 93; a VHCDR2 amino acid sequence of SEQ ID NO: 30 or SEQ ID NO: 33; and a VHCDR3 amino acid sequence of SEQ ID NO: 31; and (ii) a light chain variable region (VL) comprising a VLCDR1 amino acid sequence of SEQ ID NO: 39 or SEQ ID NO: 42, a VLCDR2 amino acid sequence of SEQ ID NO: 40 or SEQ ID NO: 43, and a VLCDR3 amino acid sequence of SEQ ID NO: 41 or SEQ ID NO: 44.

In one embodiment, the anti-PD-1 antibody molecule includes: a heavy chain variable region (VH) comprising a VHCDR1 amino acid sequence of SEQ ID NO: 29, a VHCDR2 amino acid sequence of SEQ ID NO: 30, and a VHCDR3 amino acid sequence of SEQ ID NO: 31; and a light chain variable region (VL) comprising a VLCDR1 amino acid sequence of SEQ ID NO: 39, a VLCDR2 amino acid sequence of SEQ ID NO: 40, and a VLCDR3 amino acid sequence of SEQ ID NO: 41.

In one embodiment, the anti-PD-1 antibody molecule includes: a heavy chain variable region (VH) comprising a VHCDR1 amino acid sequence of SEQ ID NO: 32, a VHCDR2 amino acid sequence of SEQ ID NO: 33, and a VHCDR3 amino acid sequence of SEQ ID NO: 34; and a light chain variable region (VL) comprising a VLCDR1 amino acid sequence of SEQ ID NO: 42, a VLCDR2 amino acid sequence of SEQ ID NO: 43, and a VLCDR3 amino acid sequence of SEQ ID NO: 44.

In one embodiment, the anti-PD-1 antibody molecule includes: a heavy chain variable region (VH) comprising a VHCDR1 amino acid sequence of SEQ ID NO: 93, a VHCDR2 amino acid sequence of SEQ ID NO: 33, and a VHCDR3 amino acid sequence of SEQ ID NO: 34; and a light chain variable region (VL) comprising a VLCDR1 amino acid sequence of SEQ ID NO: 42, a VLCDR2 amino acid sequence of SEQ ID NO: 43, and a VLCDR3 amino acid sequence of SEQ ID NO: 44; or

In one embodiment, the anti-PD-1 antibody molecule includes: a heavy chain variable region (VH) comprising a VHCDR1 amino acid sequence of SEQ ID NO: 93; a VHCDR2 amino acid sequence of SEQ ID NO: 30; and a VHCDR3 amino acid sequence of SEQ ID NO: 31; and a light chain variable region (VL) comprising a VLCDR1 amino acid sequence of SEQ ID NO: 39, a VLCDR2 amino acid sequence of SEQ ID NO: 40, and a VLCDR3 amino acid sequence of SEQ ID NO: 41.

Constructs & Cells

The nucleic acids encoding IL-15 and/or IL-15Ra and/or an anti-PD-1 antibody molecule can be inserted into nucleic acid constructs for expression in mammalian cells, bacteria, yeast, and viruses. IL-15 and IL-15Ra can be recombinantly expressed from the same nucleic acid construct (e.g., using a bicistronic nucleic acid construct) or from different nucleic acid constructs (e.g., using monocistronic nucleic acid constructs). In one embodiment, IL-15 and IL-15Ra can be recombinantly expressed from a single nucleic acid construct comprising a single open reading frame (ORF) of IL-15 and IL-15Ra.

The nucleic acid constructs may comprise one or more transcriptional regulatory element(s) operably linked to the coding sequence of IL-15 and/or IL-15Ra and/or an anti-PD-1 antibody molecule. The transcriptional regulatory elements are typically 5′ to the coding sequence and direct the transcription of the nucleic acids encoding IL-15 and/or IL-15Ra and/or an anti-PD-1 antibody molecule. In some embodiments, one or more of the transcriptional regulatory elements that are found in nature to regulate the transcription of the wild-type IL-15 and/or wild-type IL-15Ra gene are used to control transcription. In other embodiments, one or more transcriptional regulatory elements that are heterologous to the wild-type IL-15 and/or wild-type IL-15Ra gene are used to control transcription. Any transcriptional regulatory element(s) known to one of skill in the art may be used. Non-limiting examples of the types of transcriptional regulatory element(s) include a constitutive promoter, a tissue-specific promoter, and an inducible promoter. In a specific embodiment, transcription is controlled, at least in part, by a mammalian (in some embodiments, human) transcriptional regulatory element(s). In a specific embodiment, transcription is controlled, at least in part, by a strong promoter, e.g., CMV. In other aspects, an inducible promoter can be used.

The nucleic acid constructs also may comprise one or more post-transcriptional regulatory element(s) operably linked to the coding sequence of IL-15 and/or IL-15Ra and/or an anti-PD-1 antibody molecule. The post-transcriptional regulatory elements can be 5′ and/or 3′ to the coding sequence and direct the post-transcriptional regulation of the translation of RNA transcripts encoding IL-15 and/or IL-15Ra and/or an anti-PD-1 antibody molecule.

In another aspect, the nucleic acid construct can be a gene targeting vector that replaces a gene's existing regulatory region with a regulatory sequence isolated from a different gene or a novel regulatory sequence as described, e.g., in International Publication Nos. WO1994/12650 and WO2001/68882. In certain embodiments, a host cell can be engineered to increase production of endogenous IL-15 and/or IL-15Ra by, e.g., altering the regulatory region of the endogenous IL-15 and/or IL-15Ra genes.

The nucleic acid construct chosen will depend upon a variety of factors, including, without limitation, the strength of the transcriptional regulatory elements and the host cell to be used to express IL-15 and/or IL-15Ra and/or an anti-PD-1 antibody molecule. The nucleic acid constructs can be a plasmid, phagemid, cosmid, viral vector, phage, artificial chromosome, and the like. In one aspect, the vectors can be episomal or non-homologously integrating vectors, which can be introduced into the appropriate host cells by any suitable means (transformation, transfection, conjugation, protoplast fusion, electroporation, calcium phosphate-precipitation, direct microinjection, etc.) to transform them.

The nucleic acid constructs can be a plasmid or a stable integration vector for transient or stable expression of IL-15 and/or IL-15Ra and/or an anti-PD-1 antibody molecule in host cells. For stable expression, the vector can mediate chromosomal integration at a target site or a random chromosomal site. Non-limiting examples of host cell-vector systems that may be used to express IL-15 and/or IL-15Ra and/or an anti-PD-1 antibody molecule include mammalian cell systems infected with virus (e.g., vaccinia virus, adenovirus, retroviruses, lentiviruses, etc.); insect cell systems infected with virus (e.g., baculovirus); microorganisms such as yeast containing yeast vectors, or bacteria transformed with bacteriophage, DNA, plasmid DNA, or cosmid DNA; and stable cell lines generated by transformation using a selectable marker. In some embodiments, the nucleic acid constructs include a selectable marker gene including, but not limited to, neomycin (neo), dihydrofolate reductase (dhfr) and hygromycin (hyg).

The nucleic acid constructs can be monocistronic or multicistronic. A multicistronic nucleic acid construct may encode 2, 3, 4, 5, 6, 7, 8, 9, 10 or more, or in the range of 2-5, 5-10 or 10-20 genes/nucleotide sequences. For example, a bicistronic nucleic acid construct may comprise in the following order a promoter, a first gene (e.g., IL-15), and a second gene and (e.g., IL-15Ra). In such a nucleic acid construct, the transcription of both genes is driven by the promoter, whereas the translation of the mRNA from the first gene is by a cap-dependent scanning mechanism and the translation of the mRNA from the second gene is by a cap-independent mechanism, e.g., by an IRES.

Techniques for practicing these aspects will employ, unless otherwise indicated, conventional techniques of molecular biology, microbiology, and recombinant DNA manipulation and production, which are routinely practiced by one of skill in the art. See, e.g., Sambrook, 1989, Molecular Cloning, A Laboratory Manual, Second Edition; DNA Cloning, Volumes I and II (Glover, Ed. 1985); Oligonucleotide Synthesis (Gait, Ed. 1984); Nucleic Acid Hybridization (Hames & Higgins, Eds. 1984); Transcription and Translation (Hames & Higgins, Eds. 1984); Animal Cell Culture (Freshney, Ed. 1986); Immobilized Cells and Enzymes (IRL Press, 1986); Perbal, A Practical Guide to Molecular Cloning (1984); Gene Transfer Vectors for Mammalian Cells (Miller & Calos, Eds. 1987, Cold Spring Harbor Laboratory); Methods in Enzymology, Volumes 154 and 155 (Wu & Grossman, and Wu, Eds., respectively), (Mayer & Walker, Eds., 1987); Immunochemical Methods in Cell and Molecular Biology (Academic Press, London, Scopes, 1987), Expression of Proteins in Mammalian Cells Using Vaccinia Viral Vectors in Current Protocols in Molecular Biology, Volume 2 (Ausubel et al., Eds., 1991).

The host cells chosen for expression of nucleic acids will depend upon the intended use of the cells. Factors such as whether a cell glycosylates similar to cells that endogenously express, e.g., IL-15 and/or IL-15Ra and/or an anti-PD-1 antibody molecule, may be considered in selecting the host cells.

Non-limiting examples of host cells that can be used to express the protein(s) encoded by the nucleic acid constructs herein include mammalian cells, bacterial cells, yeast cells, primary cells, immortalized cells, plant cells and insect cells. In a specific embodiment, the host cells are a mammalian cell line. Examples of mammalian cell lines include, but are not limited to, COS, CHO, HeLa, NIH3T3, HepG2, MCF7, HEK 293, HEK 293T, RD, PC12, hybridomas, pre-B cells, 293, 293H, K562, SkBr3, BT474, A204, MO7Sb, TFI31, Raji, Jurkat, MOLT-4, CTLL-2, MC-IXC, SK-N-MC, SK-N-MC, SK-N-DZ, SH-SYSY, C127, NO, and BE(2)-C cells. Other mammalian cell lines available as hosts for expression are known in the art and include many immortalized cell lines available from the American Type Culture Collection (ATCC).

In a specific embodiment, the nucleic acid constructs encoding IL-15 or IL-15Ra can be co-transfected or transfected into the same host cells or different host cells. Optionally, a nucleic acid construct comprising nucleic acids encoding a selectable marker gene can also be transfected into the same cells to select for transfected cells. If the nucleic acid constructs comprising nucleic acids encoding IL-15 and IL-15Ra are transfected into different cells, IL-15 and IL-15Ra expressed by the different cells can be isolated and contacted with each other under conditions suitable to form IL-15/IL-15Ra complexes described in above. Any techniques known to one of skill in the art can be used to transfect or transducer host cells with nucleic acids including, e.g., transformation, transfection, conjugation, protoplast fusion, electroporation, calcium phosphate-precipitation, direct microinjection, and infection with viruses, including but not limited to adenoviruses, lentiviruses, and retroviruses.

For long-term, high-yield production of recombinant IL-15 and IL-15Ra polypeptides and/or an anti-PD-1 antibody molecule, stable cell lines can be generated. For example, cell lines can be transformed using the nucleic acid constructs described herein which may contain a selectable marker gene on the same or on a separate nucleic acid construct. The selectable marker gene can be introduced into the same cell by co-transfection. Following the introduction of the vector, cells are allowed to grow for 1-2 days in an enriched media before they are switched to selective media to allow growth and recovery of cells that successfully express the introduced nucleic acids. Resistant clones of stably transformed cells may be proliferated using tissue culture techniques well known in the art that are appropriate to the cell type. In a particular embodiment, the cell line has been adapted to grow in serum-free medium. In one embodiment, the cell line has been adapted to grow in serum-free medium in shaker flasks. In one embodiment, the cell line has been adapted to grow in stir or rotating flasks. In certain embodiments, the cell line is cultured in suspension. In particular embodiments, the cell line is not adherent or has been adapted to grow as nonadherent cells. In certain embodiments, the cell line has been adapted to grow in low calcium conditions. In some embodiments, the cell line is cultured or adapted to grow in low serum medium.

In a specific embodiment, a particularly preferred method of high-yield production of a recombinant polypeptide of the present invention is through the use of dihydrofolate reductase (DHFR) amplification in DHFR-deficient CHO cells, by the use of successively increasing levels of methotrexate as described in U.S. Pat. No. 4,889,803. The polypeptide obtained from such cells may be in a glycosylated form.

In one embodiment, cell lines are engineered to express the stable heterodimer of wild-type human IL-15 and wild-type soluble human IL-15Ra, which can then be purified, and administered to a human In one embodiment, the stability of the IL-15/IL-15Ra heterodimer is increased when produced from cell lines recombinantly expressing both IL-15 and IL-15Ra.

In a specific embodiment, the host cell recombinantly expresses IL-15 and the full length IL-15Ra. In another specific embodiment, the host cell recombinantly expresses IL-15 and the soluble form of IL-15Ra. In another specific embodiment, the host cell recombinantly expresses IL-15 and a membrane-bound form of IL-15Ra which is not cleaved from the surface of the cell and remains cell associated. In some embodiments, the host cell recombinantly expressing IL-15 and/or IL-15Ra (full-length or soluble form) also recombinantly expresses another polypeptide (e.g., a cytokine or fragment thereof).

In certain embodiments, such a host cell recombinantly expresses an IL-15 polypeptide in addition to an IL-15Ra polypeptide. The nucleic acids encoding IL-15 and/or IL-15Ra can be used to generate mammalian cells that recombinantly express IL-15 and IL-15Ra in high amounts for the isolation and purification of IL-15 and IL-15Ra, preferably the IL-15 and the IL-15Ra are associated as complexes. In one embodiment, high amounts of IL-15/IL-15Ra complexes refer to amounts of IL-15/IL-15Ra complexes expressed by cells that are at least 1 fold, 2 fold, 3 fold, 4 fold, 5 fold, 6 fold, 7 fold, 8 fold, 9 fold, 10 fold, 20 fold, or more than 20 fold higher than amounts of IL-15/IL-15Ra complexes expressed endogenously by control cells (e.g., cells that have not been genetically engineered to recombinantly express IL-15, IL-15Ra, or both IL-15 and IL-15Ra, or cells comprising an empty vector). In some embodiments, a host cell described herein expresses approximately 0.1 pg to 25 pg, 0.1 pg to 20 pg, 0.1 pg to 15 pg, 0.1 pg to 10 pg, 0.1 pg to 5 pg, 0.1 pg to 2 pg, 2 pg to 10 pg, or 5 to 20 pg of IL-15 as measured by a technique known to one of skill in the art (e.g., an ELISA). In certain embodiments, a host cell described herein expresses approximately 0.1 to 0.25 pg per day, 0.25 to 0.5 pg per day, 0.5 to 1 pg per day, 1 to 2 pg per day, 2 to 5 pg per day, or 5 to 10 pg per day of IL-15 as measured by a technique known to one of skill in the art (e.g., an ELISA). In a specific embodiment, the IL-15Ra is the soluble form of IL-15Ra. In a specific embodiment, the IL-15Ra is the soluble form of IL-15Ra associated with IL-15 in a stable heterodimer, which increases yields and simplifies production and purification of bioactive heterodimer IL-15/soluble IL-15Ra cytokine.

Recombinant IL-15 and IL-15Ra and an anti-PD-1 antibody molecule can be purified using methods of recombinant protein production and purification are well known in the art, e.g., see International Publication No. WO 2007/070488. Briefly, the polypeptide can be produced intracellularly, in the periplasmic space, or directly secreted into the medium. Cell lysate or supernatant comprising the polypeptide can be purified using, for example, hydroxylapatite chromatography, gel electrophoresis, dialysis, and affinity chromatography. Other techniques for protein purification such as fractionation on an ion-exchange column, ethanol precipitation, Reverse Phase HPLC, chromatography on silica, chromatography on heparin SEPHAROSE™ (gel filtration substance; Pharmacia Inc., Piscataway, N.J.) chromatography on an anion or cation exchange resin (such as a polyaspartic acid column), chromatofocusing, SDS-PAGE, and ammonium sulfate precipitation are also available.

In some embodiments, IL-15 and IL-15Ra are synthesized or recombinantly expressed by different cells and subsequently isolated and combined to form an IL-15/IL-15Ra complex, in vitro, prior to administration to a subject. In other embodiments, IL-15 and IL-15Ra are synthesized or recombinantly expressed by different cells and subsequently isolated and simultaneously administered to a subject an IL-15/IL-15Ra complex in situ or in vivo. In yet other embodiments, IL-15 and IL-15Ra are synthesized or expressed together by the same cell, and the IL-15/IL-15Ra complex formed is isolated.

Compositions

Provided herein are compositions comprising an IL-15/IL-15Ra complex. Also provided herein are compositions comprising an anti-PD-1 antibody molecule. The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. The compositions (e.g., pharmaceutical compositions) comprise an effective amount of an IL-15/IL-15Ra complex or anti-PD-1 antibody molecule, or a combination of an IL-15/IL-15Ra complex or anti-PD-1 antibody molecule and a pharmaceutically acceptable carrier. In specific embodiments, the compositions (e.g., pharmaceutical compositions) comprise an effective amount of one or more IL-15/IL-15Ra complexes or an anti-PD-1 antibody molecule and a pharmaceutically acceptable carrier. In some embodiments, the composition further comprises an additional therapeutic, e.g., anti-cancer agent, anti-viral agent, anti-inflammatory agent, adjuvant. Non-limiting examples of such therapeutics are provided infra.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, more preferably, MF59C.1 adjuvant), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. In one embodiment, water is a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Pharmaceutical compositions may be formulated in any conventional manner using one or more pharmaceutically acceptable carriers or excipients. In a specific embodiment, an IL-15/IL-15Ra complex and an anti-PD-1 antibody molecule administered to a subject in accordance with the methods described herein is administered as a pharmaceutical composition.

Generally, the components of the pharmaceutical compositions comprising an IL-15/IL-15Ra complex or an anti-PD-1 antibody molecule are supplied either separately or mixed together in unit dosage form, for example, as a dry lyophilized powder or water free concentrate in a hermetically sealed container such as an ampoule or sachette indicating the quantity of active agent. Where the IL-15/IL-15Ra complex or anti-PD-1 antibody molecule is to be administered by infusion, it can be dispensed with an infusion bottle containing sterile pharmaceutical grade water or saline (e.g., PBS). Where the IL-15/IL-15Ra complex or anti-PD-1 antibody molecule is administered by injection, an ampoule of sterile water for injection or saline can be provided so that the ingredients may be mixed prior to administration.

In some embodiments, the IL-15/IL-15Ra complex or anti-PD-1 antibody molecule may be formulated for administration by any method known to one of skill in the art, including but not limited to, parenteral (e.g., subcutaneous, intravenous, intratumoral or intramuscular) administration. In one embodiment, the IL-15/IL-15Ra complex or anti-PD-1 antibody molecule is formulated for local or systemic parenteral administration, for example intratumoral administration. In a specific embodiment, the IL-15/IL-15Ra complex or anti-PD-1 antibody molecule is formulated for subcutaneous or intravenous administration, respectively. In one embodiment, the IL-15/IL-15Ra complex or anti-PD-1 antibody molecule is formulated in a pharmaceutically compatible solution.

The IL-15/IL-15Ra complex or anti-PD-1 antibody molecule can be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Dose Regimens for Prophylactic and Therapeutic Uses

In one aspect, provided herein are methods for enhancing IL-15-mediated immune function, comprising administering to subjects complexes IL-15/IL-15Ra complexes in a specific dose regimen. Since enhancing IL-15-mediated immune function is beneficial for the prevention, treatment and/or management of certain disorders, provided herein are methods for the prevention, treatment and/or management of such disorders comprising administering to a subject in need thereof IL-15/IL-15Ra complexes. Non-limiting examples of disorders in which it is beneficial to enhance IL-15-mediated immune function include cancer, lymphopenia, immunodeficiencies, infectious diseases, and wounds.

In one embodiment, provided herein is a method for preventing, treating and/or managing disorders in a subject, wherein enhancement of IL-15-mediated immune function is beneficial for the prevention, treatment and/or management of such disorders, the method comprising administering the same dose of an IL-15/IL-15Ra complex to a subject for the duration of the treatment cycle. In one embodiment, the dose is in the range of 0.1 μg/kg and 0.5 μg/kg. In one embodiment, the dose is in the range of 0.25 μg/kg and 1 μg/kg. In a specific embodiment, the dose is in the range of 0.5 μg/kg and 2 μg/kg. In another embodiment, the dose is between 1 μg/kg and 4 μg/kg. In another embodiment, the dose is between 2 μg/kg and 8 μg/kg. In another embodiment, the dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In a specific embodiment, the dose is 1 μg/kg. In certain embodiments, the dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose once per week for a three week period.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Ra complex to the subject in a dosing regimen at least once, twice, four times or six times in a dosing cycle before a period of non-administration. In a specific embodiment the IL-15/IL-15Ra complex is administered once a week for three weeks with no administration in week four. The dosing cycle is then repeated.

In an alternative embodiment, provided herein is a method for preventing, treating and/or managing disorders in a subject, wherein enhancement of IL-15-mediated immune function is beneficial for the prevention, treatment and/or management of such disorders, the method comprising (a) administering at least one initial low dose of an IL-15/IL-15Ra complex to a subject; and (b) administering successively higher doses of the IL-15/IL-15Ra complex to the subject for the duration of the treatment cycle. In a specific embodiment, provided herein is a method for preventing, treating and/or managing cancer in a subject, method comprising (a) administering an initial dose of an IL-15/IL-15Ra complex to the subject for the duration of the treatment cycle; and (b) administering successively higher doses of the IL-15/IL-15Ra complex to the subject for the duration of the treatment cycle. In a specific embodiment, the initial dose is in the range of 0.1 μg/kg and 0.5 μg/kg. In a specific embodiment, the initial dose is in the range of 0.25 μg/kg and 1 μg/kg. In another embodiment, the initial dose is in the range of 0.5 μg/kg and 2μg/kg. In a specific embodiment, the initial dose is between 1 μg/kg and 4 μg/kg. In another embodiment, the initial dose is between 2μg/kg and 8 μg/kg. In another embodiment, the initial dose is about 0.25 μg/kg. In another embodiment, the initial dose is about 0.5 μg/kg. In another embodiment, the initial dose is about 1 μg/kg. In another embodiment, the initial dose is 0.1 μg/kg, 0.25 μg/kg, 0.5 μg/kg, 1 μg/kg, 2 μg/kg, 4 μg/kg, 5 μg/kg, 6 μg/kg, 8 μg/kg. In certain embodiments, the initial dose is administered 1, 2, 3, 4, 5, 6, 7, 8, 9, 10 or more times, or 1 to 3, 1 to 4, 2 to 4, 2 to 5, 2 to 6, 3 to 6, 4 to 6, 6 to 8, 5 to 8, or 5 to 10 times. In some embodiments, the initial dose is administered 1, 2, 3, 4, 5, 6, 7, 8, 9, 10 or more times, or 1 to 3, 1 to 4, 2 to 4, 2 to 5, 1 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each successively higher dose is 1.2, 1.25, 1.3, 1.35, 1.4, 1.45, 1.5, 2, 2.5, 3, 3.5, 4, 4.5, 5, 5.5, or 6 times higher than the previous dose, or 1.2 to 2, 2 to 3, 2 to 4, 1 to 5, 2 to 6, 3 to 4, 3 to 6, or 4 to 6 times higher than the previous dose, or 2 times higher than the previous dose. In some embodiments, each successively higher dose is 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% higher than the previous dose. In specific embodiments, each dose is administered at least 1, 2, 3, 4, 5, 6 or more times over a 5 to 7 day, 5 to 10 day, 7 to 12 day, 7 to 14 day, 7 to 21 day or 14 to 21 day period of time. In another specific embodiment, each dose is administered at least once and the subject is administered a dose three times per 7 day week (e.g., Monday, Wednesday and Friday) for a two week period.

In certain embodiments, the subject is monitored for the following adverse events, such as grade 3 or 4 thrombocytopenia, grade 3 or 4 granulocytopenia, grade 3 or 4 leukocytosis (White Blood Cell (WBC)>100,000 mm³), grade 3 or 4 decreases in WBC, absolute lymphocyte count (ALC) and/or absolute neutrophil count (ANC), lymphocytosis and organ dysfunction (e.g., liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm³), grade 3 or 4 decreases in WBC, absolute lymphocyte count (ALC) and/or absolute neutrophil count (ANC), lymphocytosis, and organ dysfunction (e.g., liver or kidney dysfunction). In accordance with these embodiments, the dose of the IL-15/IL-15Ra complex administered to the subject may be reduced or remain the same until the adverse events decrease or disappear.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Ra complex to the human subject in a dose regimen beginning with a first cycle comprising an initial dose of between 0.25 μg/kg and 4 μg/kg, and sequential cycles wherein the dose is increased two to three times over the previous dose. Each dose is administered at least once, twice, four times or six times before elevating the dose to the next level, and the concentration of free IL-15 in a sample (e.g., a plasma sample) obtained from the subject a certain period of time after the administration of a dose of the IL-15/IL-15Ra complex (e.g., approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Ra complex and before the administration of another dose of the IL-15/IL-15Ra complex) is monitored before elevating the dose to the next level.

In another embodiment, provided herein is a method for preventing, treating and/or managing disorders in a subject, wherein enhancement of IL-15-mediated immune function is beneficial for the prevention, treatment and/or management of such disorders, the method comprising administering an IL-15/IL-15Ra complex to the subject in a dose regimen at the following sequential doses: (i) 0.25 μg/kg; (ii) 0.5 μg/kg; (iii) 1 μg/kg; (iv) 2 μg/kg; (v) 4 μg/kg; and (vi) 8 μg/kg. In a certain embodiment, the IL-15/IL-15Ra complex is administered to the subject in a dose regimen at the following sequential doses: (i) 1 μg/kg; (ii) 2 μg/kg; (iii) 4 μg/kg; and (iv) 8 μg/kg. Each dose is administered at least once, twice, four times or six times in a dosing cycle before elevating the dose to the next level, and wherein the concentration of free IL-15 in a sample (e.g., a plasma sample) obtained from the subject a certain period of time after the administration of a dose of the IL-15/IL-15Ra complex (e.g., approximately 24 hours to approximately 48 hours, approximately 24 hours to approximately 36 hours, approximately 24 hours to approximately 72 hours, approximately 48 hours to approximately 72 hours, approximately 36 hours to approximately 48 hours, or approximately 48 hours to 60 hours after the administration of a dose of the IL-15/IL-15Ra complex and before the administration of another dose of the IL-15/IL-15Ra complex) is monitored before elevating the dose to the next level.

In another embodiment, provided herein is a method for preventing, treating and/or managing cancer in a subject, method comprising administering an IL-15/IL-15Ra complex to the subject in an dose regimen at the following sequential doses: (i) 1 μg/kg; (ii) 2 μg/kg; (iii) 4 μg/kg; and (iv) 8 μg/kg, wherein each dose is administered at least at least once, twice, four times or six times in a dosing cycle before elevating the dose to the next level.

In a particular embodiment, the subject is a human subject. In certain embodiments, the dose in the treatment cycle is administered 1, 2, 3, 4, 5, 6, 7, 8, 9, 10 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times. In some embodiments, the dose is administered 1, 2, 3, 4, 5, 6, 7, 8, 9, 10 or more times, or 1 to 3, 1 to 4, 1 to 5, 2 to 4, 2 to 5, 2 to 6, 1 to 6, 3 to 6, 4 to 6 or 6 to 8 times over a 5 to 7 day, 5 to 10 day, 7 to 12 day, 7 to 14 day, 7 to 21 day or 14 to 21 day period of time. In certain embodiments, each dose is administered 1, 2, 3, 4, 5, 6, 7, 8, 9, 10 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times, per dosing cycle. In specific embodiments, each dose is administered at least 1, 2, 3, 4, 5, 6 or more times, or 1 to 3, 1 to 4, 1 to 5, 2 to 4, 2 to 5, 1 to 6, 2 to 6, 1 to 6, 3 to 6, 4 to 6, 6 to 8, 5 to 8, or 5 to 10 times over a 5 to 7 day, 5 to 10 day, 7 to 12 day, 7 to 14 day, 7 to 21 day or 14 to 21 day period of time.

In another specific embodiment, the subject is administered a dose three times per 7 day week (e.g., Monday, Wednesday and Friday). In certain embodiments, the subject is monitored for the following adverse events, such as grade 3 or 4 thrombocytopenia, grade 3 or 4 granulocytopenia, grade 3 or 4 leukocytosis (White Blood Cell (WBC) >100,000 mm3), grade 3 or 4 decreases in WBC, absolute lymphocyte count (ALC) and/or absolute neutrophil count (ANC), lymphocytosis, and organ dysfunction (e.g., liver or kidney dysfunction). In certain embodiments, the dose is not increased and the dose may be remain the same, be stopped or reduced if the subject experiences adverse events, such as grade 3 or 4 thrombocytopenia, grade 3 or 4 granulocytopenia, grade 3 or leukocytosis (White Blood Cell>100,000 mm3), grade 3 or 4 decreases in WBC, absolute lymphocyte count (ALC) and/or absolute neutrophil count (ANC), lymphocytosis, and organ dysfunction (e.g., liver or kidney dysfunction). In accordance with these embodiments, the dose of the IL-15/IL-15Ra complex administered to the subject may be reduced or remain the same until the adverse events decrease or disappear.

In specific embodiments, in accordance with the methods described herein, each dose is administered once a week for three weeks. In specific embodiments, in accordance with the methods described herein, each dose is administered once, three times a week for two weeks. In specific embodiments, in accordance with the methods described herein, each dose is administered once, three times a week for two, three, or four weeks. In specific embodiments, in accordance with the methods described herein, each dose is administered once, six times a week for two, three, or four weeks. In specific embodiments, in accordance with the methods described herein, each dose is administered once, every other day, for two, three, or four weeks. In specific embodiments, in accordance with the methods described herein, each dose is administered once, every day, for two, three, or four weeks.

In certain embodiments, the IL-15/IL-15Ra complex is administered subcutaneously to a subject in accordance with the methods described herein. In some embodiments, the IL-15/IL-15Ra complex is administered intravenously or intramuscularly to a subject in accordance with the methods described herein. In certain embodiments, the IL-15/IL-15Ra complex is administered intratumorally to a subject in accordance with the methods described herein. In some embodiments, the IL-15/IL-15Ra complex is administered locally to a site (e.g., a site of infection) in a subject in accordance with the methods described herein.

In certain embodiments, a sample obtained from a subject in accordance with the methods described herein is a blood sample. In a specific embodiment, the sample is a plasma sample. Basal plasma levels of IL-15 are approximately 1 pg/ml in humans, approximately 8-10 pg/ml in monkeys (such as macaques), and approximately 12 pg/m in rodents (such as mice). Techniques known to one skilled in the art can be used to obtain a sample from a subject.

In one embodiment, provided herein is a method for preventing, treating and/or managing disorders in a subject, e.g., a hyperproliferative condition or disorder (e.g., a cancer) in a subject including administering to a subject an anti-PD-1 antibody molecule. In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. The dosing schedule (e.g., flat dosing schedule) can vary from e.g., once a week to once every 2, 3, 4, 5, or 6 weeks. In one embodiment, the anti-PD-1 antibody molecule is administered at a dose from about 300 mg to 400 mg once every three weeks or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks.

In accordance with the methods described herein, the IL-15/IL-15Ra complex may be administered to a subject in a pharmaceutical composition. In specific embodiments, the IL-15/IL-15Ra complex is administered in combination with one or more other therapies, e.g., an anti-PD-1 antibody molecule. Combination therapy includes concurrent and successive administration of an IL-15/IL-15Ra complex and an anti-PD-1 antibody molecule. As used herein, the IL-15/IL-15Ra complex and the anti-PD-1 antibody molecule are said to be administered concurrently if they are administered to the patient on the same day, for example, simultaneously, or 1, 2, 3, 4, 5, 6, 7, or 8 hours apart. In contrast, the IL-15/IL-15Ra complex and the anti-PD-1 antibody molecule are said to be administered successively if they are administered to the patient on the different days, for example, the IL-15/IL-15Ra complex and the anti-PD-1 antibody molecule can be administered at a 1-day, 2-day or 3-day interval. In the methods described herein, administration of the IL-15/IL-15Ra complex can precede or follow administration of the anti-PD-1 antibody molecule. When administered simultaneously, the IL-15/IL-15Ra complex and the anti-PD-1 antibody molecule can be in the same pharmaceutical composition or in a different pharmaceutical composition.

In specific embodiments, examples of immune function enhanced by the methods described herein include the proliferation/ expansion of lymphocytes (e.g., increase in the number of lymphocytes), inhibition of apoptosis of lymphocytes, activation of dendritic cells (or antigen presenting cells), and antigen presentation. In particular embodiments, an immune function enhanced by the methods described herein is proliferation/expansion in the number of or activation of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells). In one embodiment, the methods described herein enhance the proliferation/expansion or number of lymphocyte progenitors. In some embodiments, the methods described herein increases the number of CD4⁺ T cells (e.g., Th1 and Th2 helper T cells), CD8⁺ T cells (e.g., cytotoxic T lymphocytes, alpha/beta T cells, and gamma/delta T cells), B cells (e.g., plasma cells), memory T cells, memory B cells, dendritic cells (immature or mature), antigen presenting cells, macrophages, mast cells, natural killer T cells (NKT cells), tumor-resident T cells, CD122⁺ T cells, or natural killer cells (NK cells) by approximately 1 fold, 2 fold, 3 fold, 4 fold, 5 fold, 6 fold, 7 fold, 8 fold, 9 fold, 10 fold, 20 fold, or more relative to a negative control.

In a specific embodiment, the methods described herein enhance or induce immune function in a subject by at least 0.2 fold, 0.5 fold, 0.75 fold, 1 fold, 1.5 fold, 2 fold, 2.5 fold, 3 fold, 4 fold, 5 fold, 6 fold, 7 fold, 8 fold 9 fold, or at least 10 fold relative to the immune function in a subject not administered the combination of an IL-15/IL-15Ra complex and an anti-PD-1 antibody molecule using assays well known in the art, e.g., ELISPOT, ELISA, and cell proliferation assays. In a specific embodiment, the methods described herein enhance or induce immune function in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the immune function in a subject not administered the combination of an IL-15/IL-15Ra complex and an anti-PD-1 antibody molecule using assays well known in the art, e.g., ELISPOT, ELISA, and cell proliferation assays. In a specific embodiment, the immune function is cytokine release (e.g., interferon-gamma, IL-2, IL-5, IL-10, IL-12, or transforming growth factor (TGF) -beta). In one embodiment, the IL-15 mediated immune function is NK cell proliferation, which can be assayed, e.g., by flow cytometry to detect the number of cells expressing markers of NK cells (e.g., CD56). In one embodiment, the IL-15 mediated immune function is CD8⁺ T cell proliferation, which can be assayed, e.g., by flow. In another embodiment, the IL-15 mediated immune function is antibody production, which can be assayed, e.g., by ELISA. In some embodiments, the IL-15 mediated immune function is effector function, which can be assayed, e.g., by a cytotoxicity assay or other assays well known in the art. The effect of one or more doses of a combination of an IL-15/IL-15Ra complex and an anti-PD-1 antibody molecule on peripheral blood lymphocyte counts can be monitored/assessed using standard techniques known to one of skill in the art. Peripheral blood lymphocytes counts in a mammal can be determined by, e.g., obtaining a sample of peripheral blood from said mammal, separating the lymphocytes from other components of peripheral blood such as plasma using, e.g., Ficoll-Hypaque (Pharmacia) gradient centrifugation, and counting the lymphocytes using trypan blue. Peripheral blood T -cell counts in mammal can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS. Further, the effect on a particular subset of T cells (e.g., CD2⁺, CD4⁺, CD8⁺, CD4⁺RO⁺, CD8⁺RO⁺, CD4⁺RA⁺, or CDR⁺RA⁺) or NK cells can be determined using standard techniques known to one of skill in the art such as FACS.

The plasma levels of IL-15 and/or PD-1 can be assessed using standard techniques known to one of skill in the art. For example, plasma can be obtained from a blood sample obtained from a subject and the levels of IL-15 and/or PD-1 in the plasma can be measured by ELISA.

Cancer Treatment

As used herein, the term “cancer” is meant to include all types of cancerous growths or oncogenic processes, metastatic tissues or malignantly transformed cells, tissues, or organs, irrespective of histopathologic type or stage of invasiveness.

Provided herein are methods for preventing, treating, and/or managing cancer, comprising administering an effective amount of an IL-15/IL-15Ra complex and an anti-PD-1 antibody molecule or a composition comprising an IL-15/IL-15Ra complex and an anti-PD-1 antibody molecule to a subject in need thereof. In a specific embodiment, the IL-15/IL-15Ra complex is administered subcutaneously either at the same, repeated dose or alternatively in a dose escalation regimen. In a specific embodiment, anti-PD-1 antibody molecule is administered as an intravenous infusion in a flat dosing regimen.

In specific embodiments, the administration of a combination of an IL-15/IL-15Ra complex and an anti-PD-1 antibody molecule to a subject in accordance with the methods described herein achieves one, two, or three or more results: (1) a reduction in the growth of a tumor or neoplasm; (2) a reduction in the formation of a tumor; (3) an eradication, removal, or control of primary, regional and/or metastatic cancer; (4) a reduction in metastatic spread; (5) a reduction in mortality; (6) an increase in survival rate; (7) an increase in length of survival; (8) an increase in the number of patients in remission; (9) a decrease in hospitalization rate; (10) a decrease in hospitalization lengths; and (11) the maintenance in the size of the tumor so that it does not increase by more than 10%, or by more than 8%, or by more than 6%, or by more than 4%; preferably the size of the tumor does not increase by more than 2%.

In a specific embodiment, the administration of a combination of an IL-15/IL-15Ra complex and an anti-PD-1 antibody molecule to a subject with cancer (in some embodiments, an animal model for cancer) in accordance with the methods described herein inhibits or reduces the growth of a tumor by at least 2 fold, preferably at least 2.5 fold, at least 3 fold, at least 4 fold, at least 5 fold, at least 7 fold, or at least 10 fold relative to the growth of a tumor in a subject with cancer (in some embodiments, in the same animal model for cancer) administered a negative control as measured using assays well known in the art. In another embodiment, the administration of a combination of an IL-15/IL-15Ra complex and an anti-PD-1 antibody molecule to a subject with cancer (in some embodiments, an animal model for cancer) in accordance with the methods described herein inhibits or reduces the growth of a tumor by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to the growth of a tumor in a subject with cancer (in some embodiments, in the same animal model for cancer) administered a negative control, or an IL-15/IL-15Ra complex or an anti-PD-1 antibody molecule as a single agent, as measured using assays well known in the art.

Examples of cancerous disorders include, but are not limited to, solid tumors, hematological cancers, soft tissue tumors, and metastatic lesions. Examples of solid tumors include malignancies, e.g., sarcomas, and carcinomas (including adenocarcinomas and squamous cell carcinomas), of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. Squamous cell carcinomas include malignancies, e.g., in the lung, esophagus, skin, head and neck region, oral cavity, anus, and cervix. In one embodiment, the cancer is a melanoma, e.g., an advanced stage melanoma. Metastatic lesions of the aforementioned cancers can also be treated or prevented using the methods and compositions of the invention.

Exemplary cancers whose growth can be inhibited using the combination an IL-15/IL-15Ra complex and an anti-PD-1 antibody molecule disclosed herein include cancers typically responsive to immunotherapy. Non-limiting examples of preferred cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the combination therapy described herein.

Examples of other cancers that can be treated include bone cancer, pancreatic cancer, skin cancer, cancer of the head or neck, cutaneous or intraocular malignant melanoma, uterine cancer, ovarian cancer, rectal cancer, anal cancer, gastro-esophageal, stomach cancer, testicular cancer, uterine cancer, carcinoma of the fallopian tubes, carcinoma of the endometrium, carcinoma of the cervix, carcinoma of the vagina, carcinoma of the vulva, Merkel cell cancer, Hodgkin lymphoma, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, multiple myeloma, myelodisplastic syndromes, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos (e.g., mesothelioma), and combinations of said cancers.

In a specific embodiment, the cancer is melanoma, renal cancer, colon cancer, or prostate cancer. In another embodiment, the cancer is metastatic.

The combination of an IL-15/IL-15Ra complex and an anti-PD-1 antibody molecule can be administered together with one or more other therapies, e.g., anti-cancer agents, cytokines or anti-hormonal agents, to treat and/or manage cancer. Non-limiting examples anti-cancer agents are described below.

The combination of an IL-15/IL-15Ra complex and an anti-PD-1 antibody molecule can also be administered together with radiation therapy comprising, e.g., the use of x-rays, gamma rays and other sources of radiation to destroy the cancer cells. In specific embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass. An IL-15/IL-15Ra complex and an anti-PD-1 antibody molecule can also be administered in combination with chemotherapy. In one embodiment, an IL-15/IL-15Ra complex and an anti-PD-1 antibody molecule can be administered in accordance with the methods described herein before, during or after radiation therapy or chemotherapy. In one embodiment, a combination of an IL-15/IL-15Ra complex and an anti-PD-1 antibody molecule can be administered before, during or after surgery.

In some embodiments, the combination of an IL-15/IL-15Ra complex and an anti-PD-1 antibody molecule is administered to a subject suffering from or diagnosed with cancer. In other embodiments, the combination of an IL-15/IL-15Ra complex and an anti-PD-1 antibody molecule is administered to a subject predisposed or susceptible to developing cancer.

In certain embodiments, the combination of an IL-15/IL-15Ra complex and an anti-PD-1 antibody molecule is administered to a subject which is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In other embodiments, the combination of an IL-15/IL-15Ra complex and an anti-PD-1 antibody molecule is administered to a human adult. In certain embodiments, the combination of an IL-15/IL-15Ra complex and an anti-PD-1 antibody molecule is administered to a subject that is, will or has undergone surgery, chemotherapy and/or radiation therapy. In some embodiments, the combination of an IL-15/IL-15Ra complex and an anti-PD-1 antibody molecule is administered to refractory patients. In a certain embodiment, refractory patient is a patient refractory to a standard anti-cancer therapy. In certain embodiments, a patient with cancer, is refractory to a therapy when the cancer has not significantly been eradicated and/or the symptoms have not been significantly alleviated. The determination of whether a patient is refractory can be made either in vivo or in vitro by any method known in the art for assaying the effectiveness of a treatment, using art-accepted meanings of “refractory” in such a context. In various embodiments, a patient with cancer is refractory when a cancerous tumor has not decreased or has increased.

Infectious Diseases

Other methods of the invention are used to treat patients that have been exposed to particular toxins or pathogens. Accordingly, another aspect of the invention provides a method of treating an infectious disease in a subject comprising administering to the subject a combination as disclosed herein, e.g., a combination including an IL-15/IL-15Ra complex and an anti-PD-1 antibody molecule, such that the subject is treated for the infectious disease.

In the treatment of infection (e.g., acute and/or chronic), administration of the combination of an IL-15/IL-15Ra complex and an anti-PD-1 antibody molecule can be combined with conventional treatments in addition to or in lieu of stimulating natural host immune defenses to infection. Natural host immune defenses to infection include, but are not limited to inflammation, fever, antibody-mediated host defense, T-lymphocyte-mediated host defenses, including lymphokine secretion and cytotoxic T-cells (especially during viral infection), complement mediated lysis and opsonization (facilitated phagocytosis), and phagocytosis. The ability of the anti-PD-1 antibody molecules to reactivate dysfunctional T-cells would be useful to treat chronic infections, in particular those in which cell-mediated immunity is important for complete recovery.

Antibody mediated PD-1 blockade can act as an adjuvant to IL-15/IL-15Ra complex administration or in combination with an IL-15/IL-15Ra complexes and/or vaccines, to stimulate the immune response to pathogens, toxins and self-antigens. Examples of pathogens for which this therapeutic approach may be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas Aeruginosa Immune system stimulation by IL-15/IL-15Ra complexes and PD-1 blockade is particularly useful against established infections by agents such as HIV that present altered antigens over the course of the infections. These novel epitopes are recognized as foreign at the time of treatment, thus provoking a strong T cell response that is not dampened by negative signals through PD-1, for example.

Additional/Combination Therapy

Other therapies that can be used in combination with an IL-15/IL-15Ra complex and anti-PD-1 antibody molecule, for the prevention, treatment and/or management of a disease, e.g., cancer, infectious disease, lymphopenia, immunodeficiency and wounds, include, but are not limited to, small molecules, synthetic drugs, peptides (including cyclic peptides), polypeptides, proteins, nucleic acids (e.g., DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules. Specific examples of such therapies include, but are not limited to, immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti- inflammatory drugs (e.g., aspirin, ibuprofen, diclofenac, and COX-2 inhibitors), pain relievers, leukotriene antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, ganciclovir, vidarabine, idoxuridine, trifluridine, and ribavirin), foscarnet, amantadine, rimantadine, saquinavir, indinavir, ritonavir, and AZT) and antibiotics (e.g., dactinomycin (formerly actinomycin), bleomycin, erythromycin, penicillin, mithramycin, and anthramycin (AMC)).

Any therapy which is known to be useful, or which has been used or is currently being used for the prevention, management, and/or treatment of a disease that is affected by IL-15 function/signaling and/or immunecheckpoint modulation can be used in combination with a combination therapy of an IL-15II-15Ra complex and anti-PD-1 antibody molecule. See, e.g., Gilman et al., Goodman and Gilman's, The Pharmacological Basis of Therapeutics, 10th ed., McGraw-Hill, New York, 2001; The Merck Manual of Diagnosis and Therapy, Berkow, M.D. et al. (eds.), 17th Ed., Merck Sharp & Dohme Research Laboratories, Rahway, N.J., 1999; Cecil Textbook of Medicine, 20th Ed., Bennett and Plum (eds.), W.B. Saunders, Philadelphia, 1996, and Physicians' Desk Reference (66th ed. 2012) for information regarding therapies (e.g., prophylactic or therapeutic agents) which have been or are currently being used for preventing, treating and/or managing disease or disorder, e.g., cancer, infectious disease, lymphopenia, immunodeficiency and wounds.

Non-limiting examples of one or more other therapies that can be used in addition to a combination therapy of an IL-15/II-15Ra complex and anti-PD-1 antibody molecule include immunomodulatory agents, such as but not limited to, chemotherapeutic agents and non-chemotherapeutic immunomodulatory agents. Non-limiting examples of chemotherapeutic agents include methotrexate, cyclosporin A, leflunomide, cisplatin, ifosfamide, taxanes such as taxol and paclitaxol, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin homologs, and cytoxan.

Biological Activity

In one aspect, the IL-15/IL-15Ra complex and/or anti-PD-1 antibody molecule increases an immune response that can be, e.g., an antibody response (humoral response) or a cellular immune response, e.g., cytokine secretion (e.g., interferon-gamma), helper activity or cellular cytotoxicity. In one embodiment, the increased immune response is increased cytokine secretion, antibody production, effector function, T cell proliferation, and/or NK cell proliferation. Various assays to measure such activities are well known in the art, and include enzyme-linked immunosorbent assays (ELISA; see e.g., in Section 2.1 of Current Protocols in Immunology, Coligan et al. (eds.), John Wiley and Sons, Inc. 1997), a “tetramer staining” assay to identify antigen-specific T-cells (see Altman et al., (1996), Science 274: 94-96), a mixed lymphocyte target culture assay (see e.g., in Palladino et al., (1987), Cancer Res. 47:5074-5079) and an ELISPOT assay that can be used to measure cytokine release in vitro (see, e.g., Scheibenbogen et al., (1997), Int. J. Cancer 71:932-936).

In some aspects, the immune response induced or enhanced by a combination of an IL-15/IL-15Ra complex and an anti-PD-1 antibody molecule is enhanced or increased by at least 2 fold, 3 fold, 4 fold, 5 fold, 6 fold, 7 fold, 8 fold, 9 fold, 10 fold, 11 fold, or 12 fold relative to an immune response elicited by a negative control, or by an IL-15/IL-15Ra complexes or an anti-PD-1 antibody molecule administered as a single agent, as assayed by any known method in the art. In certain embodiments, the immune response induced by the combination of an IL-15/IL-15Ra complex and an anti-PD-1 antibody molecule is enhanced by at least 0.5-2 times, at least 2-5 times, at least 5-10 times, at least 10-50 times, at least 50-100 times, at least 100-200 times, at least 200-300 times, at least 300-400 times or at least 400-500 times relative to the immune response induced by a negative control as assayed by any known method in the art. In specific embodiments, the assay used to assess immune response measures the level of antibody production, cytokine production, or cellular cytotoxicity, and such assays are well known in the art. In some embodiments, the assay used to measure the immune response is an enzyme-linked immunosorbent assay (ELISA) that determines antibody or cytokine levels, an ELISPOT assay that determines cytokine release, or a ['Cr] release assay that determines cellular cytotoxicity.

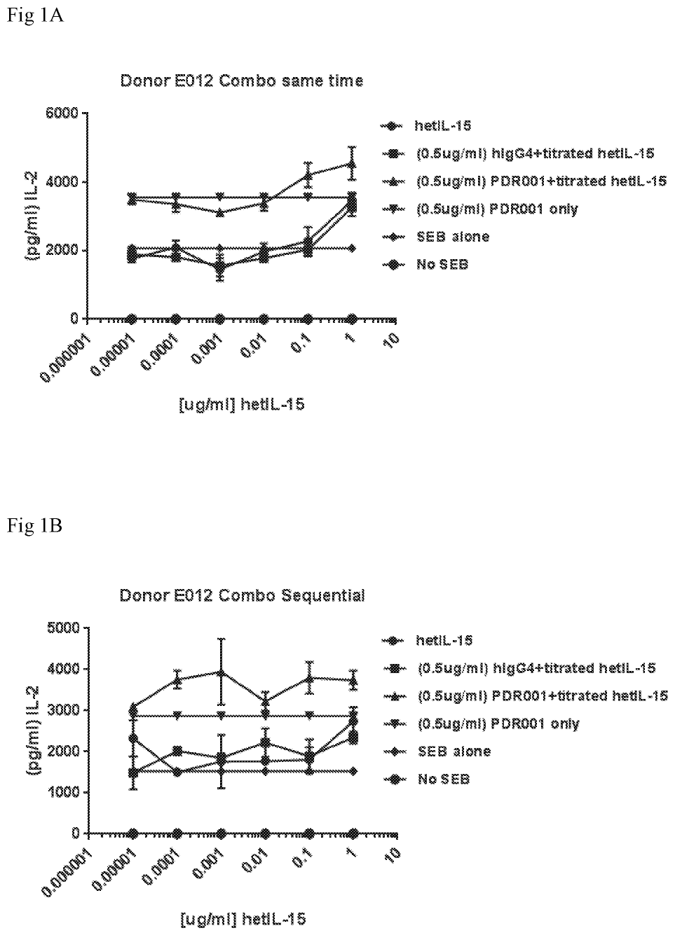

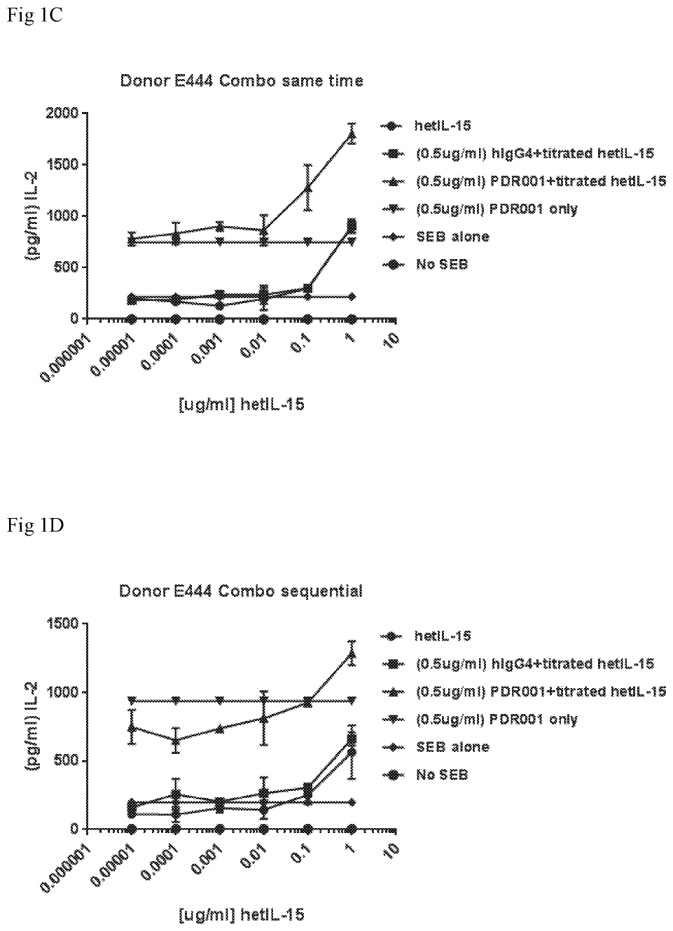

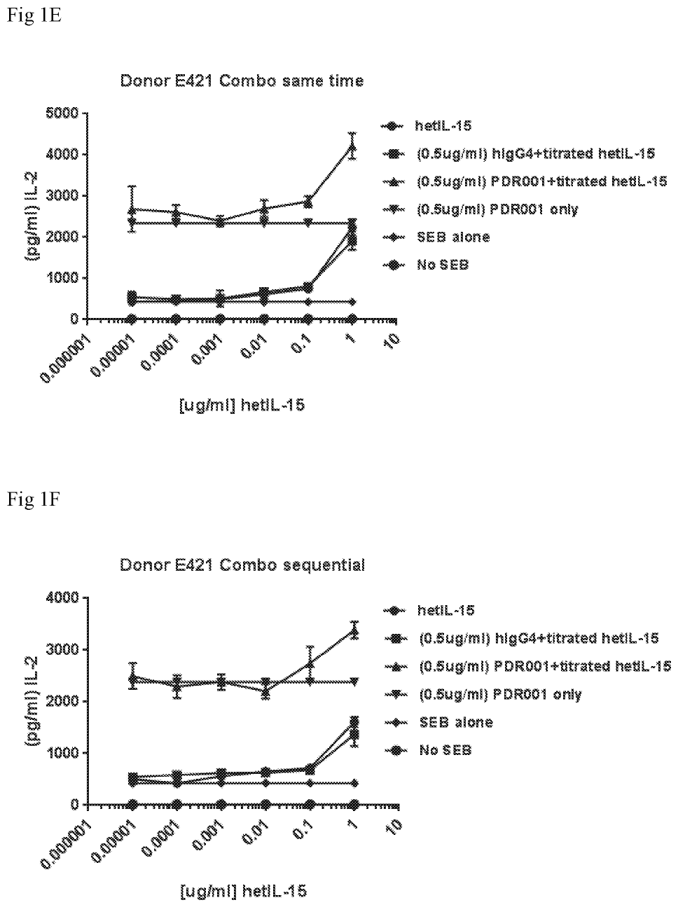

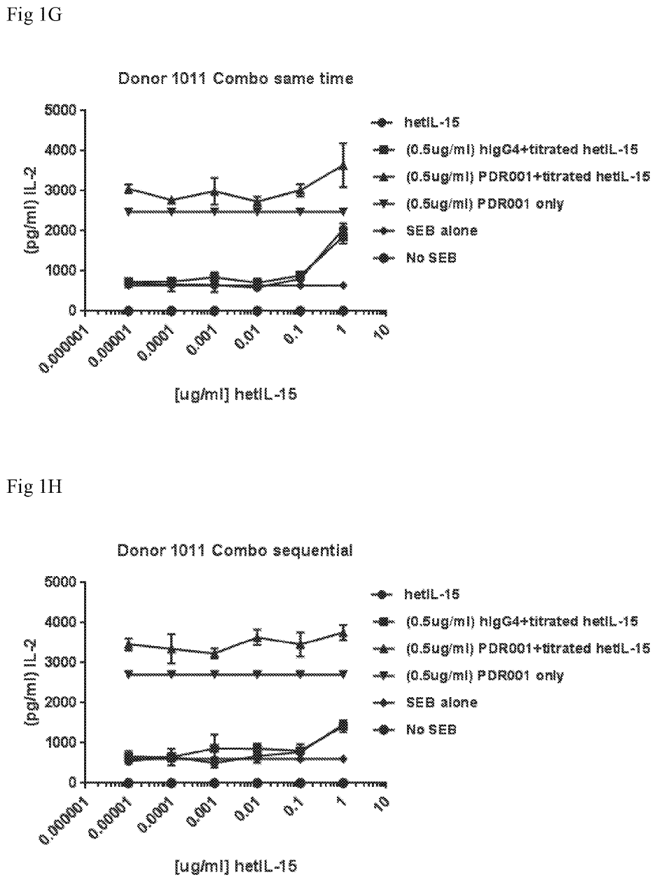

In a specific embodiment, the combination of an IL-15/IL-15Ra complex and an anti-PD-1 antibody molecule increases the expression of IL-2 on whole blood activated by Staphylococcal enterotoxin B (SEB). For example, the IL-15/IL-15Ra complex and an anti-PD-1 antibody molecule increases the expression of IL-2 by at least about 2, 3, 4, or 5-fold, compared to the expression of IL-2 when an the IL-15/IL-15Ra complex, the anti-PD-1 antibody molecule or an isotype control (e.g., IgG4) is used alone. Such an effect is demonstrated in Example 1 and shown in FIGS. 1 to 4. The additive or synergistic effect was more pronounced when the IL-15/IL-15Ra complex was administered on the same day as the anti-PD-1 antibody, rather than when the IL-15/IL-15Ra complex was administered 72 hours after administration of the anti-PD-1 antibody molecule.

In one embodiment, the proliferation or viability of cancer cells contacted with a combination of an IL-15/IL-15Ra complex and an anti-PD-1 antibody molecule is inhibited or reduced by at least 2 fold, preferably at least 2.5 fold, at least 3 fold, at least 4 fold, at least 5 fold, at least 7 fold, or at least 10 fold relative to the proliferation of the cancer cells when contacted with a negative control or an IL-15/IL-15Ra complex or an anti-PD-1 antibody molecule as a single agent, as measured using assays well known in the art, e.g., cell proliferation assays using CSFE, BrdU, and radioactive thymidine incorporation. Alternatively, cell viability can be measured by assays that measure lactate dehydrogenase (LDH), a stable cytosolic enzyme that is released upon cell lysis, or by the release of [⁵¹Cr] upon cell lysis. In another embodiment, the proliferation of cancer cells contacted with a combination of an IL-15/IL-15Ra complex and an anti-PD-1 antibody molecule is inhibited or reduced by at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% relative to cancer cells contacted with a negative control or an IL-15/IL-15Ra complex or an anti-PD-1 antibody molecule as a single agent, as measured using assays well known in the art, e.g., cell proliferation assays using CSFE, BrdU, and radioactive thymidine incorporation.

Cancer cell lines on which such assays can be performed are well known to those of skill in the art. Necrosis, apoptosis and proliferation assays can also be performed on primary cells, e.g., a tissue explant.

In one embodiment, necrotic cells are measured by the ability or inability of the cell to take up a dye such as neutral red, trypan blue, or ALAMARTM blue (Page et al., (1993), Intl. J. of Oncology 3:473-476). In such an assay, the cells are incubated in media containing the dye, the cells are washed, and the remaining dye, reflecting cellular uptake of the dye, is measured spectrophotometrically. In another embodiment, the dye is sulforhodamine B (SRB), whose binding to proteins can be used as a measure of cytotoxicity (Skehan et al., (1990), J. Natl Cancer Inst. 82:1107-12). In yet another embodiment, a tetrazolium salt, such as MTT, is used in a quantitative colorimetric assay for mammalian cell survival and proliferation by detecting living, but not dead, cells (see, e.g., Mosmann, (1983), J. Immunol. Methods 65:55-63).

In other embodiments, apoptotic cells are measured in both the attached and “floating” compartments of the cultures. Both compartments are collected by removing the supernatant, trypsinizing the attached cells, and combining both preparations following a centrifugation wash step (10 minutes, 2000 rpm). The protocol for treating tumor cell cultures with sulindac and related compounds to obtain a significant amount of apoptosis has been described in the literature (see, e.g., Piazza et al., (1995) Cancer Research 55:3110-16). Features of this method include collecting both floating and attached cells, identification of the optimal treatment times and dose range for observing apoptosis, and identification of optimal cell culture conditions. In another embodiment, apoptosis is quantitated by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, (1999), no. 2, pp. 34-37 (Roche Molecular Biochemicals). In yet another embodiment, apoptosis can be observed morphologically.

### Specific Embodiments, Citation and References

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various references, including patent applications, patents, and scientific publications, are cited herein; the disclosure of each such reference is hereby incorporated herein by reference in its entirety.

TABLE 1
Sequence Table

| | | |
|---|---|---|
| SEQ | | |
| ID | | |
| NO | Description | Sequence |

IL-15 related sequences

| | | |
|---|---|---|
| 1 | Human IL-15 | MRISKPHLRSISIQCYLCULNSHFLTEAGIHVFILGCFSAGLPKTEANW |
| | (with signal | VNVISDLKKIEDLIQSMHIDATLYTESDVHPSCKVTAMKCFLLELQVISLES |
| | peptide) | GDASIHDTVENLIILANNSLSSNGNVTESGCKECEELEEKNIKEFLQSFVHI |
| | | VQMFINTS |
| 2 | Human IL-15 | atgagaattt cgaaaccaca tttgagaagt atttccatcc agtgctactt gtgtttactt ctaaacagtc |
| | DNA (with signal | attttctaac tgaagctggc attcatgtct tcattttggg ctgtttcagt gcagggcttc ctaaaacaga |
| | peptide) | agccaactgg gtgaatgtaa taagtgattt gaaaaaaatt gaagatctta ttcaatctat gcatattgat |
| | | gctactttat atacggaaag tgatgttcac cccagttgca aagtaacagc aatgaagtgc tttctcttgg |
| | | agttacaagt tatttcactt gagtccggag atgcaagtat tcatgataca gtagaaaatc tgatcatcct |
| | | agcaaacaac agtttgtctt ctaatgggaa tgtaacagaa tctggatgca aagaatgtga |
| | | ggaactggag gaaaaaaata ttaaagaatt tttgcagagt tttgtacata ttgtccaaat gttcatcaac |
| | | acacttga |
| 3 | human IL-15 | atgtggctcc agagcctgct actcctgggg acggtggcct gcagcatctc gaactgggtg aacgtgatct |
| | with GMCSF | cggacctgaa gaagatcgag gacctcatcc agtcgatgca catcgacgcg acgctgtaca |
| | signal peptide | cggagtcgga cgtccacccg tcgtgcaagg tcacggcgat gaagtgcttc ctcctggagc tccaagtcat |
| | | ctcgctcgag tcgggggacg cgtcgatcca cgacacggtg gagaacctga tcatcctggc |
| | | gaacaactcg ctgtcgtcga acgggaacgt cacggagtcg ggctgcaagg agtgcgagga |
| | | gctggaggag aagaacatca aggagttcct gcagtcgttc gtgcacatcg tccagatgtt catcaacacg |
| | | tcgtga |
| 4 | IL-15 codon | cctggccatt gcatacgttg tatccatatc ataatatgta catttatatt ggctcatgtc caacattacc |
| | optimized DNA | gccatgttga cattgattat tgactagtta ttaatagtaa tcaattacgg ggtcattagt tcatagccca |
| | | tatatggagt tccgcgttac ataacttacg gtaaatggcc cgcctggctg accgcccaac gacccccgcc |
| | | cattgacgtc aataatgacg tatgttccca tagtaacgcc aatagggact ttccattgac gtcaatgggt |
| | | ggagtattta cggtaaactg cccacttggc agtacatcaa gtgtatcata tgccaagtac gccccctatt |
| | | gacgtcaatg atggtaaatg gcccgcctgg cattatgccc agtacatgac cttatgggac tttcctactt |
| | | ggcagtacat ctacgtatta gtcatcgcta ttaccatggt gatgcggttt tggcagtaca tcaatgggcg |
| | | tggatagcgg tttgactcac ggggatttcc aagtctccac cccattgacg tcaatgggag tttgttttgg |
| | | caccaaaatc aacgggactt tccaaaatgt cgtaacaact ccgccccatt gacgcaaatg ggcggtaggc |
| | | gtgtacggtg ggaggtctat ataagcagag ctcgtttagt gaaccgtcag atcgcctgga gacgccatcc |
| | | acgctgtttt gacctccata gaagacaccg ggaccgatcc agcctccgcg ggcgcgcgtc |
| | | gacaagaaat gcggatctcg aagccgcacc tgcggtcgat atcgatccag tgctacctgt gcctgctcct |
| | | gaactcgcac ttcctcacgg aggccggtat acacgtcttc atcctgggct gcttctcggc ggggctgccg |
| | | aagacggagg cgaactgggt gaacgtgatc tcggacctga agaagatcga ggacctcatc |
| | | cagtcgatgc acatcgacgc gacgctgtac acggagtcgg acgtccaccc gtcgtgcaag |
| | | gtcacggcga tgaagtgctt cctcctggag ctccaagtca tctcgctcga gtcgggggac gcgtcgatcc |
| | | acgacacggt ggagaacctg atcatcctgg cgaacaactc gctgtcgtcg aacgggaacg |
| | | tcacggagtc gggctgcaag gagtgcgagg agctggagga gaagaacatc aaggagttcc |
| | | tgcagtcgtt cgtgcacatc gtccagatgt tcatcaacac gtcgtgaggg cccggcgcgc cgaattcgcg |
| | | gatatcggtt aacggatcca gatctgctgt gccttctagt tgccagccat ctgttgtttg cccctccccc |
| | | gtgccttcct tgaccctgga aggtgccact cccactgtcc tttcctaata aaatgaggaa attgcatcgc |
| | | attgtctgag taggtgtcat tctattctgg ggggtggggt ggggcaggac agcaaggggg |
| | | aggattggga agacaatagc aggcatgctg gggatgcggt gggctctatg ggtacccagg |
| | | tgctgaagaa ttgacccggt tcctcctggg ccagaaagaa gcaggcacat ccccttctct gtgacacacc |
| | | ctgtccacgc ccctggttct tagttccagc cccactcata ggacactcat agctcaggag ggctccgcct |
| | | tcaatcccac ccgctaaagt acttggagcg gtctctccct ccctcatcag cccaccaaac caaacctagc |
| | | ctccaagagt gggaagaaat taaagcaaga taggctatta agtgcagagg gagagaaaat |
| | | gcctccaaca tgtgaggaag taatgagaga aatcata |
| 5 | IL-15 codon | MRISKPHLRSISIQCYLCLLLNSHFLTEAGIHVFILGCFSAGLPKTEANW |
| | optimized amino | VNVISDLKKIEDLIQSMHIDATLYTESDVHPSCKVTAMKCFLLELQVIS |
| | acid | LESGDASIHDTVENLIILANNSLSSNGNVTESGCKECEELEEKNIKEFLQ |
| | | SFVHIVQMFINTS |
| 6 | Human IL-15Ra | MAPRRARGCRTLGLPALLLLLLLRPPATRGITCPPPMSVEHADIWVKSY |
| | with signal | SLYSRERYICNSGFKRKAGTSSLTECVLNKATNVAHWTTPSLKCIRDPALVH |
| | peptide | QRPAPPSTVTTAGVTPQPESLSPSGKEPAASSPSSNNTAATTAAIVPGSQLM |
| | | PSKSPSTGTTEISSHESSHGTPSQTTAKNWELTASASHQPPGVYPQGHSDTT |
| | | VAISTSTVLLCGLSAVSLLACYLKSRQTPPLASVEMEAMEALPVTWGTSSRD |
| | | EDLENCSHHL |
| 7 | Human soluble | MAPRRARGCRTLGLPALLLLLLLRPPATRGITCPPPMSVEHADIWVKSY |
| | IL-15Ra with | SLYSRERYICNSGFKRKAGTSSLTECVLNKATNVAHWTTPSLKCIRDPALVH |
| | signal peptide | QRPAPPSTVTTAGVTPQPESLSPSGKEPAASSPSSNNTAATTAAIVPGSQLM |
| | | PSKSPSTGTTEISSHESSHGTPSQTTAKNWELTASASHQPPGVYPQG |
| 8 | Human IL-15Ra | atggccccgc ggcgggcgcg cggctgccgg accctcggtc tcccggcgct gctactgctg |
| | with signal | ctgctgctcc ggccgccggc gacgcggggc atcacgtgcc ctccccccat gtccgtggaa |
| | peptide DNA | cacgcagaca tctgggtcaa gagctacagc ttgtactcca gggagcggta catttgtaac |
| | | tctggtttca agcgtaaagc cggcacgtcc agcctgacgg agtgcgtgtt gaacaaggcc |
| | | acgaatgtcg cccactggac aacccccagt ctcaaatgca ttagagaccc tgccctggtt |
| | | caccaaaggc cagcgccacc ctccacagta acgacggcag gggtgacccc acagccagag |
| | | agcctctccc cttctggaaa agagcccgca gcttcatctc ccagctcaaa caacacagcg |
| | | gccacaacag cagctattgt cccgggctcc cagctgatgc cttcaaaatc accttccaca |
| | | ggaaccacag agataagcag tcatgagtcc tcccacggca ccccctctca gacaacagcc |
| | | aagaactggg aactcacagc atccgcctcc caccagccgc caggtgtgta tccacagggc |
| | | cacagcgaca ccactgtggc tatctccacg tccactgtcc tgctgtgtgg gctgagcgct gtgtctctcc |
| | | tggcatgcta cctcaagtca aggcaaactc ccccgctggc cagcgttgaa atggaagcca |
| | | tggaggctct gccggtgact tgggggacca gcagcagaga tgaagacttg gaaaactgct |
| | | ctcaccacct atga |
| 9 | Human soluble | atggccccgc ggcgggcgcg cggctgccgg accctcggtc tcccggcgct gctactgctg |
| | IL-15Ra with | ctgctgctcc ggccgccggc gacgcggggc atcacgtgcc ctccccccat gtccgtggaa |
| | signal peptide | cacgcagaca tctgggtcaa gagctacagc ttgtactcca gggagcggta catttgtaac |
| | DNA | tctggtttca agcgtaaagc cggcacgtcc agcctgacgg agtgcgtgtt gaacaaggcc |
| | | acgaatgtcg cccactggac aacccccagt ctcaaatgca ttagagaccc tgccctggtt |
| | | caccaaaggc cagcgccacc ctccacagta acgacggcag gggtgacccc acagccagag |
| | | agcctctccc cttctggaaa agagcccgca gcttcatctc ccagctcaaa caacacagcg |
| | | gccacaacag cagctattgt cccgggctcc cagctgatgc cttcaaaatc accttccaca |
| | | ggaaccacag agataagcag tcatgagtcc tcccacggca ccccctctca gacaacagcc |
| | | aagaactggg aactcacagc atccgcctcc caccagccgc caggtgtgta tccacagggc |
| 10 | Human soluble | ITCPPPMSVEHADIWVKSYSLYSRERYICNSGFKRKAGTSSLTECVLN |
| | IL-15Ra (PQG | KATNVAHWTTPSLKCIRDPALVHQRPAPPSTVTTAGVTPQPESLSPSG |
| | termination | KEPAASSPSSNNTAATTAAIVPGSQLMPSKSPSTGTTEISSHESSHGTPS |
| | (“PQG” | QTTAKNWELTASASHQPPGVYPQG |
| | disclosed as | |
| | SEQ ID NO: 20)) | |
| 11 | IL-15Ra codon | cctggccatt gcatacgttg tatccatatc ataatatgta catttatatt ggctcatgtc caacattacc |
| | optimized DNA | gccatgttga cattgattat tgactagtta ttaatagtaa tcaattacgg ggtcattagt tcatagccca |
| | | tatatggagt tccgcgttac ataacttacg gtaaatggcc cgcctggctg accgcccaac gacccccgcc |
| | | cattgacgtc aataatgacg tatgttccca tagtaacgcc aatagggact ttccattgac gtcaatgggt |
| | | ggagtattta cggtaaactg cccacttggc agtacatcaa gtgtatcata tgccaagtac gccccctatt |
| | | gacgtcaatg atggtaaatg gcccgcctgg cattatgccc agtacatgac cttatgggac tttcctactt |
| | | ggcagtacat ctacgtatta gtcatcgcta ttaccatggt gatgcggttt tggcagtaca tcaatgggcg |
| | | tggatagcgg tttgactcac ggggatttcc aagtctccac cccattgacg tcaatgggag tttgttttgg |
| | | caccaaaatc aacgggactt tccaaaatgt cgtaacaact ccgccccatt gacgcaaatg ggcggtaggc |
| | | gtgtacggtg ggaggtctat ataagcagag ctcgtttagt gaaccgtcag atcgcctgga gacgccatcc |
| | | acgctgtttt gacctccata gaagacaccg ggaccgatcc agcctccgcg ggcgcgcgtc |
| | | gacgctagca agaaatggcc ccgaggcggg cgcgaggctg ccggaccctc ggtctcccgg |
| | | cgctgctact gctcctgctg ctccggccgc cggcgacgcg gggcatcacg tgcccgcccc |
| | | ccatgtccgt ggagcacgca gacatctggg tcaagagcta cagcttgtac tcccgggagc ggtacatctg |
| | | caactcgggt ttcaagcgga aggccggcac gtccagcctg acggagtgcg tgttgaacaa |
| | | ggccacgaat gtcgcccact ggacgacccc ctcgctcaag tgcatccgcg acccggccct |
| | | ggttcaccag cggcccgcgc caccctccac cgtaacgacg gcgggggtga ccccgcagcc |
| | | ggagagcctc tccccgtcgg gaaaggagcc cgccgcgtcg tcgcccagct cgaacaacac |
| | | ggcggccaca actgcagcga tcgtcccggg ctcccagctg atgccgtcga agtcgccgtc |
| | | cacgggaacc acggagatca gcagtcatga gtcctcccac ggcaccccct cgcaaacgac |
| | | ggccaagaac tgggaactca cggcgtccgc ctcccaccag ccgccggggg tgtatccgca |
| | | aggccacagc gacaccacgg tggcgatctc cacgtccacg gtcctgctgt gtgggctgag |
| | | cgcggtgtcg ctcctggcgt gctacctcaa gtcgaggcag actcccccgc tggccagcgt |
| | | tgagatggag gccatggagg ctctgccggt gacgtggggg accagcagca gggatgagga |
| | | cttggagaac tgctcgcacc acctataatg agaattcgat ccagatctgc tgtgccttct agttgccagc |
| | | catctgttgt ttgcccctcc cccgtgcctt ccttgaccct ggaaggtgcc actcccactg tcctttccta |
| | | ataaaatgag gaaattgcat cgcattgtct gagtaggtgt cattctattc tggggggtgg ggtggggcag |
| | | gacagcaagg gggaggattg ggaagacaat agcaggcatg ctggggatgc ggtgggctct |
| | | atgggtaccc aggtgctgaa gaattgaccc ggttcctcct gggccagaaa gaagcaggca |
| | | catccccttc tctgtgacac accctgtcca cgcccctggt tcttagttcc agccccactc ataggacact |
| | | catagctcag gagggctccg ccttcaatcc cacccgctaa agtacttgga gcggtctctc cctccctcat |
| | | cagcccacca aaccaaacct agcctccaag agtgggaaga aattaaagca agataggcta |
| | | ttaagtgcag agggagagaa aatgcctcca acatgtgagg aagtaatgag agaaatcata |
| 12 | IL-15Ra codon | MAPRRARGCRTLGLPALLLLLLLRPPATRGITCPPPMSVEHADIWVKS |
| | optimized amino | YSLYSRERYICNSGFKRKAGTSSLTECVLNKATNVAHWTTPSLKCIRD |
| | acid | PALVHQRPAPPSTVTTAGVTPQPESLSPSGKEPAASSPSSNNTAATTAA |
| | | IVPGSQLMPSKSPSTGTTEISSHESSHGTPSQTTAKNWELTASASHQPP |
| | | GVYPQGHSDTTVAISTSTVLLCGLSAVSLLACYLKSRQTPPLASVEME |
| | | AMEALPVTWGTSSRDEDLENCSHHL |
| 13 | CMV IL-15Ra | cctggccatt gcatacgttg tatccatatc ataatatgta catttatatt ggctcatgtc caacattacc |
| | codon optimized | gccatgttga cattgattat tgactagtta ttaatagtaa tcaattacgg ggtcattagt tcatagccca |
| | DNA | tatatggagt tccgcgttac ataacttacg gtaaatggcc cgcctggctg accgcccaac gacccccgcc |
| | | cattgacgtc aataatgacg tatgttccca tagtaacgcc aatagggact ttccattgac gtcaatgggt |
| | | ggagtattta cggtaaactg cccacttggc agtacatcaa gtgtatcata tgccaagtac gccccctatt |
| | | gacgtcaatg atggtaaatg gcccgcctgg cattatgccc agtacatgac cttatgggac tttcctactt |
| | | ggcagtacat ctacgtatta gtcatcgcta ttaccatggt gatgcggttt tggcagtaca tcaatgggcg |
| | | tggatagcgg tttgactcac ggggatttcc aagtctccac cccattgacg tcaatgggag tttgttttgg |
| | | caccaaaatc aacgggactt tccaaaatgt cgtaacaact ccgccccatt gacgcaaatg ggcggtaggc |
| | | gtgtacggtg ggaggtctat ataagcagag ctcgtttagt gaaccgtcag atcgcctgga gacgccatcc |
| | | acgctgtttt gacctccata gaagacaccg ggaccgatcc agcctccgcg ggcgcgcgtc |
| | | gacgctagca agaaatggcc ccgaggcggg cgcgaggctg ccggaccctc ggtctcccgg |
| | | cgctgctact gctcctgctg ctccggccgc cggcgacgcg gggcatcacg tgcccgcccc |
| | | ccatgtccgt ggagcacgca gacatctggg tcaagagcta cagcttgtac tcccgggagc ggtacatctg |
| | | caactcgggt ttcaagcgga aggccggcac gtccagcctg acggagtgcg tgttgaacaa |
| | | ggccacgaat gtcgcccact ggacgacccc ctcgctcaag tgcatccgcg acccggccct |
| | | ggttcaccag cggcccgcgc caccctccac cgtaacgacg gcgggggtga ccccgcagcc |
| | | ggagagcctc tccccgtcgg gaaaggagcc cgccgcgtcg tcgcccagct cgaacaacac |
| | | ggcggccaca actgcagcga tcgtcccggg ctcccagctg atgccgtcga agtcgccgtc |
| | | cacgggaacc acggagatca gcagtcatga gtcctcccac ggcaccccct cgcaaacgac |
| | | ggccaagaac tgggaactca cggcgtccgc ctcccaccag ccgccggggg tgtatccgca |
| | | aggccacagc gacaccacgt aatgagaatt cgcggatatc ggttaacgga tccagatctg ctgtgccttc |
| | | tagttgccag ccatctgttg tttgcccctc ccccgtgcct tccttgaccc tggaaggtgc cactcccact |
| | | gtcctacct aataaaatga ggaaattgca tcgcattgtc tgagtaggtg tcattctatt ctggggggtg |
| | | gggtggggca ggacagcaag ggggaggatt gggaagacaa tagcaggcat gctggggatg |
| | | cggtgggctc tatgggtacc caggtgctga agaattgacc cggttcctcc tgggccagaa |
| | | agaagcaggc acatcccctt ctctgtgaca caccctgtcc acgcccctgg acttagttc cagccccact |
| | | cataggacac tcatagctca ggagggctcc gccttcaatc ccacccgcta aagtacttgg agcggtctct |
| | | ccctccctca tcagcccacc aaaccaaacc tagcctccaa gagtgggaag aaattaaagc |
| | | aagataggct attaagtgca gagggagaga aaatgcctcc aacatgtgag gaagtaatga |
| | | gagaaatcat a |
| 14 | CMV IL-15Ra | MAPRRARGCRTLGLPALLLLLLLRPPATRGITCPPPMSVEHADIWVKS |
| | codon optimized | YSLYSRERYICNSGFKRKAGTSSLTECVLNKATNVAHWTTPSLKCIRD |
| | amino acid | PALVHQRPAPPSTVTTAGVTPQPESLSPSGKEPAASSPSSNNTAATTAA |
| | | IVPGSQLMPSKSPSTGTTEISSHESSHGTPSQTTAKNWELTASASHQPP |
| | | GVYPQGHSDTT |
| 15 | C-terminal of | PQGHSDTT |
| | soluble human | |
| | IL-15Ra | |
| 16 | C-terminal of | PQGHSDT |
| | soluble human | |
| | IL-15Ra | |
| 17 | C-terminal of | PQGHSD |
| | soluble human | |
| | IL-15Ra | |
| 18 | C-terminal of | PQGHS |
| | soluble human | |
| | IL-15Ra | |
| 19 | C-terminal of | PQGH |
| | soluble human | |
| | IL-15Ra | |
| 20 | C-terminal of | PQG |
| | soluble human | |
| | IL-15Ra | |
| 21 | Human soluble | ITCPPPMSVEHADIWVKSYSLYSRERYICNSGFKRKAGTSSLTECVLN |
| | IL-15Ra | KATNVAHWTTPSLKCIRDPALVHQRPAPPSTVTTAGVTPQPESLSPSG |
| | | KEPAASSPSSNNTAATTAAIVPGSQLMPSKSPSTGTTEISSHESSHGTPS |
| | | QTTAKNWELTASASHQPPGVYPQGHSDTT |
| 22 | IL-15Ra O- | NWELTASASHQPPGVYPQG |
| | glycosylation | |
| 23 | IL-15Ra N- | ITCPPPMSVEHADIWVK |
| | glycosylation | |
| 24 | IL-15Ra N- | ITCPPPMSVEHADIWVKSYSLYSRERYICNS |
| | glycosylation | |
| 25 | IL-15Ra | RXXR |
| | heterologous | |
| | protease cleavage | |
| | site recognized | |
| | by furin protease | |
| | Xaa = any amino | |
| | acid | |
| 26 | IL-15Ra | XXPRXX |
| | heterologous | |
| | protease cleavage | |
| | site | |
| | 1,2 Xaa = | |
| | hydrophobic | |
| | amino acids | |
| | 5,6 Xaa = non- | |
| | acidic amino | |
| | acids | |
| 27 | synthetic sIL- | MAPRRARGCRTLGLPALLLLLLLRPPATRGITCPPPMSVEHADIWVKS |
| | 15Ralpha-Fc | YSLYSRERYICNSGFKRKAGTSSLTECVLNKATNVAHWTTPSLKCIRD |
| | fusion protein | PALVHQRPAPPSTVTTAGVTPQPESLSPSGKEPAASSPSSNNTAATTAA |
| | huIL15sRa205- | IVPGSQLMPSKSPSTGTTEISSHESSHGTPSQTTAKNWELTASASHQPP |
| | Fc | GVYPQGHSDTTPKSCDKTHTCPPCPAPELLGGPSVFLFPPKPKDTLMIS |
| | | RTPEVTCVVVDVSHEDPEVKFNWYVDGVEVHNAKTKPREEQYNSTY |
| | | RVVSVLTVLHQDWLNGKEYKCKVSNKALPAPIEKTISKAKGQPREPQ |
| | | VYTLPPSRDELTKNQVSLTCLVKGFYPSDIAVEWESNGQPENNYKTTP |
| | | PVLDSDGSFFLYSKLTVDKSRWQQGNVFSCSVMHEALHNHYTQKSLS |
| | | LSPGK |
| 28 | synthetic sIL- | MAPRRARGCRTLGLPALLLLLLLRPPATRGITCPPPMSVEHADIWVKS |
| | 15Ra1pha-Fc | YSLYSRERYICNSGFKRKAGTSSLTECVLNKATNVAHWTTPSLKCIRD |
| | fusion protein | PALVHQRPAPPSTVTTAGVTPQPESLSPSGKEPAASSPSSNNTAATTAA |
| | huIL15sRa200- | IVPGSQLMPSKSPSTGTTEISSHESSHGTPSQTTAKNWELTASASHQPP |
| | Fc | GVYPQGPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSV |
| | | LTVLHQDWLNGKEYKCKVSNKALPAPIEKTISKAKGQPREPQVYTLP |
| | | PSRDELTKNQVSLTCLVKGFYPSDIAVEWESNGQPENNYKTTPPVLDS |
| | | DGSFFLYSKLTVDKSRWQQGNVFSCSVMHEALHNHYTQKSLSLSPGK |

PD-1 related sequences
BAP049-Clone-B HC

| | | |
|---|---|---|
| 29 | HCDR1(Kabat) | TYWMH |
| 30 | HCDR2(Kabat) | NIYPGTGGSNFDEKFKN |
| 31 | HCDR3(Kabat) | WTTGTGAY |
| 32 | HCDR1(Chothia) | GYTFTTY |
| 33 | HCDR2(Chothia) | YPGTGG |
| 34 | HCDR3(Chothia) | WTTGTGAY |
| 35 | VH | EVQLVQSGAEVKKPGESLRISCKGSGYTFTTYWMHWVRQATGQGLE |
| | | WMGNIYPGTGGSNFDEKFKNRVTITADKSTSTAYMELSSLRSEDTAV |
| | | YYCTRWTTGTGAYWGQGTTVTVSS |
| 36 | DNA VH | GAGGTGCAGCTGGTGCAGTCAGGCGCCGAAGTGAAGAAGCCCGGC |
| | | GAGTCACTGAGAATTAGCTGTAAAGGTTCAGGCTACACCTTCACTA |
| | | CCTACTGGATGCACTGGGTCCGCCAGGCTACCGGTCAAGGCCTCG |
| | | AGTGGATGGGTAATATCTACCCCGGCACCGGCGGCTCTAACTTCGA |
| | | CGAGAAGTTTAAGAATAGAGTGACTATCACCGCCGATAAGTCTAC |
| | | TAGCACCGCCTATATGGAACTGTCTAGCCTGAGATCAGAGGACAC |
| | | CGCCGTCTACTACTGCACTAGGTGGACTACCGGCACAGGCGCCTAC |
| | | TGGGGTCAAGGCACTACCGTGACCGTGTCTAGC |
| 37 | HC | EVQLVQSGAEVKKPGESLRISCKGSGYTFTTYWMHWVRQATGQGLE |
| | | WMGNIYPGTGGSNFDEKFKNRVTITADKSTSTAYMELSSLRSEDTAV |
| | | YYCTRWTTGTGAYWGQGTTVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGGP |
| | | SVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPDIAVEW |
| | | ESNGQPENNYKTTPPVLDSDGSFFLYSRLTVDKSRWQEGNVFSCSVM |
| | | HEALHNHYTQKSLSLSLG |
| 38 | DNA HC | GAGGTGCAGCTGGTGCAGTCAGGCGCCGAAGTGAAGAAGCCCGGC |
| | | GAGTCACTGAGAATTAGCTGTAAAGGTTCAGGCTACACCTTCACTA |
| | | CCTACTGGATGCACTGGGTCCGCCAGGCTACCGGTCAAGGCCTCG |
| | | AGTGGATGGGTAATATCTACCCCGGCACCGGCGGCTCTAACTTCGA |
| | | CGAGAAGTTTAAGAATAGAGTGACTATCACCGCCGATAAGTCTAC |
| | | TAGCACCGCCTATATGGAACTGTCTAGCCTGAGATCAGAGGACAC |
| | | CGCCGTCTACTACTGCACTAGGTGGACTACCGGCACAGGCGCCTAC |
| | | TGGGGTCAAGGCACTACCGTGACCGTGTCTAGCGCTAGCACTAAG |
| | | GGCCCGTCCGTGTTCCCCCTGGCACCTTGTAGCCGGAGCACTAGCG |
| | | AATCCACCGCTGCCCTCGGCTGCCTGGTCAAGGATTACTTCCCGGA |
| | | GCCCGTGACCGTGTCCTGGAACAGCGGAGCCCTGACCTCCGGAGT |
| | | GCACACCTTCCCCGCTGTGCTGCAGAGCTCCGGGCTGTACTCGCTG |
| | | TCGTCGGTGGTCACGGTGCCTTCATCTAGCCTGGGTACCAAGACCT |
| | | ACACTTGCAACGTGGACCACAAGCCTTCCAACACTAAGGTGGACA |
| | | AGCGCGTCGAATCGAAGTACGGCCCACCGTGCCCGCCTTGTCCCGC |
| | | GCCGGAGTTCCTCGGCGGTCCCTCGGTCTTTCTGTTCCCACCGAAG |
| | | CCCAAGGACACTTTGATGATTTCCCGCACCCCTGAAGTGACATGCG |
| | | TGGTCGTGGACGTGTCACAGGAAGATCCGGAGGTGCAGTTCAATT |
| | | GGTACGTGGATGGCGTCGAGGTGCACAACGCCAAAACCAAGCCGA |
| | | GGGAGGAGCAGTTCAACTCCACTTACCGCGTCGTGTCCGTGCTGAC |
| | | GGTGCTGCATCAGGACTGGCTGAACGGGAAGGAGTACAAGTGCAA |
| | | AGTGTCCAACAAGGGACTTCCTAGCTCAATCGAAAAGACCATCTC |
| | | GAAAGCCAAGGGACAGCCCCGGGAACCCCAAGTGTATACCCTGCC |
| | | ACCGAGCCAGGAAGAAATGACTAAGAACCAAGTCTCATTGACTTG |
| | | CCTTGTGAAGGGCTTCTACCCATCGGATATCGCCGTGGAATGGGAG |
| | | TCCAACGGCCAGCCGGAAAACAACTACAAGACCACCCCTCCGGTG |
| | | CTGGACTCAGACGGATCCTTCTTCCTCTACTCGCGGCTGACCGTGG |
| | | ATAAGAGCAGATGGCAGGAGGGAAATGTGTTCAGCTGTTCTGTGA |
| | | TGCATGAAGCCCTGCACAACCACTACACTCAGAAGTCCCTGTCCCT |
| | | CTCCCTGGGA |

BAP049-Clone-B LC

| | | |
|---|---|---|
| 39 | LCDR1(Kabat) | KSSQSLLDSGNQKNFLT |
| 40 | LCDR2(Kabat) | WASTRES |
| 41 | LCDR3(Kabat) | QNDYSYPYT |
| 42 | LCDR1(Chothia) | SQSLLDSGNQKNF |
| 43 | LCDR2(Chothia) | WAS |
| 44 | LCDR3(Chothia) | DYSYPY |
| 45 | VL | EIVLTQSPATLSLSPGERATLSCKSSQSLLDSGNQKNFLTWYQQKPGK |
| | | APKLLIYWASTRESGVPSRFSGSGSGTDFTFTISSLQPEDIATYYCQND |
| | | YSYPYTFGQGTKVEIK |
| 46 | DNA VL | GAGATCGTCCTGACTCAGTCACCCGCTACCCTGAGCCTGAGCCCTG |
| | | GCGAGCGGGCTACACTGAGCTGTAAATCTAGTCAGTCACTGCTGG |
| | | ATAGCGGTAATCAGAAGAACTTCCTGACCTGGTATCAGCAGAAGC |
| | | CCGGTAAAGCCCCTAAGCTGCTGATCTACTGGGCCTCTACTAGAGA |
| | | ATCAGGCGTGCCCTCTAGGTTTAGCGGTAGCGGTAGTGGCACCGA |
| | | CTTCACCTTCACTATCTCTAGCCTGCAGCCCGAGGATATCGCTACC |
| | | TACTACTGTCAGAACGACTATAGCTACCCCTACACCTTCGGTCAAG |
| | | GCACTAAGGTCGAGATTAAG |
| 47 | LC | EIVLTQSPATLSLSPGERATLSCKSSQSLLDSGNQKNFLTWYQQKPGK |
| | | APKLLIYWASTRESGVPSRFSGSGSGTDFTFTISSLQPEDIATYYCQND |
| | | YSYPYTFGQGTKVEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |
| 48 | DNA LC | GAGATCGTCCTGACTCAGTCACCCGCTACCCTGAGCCTGAGCCCTG |
| | | GCGAGCGGGCTACACTGAGCTGTAAATCTAGTCAGTCACTGCTGG |
| | | ATAGCGGTAATCAGAAGAACTTCCTGACCTGGTATCAGCAGAAGC |
| | | CCGGTAAAGCCCCTAAGCTGCTGATCTACTGGGCCTCTACTAGAGA |
| | | ATCAGGCGTGCCCTCTAGGTTTAGCGGTAGCGGTAGTGGCACCGA |
| | | CTTCACCTTCACTATCTCTAGCCTGCAGCCCGAGGATATCGCTACC |
| | | TACTACTGTCAGAACGACTATAGCTACCCCTACACCTTCGGTCAAG |
| | | GCACTAAGGTCGAGATTAAGCGTACGGTGGCCGCTCCCAGCGTGT |
| | | TCATCTTCCCCCCCAGCGACGAGCAGCTGAAGAGCGGCACCGCCA |
| | | GCGTGGTGTGCCTGCTGAACAACTTCTACCCCCGGGAGGCCAAGG |
| | | TGCAGTGGAAGGTGGACAACGCCCTGCAGAGCGGCAACAGCCAGG |
| | | AGAGCGTCACCGAGCAGGACAGCAAGGACTCCACCTACAGCCTGA |
| | | GCAGCACCCTGACCCTGAGCAAGGCCGACTACGAGAAGCATAAGG |
| | | TGTACGCCTGCGAGGTGACCCACCAGGGCCTGTCCAGCCCCGTGA |
| | | CCAAGAGCTTCAACAGGGGCGAGTGC |

BAP049-Clone-E HC

| | | |
|---|---|---|
| 49 | HCDR1(Kabat) | TYWMH |
| 50 | HCDR2(Kabat) | NIYPGTGGSNFDEKFKN |
| 51 | HCDR3(Kabat) | WTTGTGAY |
| 52 | HCDR1(Chothia) | GYTFTTY |
| 53 | HCDR2(Chothia) | YPGTGG |
| 54 | HCDR3(Chothia) | WTTGTGAY |
| 55 | VH | EVQLVQSGAEVKKPGESLRISCKGSGYTFTTYWMHWVRQATGQGLE |
| | | WMGNIYPGTGGSNFDEKFKNRVTITADKSTSTAYMELSSLRSEDTAV |
| | | YYCTRWTTGTGAYWGQGTTVTVSS |
| 56 | DNA VH | GAGGTGCAGCTGGTGCAGTCAGGCGCCGAAGTGAAGAAGCCCGGC |
| | | GAGTCACTGAGAATTAGCTGTAAAGGTTCAGGCTACACCTTCACTA |
| | | CCTACTGGATGCACTGGGTCCGCCAGGCTACCGGTCAAGGCCTCG |
| | | AGTGGATGGGTAATATCTACCCCGGCACCGGCGGCTCTAACTTCGA |
| | | CGAGAAGTTTAAGAATAGAGTGACTATCACCGCCGATAAGTCTAC |
| | | TAGCACCGCCTATATGGAACTGTCTAGCCTGAGATCAGAGGACAC |
| | | CGCCGTCTACTACTGCACTAGGTGGACTACCGGCACAGGCGCCTAC |
| | | TGGGGTCAAGGCACTACCGTGACCGTGTCTAGC |
| 57 | HC | EVQLVQSGAEVKKPGESLRISCKGSGYTFTTYWMHWVRQATGQGLE |
| | | WMGNIYPGTGGSNFDEKFKNRVTITADKSTSTAYMELSSLRSEDTAV |
| | | YYCTRWTTGTGAYWGQGTTVTVSSASTKGPSVFPLAPCSRSTSESTA |
| | | ALGCLVKDYFPEPVTVSWNSGALTSGVHTFPAVLQSSGLYSLSSVVT |
| | | VPSSSLGTKTYTCNVDHKPSNTKVDKRVESKYGPPCPPCPAPEFLGGP |
| | | SVFLFPPKPKDTLMISRTPEVTCVVVDVSQEDPEVQFNWYVDGVEVH |
| | | NAKTKPREEQFNSTYRVVSVLTVLHQDWLNGKEYKCKVSNKGLPSSI |
| | | EKTISKAKGQPREPQVYTLPPSQEEMTKNQVSLTCLVKGFYPSDIAVE |
| | | WESNGQPENNYKTTPPVLDSDGSFFLYSRLTVDKSRWQEGNVFSCSV |
| | | MHEALHNHYTQKSLSLSLG |
| 58 | DNA HC | GAGGTGCAGCTGGTGCAGTCAGGCGCCGAAGTGAAGAAGCCCGGC |
| | | GAGTCACTGAGAATTAGCTGTAAAGGTTCAGGCTACACCTTCACTA |
| | | CCTACTGGATGCACTGGGTCCGCCAGGCTACCGGTCAAGGCCTCG |
| | | AGTGGATGGGTAATATCTACCCCGGCACCGGCGGCTCTAACTTCGA |
| | | CGAGAAGTTTAAGAATAGAGTGACTATCACCGCCGATAAGTCTAC |
| | | TAGCACCGCCTATATGGAACTGTCTAGCCTGAGATCAGAGGACAC |
| | | CGCCGTCTACTACTGCACTAGGTGGACTACCGGCACAGGCGCCTAC |
| | | TGGGGTCAAGGCACTACCGTGACCGTGTCTAGCGCTAGCACTAAG |
| | | GGCCCGTCCGTGTTCCCCCTGGCACCTTGTAGCCGGAGCACTAGCG |
| | | AATCCACCGCTGCCCTCGGCTGCCTGGTCAAGGATTACTTCCCGGA |
| | | GCCCGTGACCGTGTCCTGGAACAGCGGAGCCCTGACCTCCGGAGT |
| | | GCACACCTTCCCCGCTGTGCTGCAGAGCTCCGGGCTGTACTCGCTG |
| | | TCGTCGGTGGTCACGGTGCCTTCATCTAGCCTGGGTACCAAGACCT |
| | | ACACTTGCAACGTGGACCACAAGCCTTCCAACACTAAGGTGGACA |
| | | AGCGCGTCGAATCGAAGTACGGCCCACCGTGCCCGCCTTGTCCCGC |
| | | GCCGGAGTTCCTCGGCGGTCCCTCGGTCTTTCTGTTCCCACCGAAG |
| | | CCCAAGGACACTTTGATGATTTCCCGCACCCCTGAAGTGACATGCG |
| | | TGGTCGTGGACGTGTCACAGGAAGATCCGGAGGTGCAGTTCAATT |
| | | GGTACGTGGATGGCGTCGAGGTGCACAACGCCAAAACCAAGCCGA |
| | | GGGAGGAGCAGTTCAACTCCACTTACCGCGTCGTGTCCGTGCTGAC |
| | | GGTGCTGCATCAGGACTGGCTGAACGGGAAGGAGTACAAGTGCAA |
| | | AGTGTCCAACAAGGGACTTCCTAGCTCAATCGAAAAGACCATCTC |
| | | GAAAGCCAAGGGACAGCCCCGGGAACCCCAAGTGTATACCCTGCC |
| | | ACCGAGCCAGGAAGAAATGACTAAGAACCAAGTCTCATTGACTTG |
| | | CCTTGTGAAGGGCTTCTACCCATCGGATATCGCCGTGGAATGGGAG |
| | | TCCAACGGCCAGCCGGAAAACAACTACAAGACCACCCCTCCGGTG |
| | | CTGGACTCAGACGGATCCTTCTTCCTCTACTCGCGGCTGACCGTGG |
| | | ATAAGAGCAGATGGCAGGAGGGAAATGTGTTCAGCTGTTCTGTGA |
| | | TGCATGAAGCCCTGCACAACCACTACACTCAGAAGTCCCTGTCCCT |
| | | CTCCCTGGGA |

BAP049-Clone-E LC

| | | |
|---|---|---|
| 59 | LCDR1(Kabat) | KSSQSLLDSGNQKNFLT |
| 60 | LCDR2(Kabat) | WASTRES |
| 61 | LCDR3(Kabat) | QNDYSYPYT |
| 62 | LCDR1(Chothia) | SQSLLDSGNQKNF |
| 63 | LCDR2(Chothia) | WAS |
| 64 | LCDR3(Chothia) | DYSYPY |
| 65 | VL | EIVLTQSPATLSLSPGERATLSCKSSQSLLDSGNQKNFLTWYQQKPGQ |
| | | APRLLIYWASTRESGVPSRFSGSGSGTDFTFTISSLEAEDAATYYCQND |
| | | YSYPYTFGQGTKVEIK |
| 66 | DNA VL | GAGATCGTCCTGACTCAGTCACCCGCTACCCTGAGCCTGAGCCCTG |
| | | GCGAGCGGGCTACACTGAGCTGTAAATCTAGTCAGTCACTGCTGG |
| | | ATAGCGGTAATCAGAAGAACTTCCTGACCTGGTATCAGCAGAAGC |
| | | CCGGTCAAGCCCCTAGACTGCTGATCTACTGGGCCTCTACTAGAGA |
| | | ATCAGGCGTGCCCTCTAGGTTTAGCGGTAGCGGTAGTGGCACCGA |
| | | CTTCACCTTCACTATCTCTAGCCTGGAAGCCGAGGACGCCGCTACC |
| | | TACTACTGTCAGAACGACTATAGCTACCCCTACACCTTCGGTCAAG |
| | | GCACTAAGGTCGAGATTAAG |
| 67 | LC | EIVLTQSPATLSLSPGERATLSCKSSQSLLDSGNQKNFLTWYQQKPGQ |
| | | APRLLIYWASTRESGVPSRFSGSGSGTDFTFTISSLEAEDAATYYCQND |
| | | YSYPYTFGQGTKVEIKRTVAAPSVFIFPPSDEQLKSGTASVVCLLNNFY |
| | | PREAKVQWKVDNALQSGNSQESVTEQDSKDSTYSLSSTLTLSKADYE |
| | | KHKVYACEVTHQGLSSPVTKSFNRGEC |
| 68 | DNA LC | GAGATCGTCCTGACTCAGTCACCCGCTACCCTGAGCCTGAGCCCTG |
| | | GCGAGCGGGCTACACTGAGCTGTAAATCTAGTCAGTCACTGCTGG |
| | | ATAGCGGTAATCAGAAGAACTTCCTGACCTGGTATCAGCAGAAGC |
| | | CCGGTCAAGCCCCTAGACTGCTGATCTACTGGGCCTCTACTAGAGA |
| | | ATCAGGCGTGCCCTCTAGGTTTAGCGGTAGCGGTAGTGGCACCGA |
| | | CTTCACCTTCACTATCTCTAGCCTGGAAGCCGAGGACGCCGCTACC |
| | | TACTACTGTCAGAACGACTATAGCTACCCCTACACCTTCGGTCAAG |
| | | GCACTAAGGTCGAGATTAAGCGTACGGTGGCCGCTCCCAGCGTGT |
| | | TCATCTTCCCCCCCAGCGACGAGCAGCTGAAGAGCGGCACCGCCA |
| | | GCGTGGTGTGCCTGCTGAACAACTTCTACCCCCGGGAGGCCAAGG |
| | | TGCAGTGGAAGGTGGACAACGCCCTGCAGAGCGGCAACAGCCAGG |
| | | AGAGCGTCACCGAGCAGGACAGCAAGGACTCCACCTACAGCCTGA |
| | | GCAGCACCCTGACCCTGAGCAAGGCCGACTACGAGAAGCATAAGG |
| | | TGTACGCCTGCGAGGTGACCCACCAGGGCCTGTCCAGCCCCGTGA |
| | | CCAAGAGCTTCAACAGGGGCGAGTGC |

BAP049-Clone-B HC

| | | |
|---|---|---|
| 69 | HCDR1(Kabat) | ACCTACTGGATGCAC |
| 70 | HCDR2(Kabat) | AATATCTACCCCGGCACCGGCGGCTCTAACTTCGACGAGAAGTTTA |
| | | AGAAT |
| 71 | HCDR3(Kabat) | TGGACTACCGGCACAGGCGCCTAC |
| 72 | HCDR1(Chothia) | GGCTACACCTTCACTACCTAC |
| 73 | HCDR2(Chothia) | TACCCCGGCACCGGCGGC |
| 74 | HCDR3(Chothia) | TGGACTACCGGCACAGGCGCCTAC |

BAP049-Clone-B LC

| | | |
|---|---|---|
| 75 | LCDR1(Kabat) | AAATCTAGTCAGTCACTGCTGGATAGCGGTAATCAGAAGAACTTC |
| | | CTGACC |
| 76 | LCDR2(Kabat) | TGGGCCTCTACTAGAGAATCA |
| 77 | LCDR3(Kabat) | CAGAACGACTATAGCTACCCCTACACC |
| 78 | LCDR1(Chothia) | AGTCAGTCACTGCTGGATAGCGGTAATCAGAAGAACTTC |
| 79 | LCDR2(Chothia) | TGGGCCTCT |
| 80 | LCDR3(Chothia) | GACTATAGCTACCCCTAC |

BAP049-Clone-E HC

| | | |
|---|---|---|
| 81 | HCDR1(Kabat) | ACCTACTGGATGCAC |
| 82 | HCDR2(Kabat) | AATATCTACCCCGGCACCGGCGGCTCTAACTTCGACGAGAAGTTTA |
| | | AGAAT |
| 83 | HCDR3(Kabat) | TGGACTACCGGCACAGGCGCCTAC |
| 84 | HCDR1(Chothia) | GGCTACACCTTCACTACCTAC |
| 85 | HCDR2(Chothia) | TACCCCGGCACCGGCGGC |
| 86 | HCDR3(Chothia) | TGGACTACCGGCACAGGCGCCTAC |

BAP049-Clone-E LC

| | | |
|---|---|---|
| 87 | LCDR1(Kabat) | AAATCTAGTCAGTCACTGCTGGATAGCGGTAATCAGAAGAACTTC |
| | | CTGACC |
| 88 | LCDR2(Kabat) | TGGGCCTCTACTAGAGAATCA |
| 89 | LCDR3(Kabat) | CAGAACGACTATAGCTACCCCTACACC |
| 90 | LCDR1(Chothia) | AGTCAGTCACTGCTGGATAGCGGTAATCAGAAGAACTTC |
| 91 | LCDR2(Chothia) | TGGGCCTCT |
| 92 | LCDR3(Chothia) | GACTATAGCTACCCCTAC |
| 93 | BAP049-Clone- | GYTFTTYWMH |
| | B/E HC | |
| | Combined | |
| | HCDR1 | |
| | Kabat/Chothia | |

Amino acid sequences of the heavy and light chain leader
sequences for humanized mabs
BAP049-Clone-B

| | | |
|---|---|---|
| 94 | HC | MAWVWTLPFLMAAAQSVQA |
| 95 | LC | MSVLTQVLALLLLWLTGTRC |

BAP049-Clone-E

| | | |
|---|---|---|
| 96 | HC | MAWVWTLPFLMAAAQSVQA |
| 97 | LC | MSVLTQVLALLLLWLTGTRC |

Constant region amino acid sequences of human IgG heavy
chains and human kappa light chain (EU Numbering)

| | | |
|---|---|---|
| 98 | IgG4 (S228P) | ASTKGPSVFP LAPCSRSTSE STAALGCLVK DYFPEPVTVS |
| | mutant constant | WNSGALTSGV HTFPAVLQSS GLYSLSSVVT VPSSSLGTKT |
| | region amino | YTCNVDHKPS NTKVDKRVES KYGPPCPPCP APEFLGGPSV |
| | acid sequence | FLFPPKPKDT LMISRTPEVT CVVVDVSQED PEVQFNWYVD |
| | | GVEVHNAKTK PREEQFNSTY RVVSVLTVLH QDWLNGKEYK |
| | | CKVSNKGLPS SIEKTISKAK GQPREPQVYT LPPSQEEMTK |
| | | NQVSLTCLVK GFYPSDIAVE WESNGQPENN YKTTPPVLDS |
| | | DGSFFLYSRL TVDKSRWQEG NVFSCSVMHE ALHNHYTQKS |
| | | LSLSLGK |
| 99 | IgG4 (S228P) | ASTKGPSVFP LAPCSRSTSE STAALGCLVK DYFPEPVTVS |
| | mutant constant | WNSGALTSGV HTFPAVLQSS GLYSLSSVVT VPSSSLGTKT |
| | region amino | YTCNVDHKPS NTKVDKRVES KYGPPCPPCP APEFLGGPSV |
| | acid sequence | FLFPPKPKDT LMISRTPEVT CVVVDVSQED PEVQFNWYVD |
| | lacing C-terminal | GVEVHNAKTK PREEQFNSTY RVVSVLTVLH QDWLNGKEYK |
| | lysine (K) | CKVSNKGLPS SIEKTISKAK GQPREPQVYT LPPSQEEMTK |
| | | NQVSLTCLVK GFYPSDIAVE WESNGQPENN YKTTPPVLDS |
| | | DGSFFLYSRL TVDKSRWQEG NVFSCSVMHE ALHNHYTQKS |
| | | LSLSLG |
| 100 | IgG1 wild type | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKRVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 101 | IgG1 (N297A) | ASTKGPSVFP LAPSSKSTSG GTAALGCLVK DYFPEPVTVS |
| | mutant constant | WNSGALTSGV HTFPAVLQSS GLYSLSSVVT VPSSSLGTQT |
| | region amino | YICNVNHKPS NTKVDKRVEP KSCDKTHTCP PCPAPELLGG |
| | acid sequence | PSVFLFPPKP KDTLMISRTP EVTCVVVDVS HEDPEVKFNW |
| | | YVDGVEVHNA KTKPREEQYA STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW |
| | | QQGNVFSCSV MHEALHNHYT QKSLSLSPGK |
| 102 | IgG1 (D265A, | ASTKGPSVFP LAPSSKSTSG GTAALGCLVK DYFPEPVTVS |
| | P329A) mutant | WNSGALTSGV HTFPAVLQSS GLYSLSSVVT VPSSSLGTQT |
| | constant region | YICNVNHKPS NTKVDKRVEP KSCDKTHTCP PCPAPELLGG |
| | amino acid | PSVFLFPPKP KDTLMISRTP EVTCVVVAVS HEDPEVKFNW |
| | sequence | YVDGVEVHNA KTKPREEQYN STYRVVSVLT VLHQDWLNGK |
| | | EYKCKVSNKA LAAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 103 | IgG1 (L234A, | ASTKGPSVFP LAPSSKSTSG GTAALGCLVK DYFPEPVTVS |
| | L235A) mutant | WNSGALTSGV HTFPAVLQSS GLYSLSSVVT VPSSSLGTQT |
| | constant region | YICNVNHKPS NTKVDKRVEP KSCDKTHTCP PCPAPEAAGG |
| | amino acid | PSVFLFPPKP KDTLMISRTP EVTCVVVDVS HEDPEVKFNW |
| | sequence | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLTC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 104 | Human kappa | RTVAAPSVFI FPPSDEQLKS GTASVVCLLN NFYPREAKVQ |
| | constant region | WKVDNALQSG NSQESVTEQD SKDSTYSLSS TLTLSKADYE |
| | amino acid | KHKVYACEVT HQGLSSPVTK SFNRGEC |
| | sequence | |

### EXAMPLES

### Example 1: Demonstration of an Additive/Synergistic Effect of a Combination of IL-15/II-15Ra Complex and and Anti-PD-1 Antibody Molecule Using Human PBMCs

This example utilizes an ex vivo Staphylococcal enterotoxin B (SEB) assay in the presence of titrated concentrations of a recombinant heterodimeric IL-15/soluble IL-15Ra complex (hetIL-15) and a fixed concentration of the anti-PD-1 antibody molecule PDR001 to determine an increase in the production of IL-2 on whole blood activated by SEB. Six parameters were tested:

(i) hetlL-15 alone

(ii) hetlL-15+hIgG4 (isotype control) at 0.5 μg/ml

(iii) hetlL-15+PDR001 at 0.5 μg/ml

(iv) PDR001 at 0.5 μg/ml alone

(v) SEB at 1 ng/ml alone

(vi) No SEB

Materials and Methods

Fresh T-cell culture media was prepared based on the IMDM media from Gibco (12440-053) with the following additional supplements: 10% Fetal Bovine Serum (Life Technologies Cat. No. 26140-079), 1% Sodium Pyruvate (Gibco, Cat. No. 11360-070), 1% L-Glutamine (Gibco, Cat. No. 25030-081), 1% HEPES (Gibco, Cat. No. 15630-080), 1% Pen-Strep (Gibco, Cat. No. 15140-122) and 1% MEM NAA (Gibco, Cat. No. 11140-050).

For the assay, PBMCs were isolated from whole blood of 4 human donors (E-012, E421, E444 and 1011) using Leucocep (Greiner Bio-one, Cat# 227-290). After a final wash, the cells were re-suspended in 5 ml of T-cell culture media. A single cell suspension was generated by straining the cells and a 1:20 dilution prepared in 1 ml T cell culture media. Cell counts were made using a Vi-Cell XR (Cell Viability Analyzer). Cells were diluted to 4x10⁶cells/m1 in T-cell culture media and 50 μl cells were added to each well of a 96-well flat bottom plate (Costar, Cat# 3596).

4×1 μg/ml hetIL-15 (conc: 1.627mg/ml; Clinical grade) was prepared in T-cell culture media and a 1:10 dose titration was performed with 6-point dose responses down the plate. 50 μl of titrated hetIL-15 was added to the appropriate plate well. 4×0.5 μg/ml of PDR001 or the isotype control hIgG4(S228P) was prepared in T-cell media. 50 μl of media alone or 2 μg/ml PDR001 (cone: 10 mg/ml; Clinical grade) or hIgG4(S228P) (conc: 3.63 mg/ml) prepared stock was added to the appropriate groups/wells. The plates were incubated for 1 hr in a tissue culture incubator. For the groups ‘combo same time’ hetIL-15 was added on the same day as PDR001. For the ‘combo sequential’ hetIL-15 was freshly prepared and added to the culture 72 hr post PDR001 addition.

4×1 ng/ml of SEB was prepared in fresh T-cell culture media by first diluting a SEB stock of 2.5 mg/ml to 25 μg/ml (1:100), which was then used to prepare 4 ng/ml stock. 50 μl of 4×SEB was added to the appropriate well to a final concentration of 1 ng/ml after the plates had been incubated for 1 hr.

Control groups were prepared including: no SEB (2 wells), media alone plus SEB (2 wells) and PDR001 at 0.5 μg/ml plus SEB (2 wells). The tested groups include hetIL-15 alone, hetIL-15+hIgG4(S228P) and hetIL-15+PDR001. All samples in the tested groups were run in triplicate.

The plates were incubated at 37° C. in 5% CO₂ for 4 days. On day 4, the plates were spun at 2000 rpm for 2 min. Approximately 120 μl cell supernatants were collected into 96-well polypropylene V-bottomed plates (Greiner Bio-one, Cat# 651261, Lot E150935P). The plates were sealed and frozen at −80° C. until assayed.

IL-2 measurement was performed using V-PLEX (MSD, Cat# K151QQD-4) according to the manufacturer's protocol. Samples were diluted to 1:5 in Diluent2 from the kit and run in quadruplicate. Data were analysed using the MSD analysis software.

Results

At a concentration of 1 μg/ml, hetIL-15 was observed to potentiate SEB induced IL-2 production in all 4 donors. However, this effect was potentiated further by the presence of PDR001, particularly when the combination was administered at the same time, and as such the combination of hetIL-15 and PDR001 further enhanced SEB induced IL-2 production over that observed for hetIL-15 alone. These results therefore demonstrate an additive or synergistic effect for the combination of hetIL-15 with PDR001 on IL-2 production from donor PBMCs.

### Example 2: A Phase1/1b Study of IL-15/IL-15Ra Complex Alone or in Combination with an Anti-PD-1 Antibody Molecule in Adults with Metastatic Cancer

This example describes a study to determine the safety, tolerability, dose-limiting toxicity (DLT) and maximum tolerated dose (MTD) of subcutaneous (SC) recombinant heterodimeric IL-15/soluble IL-15Ra complexes (hetIL-15) administered alone or in combination with the anti-PD-1 antibody molecule PDR001 to human patients with metastatic cancers. The patients have a confirmed diagnosis of cancer that is now metastatic and refractory to or inappropriate for standard medical treatment. The study may also: (i) evaluate the preliminary anti-tumor activity of hetIL-15 alone and in combination with PDR001; (ii) characterize the pharmacokinetic (PK) profile of hetIL-15 alone and in combination with PDR001; (iii) assess the immunogenicity of hetIL-15 alone and in combination with PDR001; and (iv) assess the pharmacodynamic (PD) effect of hetIL-15 alone and in combination with PDR001.

The human patients selected for inclusion in the study meet all of the following criteria:

a. Age≥18 years.
b. Patients have histologically confirmed solid tumor malignancy that is metastatic or unresectable and have progressed on at least 1 prior therapy and for which standard curative or palliative measures do not exist or are associated with minimal patient survival benefit (as defined by the patient and/or the study physicians). Inclusion of patients having tumors that can be safely biopsied is encouraged.

In addition, the human patients selected for inclusion in the study may also meet one or more, or all of the following criteria:

c. Patients must have a site of disease amenable to biopsy and be a candidate for tumor biopsy according to the treating institution's guidelines. Patients must be willing to undergo a new tumor biopsy at baseline and again during therapy on this study.
d. Patients must have evaluable or measurable disease, defined as at least one lesion that can be accurately measured in at least one dimension (longest diameter to be recorded for non-nodal lesions and short axis for nodal lesions) as ≥20 mm with conventional techniques or as ≥10 mm with a spiral computed tomography (CT) scan.
e. Patients must have recovered to ≤grade 1 NCI Common Terminology Criteria for Adverse Events (CTCAE) version 4.0.3 from toxicity of prior chemotherapy or biologic therapy administered more than 4 weeks earlier.
f. Patients on bisphosphonates for any cancer or on hormone therapy for prostate cancer may continue this therapy. However, patients with prostate cancer must have confirmed metastatic disease that has progressed despite hormonal therapy producing castrate levels of testosterone. (Castrate testosterone levels occur within hours after castration and within 2 to 3 weeks of a luteinizing hormone-releasing hormone agonist.)
g. Eastern Cooperative Oncology Group (ECOG) performance status ≤1.
h. Patients must have normal organ and marrow function as defined below:

leukocytes ≥3,000/μL
absolute neutrophil count (ANC) ≥1,500/μL and platelets ≥100,000/ μL
Hemoglobin >8.0 g/dL and total bilirubin within normal institutional limits
AST/ALT≤2.5×institutional upper limit of normal
creatinine <1.5×institutional upper limit of normal or creatinine clearance ≥60 mL/min/1.73 m2 for patients with serum creatinine levels >1.5×higher than institutional normal.
   i. DLCO/VA and FEV1≥50% of predicted on pulmonary function tests.
   j. Secondary (metastatic) CNS tumors are allowed provided that they are clinically stable for a period of 30 days prior to study entry and there is not a requirement of steroid or anti-convulsant therapy.

Patients that meet one or more, or all of the following criteria may not be selected as patients for the study:
a. Patients who have received any prior IL-15 treatment. No cytotoxic therapy, immunotherapy, radiotherapy, major surgery or antitumor vaccines within 4 weeks prior to enrollment. However patients will be allowed to have had prior anti-CTLA-4 or anti PD-1/PD-L1 or nitrosoureas or mitomycin C for more than 6 weeks prior to cycle 1, day 1 (C1D1).
b. Patients who have malignant disease, other than that being treated in this study. Exceptions to this exclusion include the following: malignancies that were treated curatively and have not recurred within 2 years prior to study treatment; completely resected basal cell and squamous cell skin cancers; and completely resected carcinoma in situ of any type.
c. Patients with primary brain cancers or active CNS metastases.
d. Patients with a history of allergic reactions attributed to compounds of similar chemical or biologic composition to hetlL-15.
e. Patients with uncontrolled intercurrent illness including, but not limited to, ongoing or active infection, cognitive impairment, active substance abuse, or psychiatric illness/social situations that, in the view of the study physicians would preclude safe treatment or the ability to give informed consent and limit compliance with study requirements.
f. Patients with impaired cardiac function or clinically significant cardiac disease, including any of the following: (a) clinically significant and/or uncontrolled heart disease such as congestive heart failure requiring treatment (NYHA grade ≥2), uncontrolled hypertension or clinically significant arrhythmia; (b) QTcF>470 msec on screening ECG or congenital long QT syndrome; (c) acute myocardial infarction or unstable angina pectoris <3 months prior to study entry.
g. Patient's inability or refusal to practice effective contraception during therapy or the presence of pregnancy or active breastfeeding.
h. Patients with documented HIV infection or positive serology, active bacterial infections, serologic or PCR evidence for active or chronic hepatitis B or hepatitis C.
i. Patients with a history of severe asthma or absolute requirement for chronic inhaled corticosteroid medications.
j. Patients with a history of autoimmune disease
k. Patients requiring chronic treatment with systemic steroid therapy ≥10 mg/day prednisone or similar, other than replacement dose steroids in the setting of adrenal insufficiency. Topical, inhaled, nasal and ophthalmic steroids are not prohibited.
l. Use of any vaccines against infectious diseases (e.g. Influenza, varicella, pneumococcus) is not permitted within 4 weeks of initiation of study treatment.
m. Use of hematopoietic colony-stimulating growth factors (e.g. G-CSF, GMCSF, M-CSF), 2 weeks prior to start of study drug is not permitted; however an erythroid stimulating agent is allowed as long as it was initiated at least 2 weeks prior to the first dose of study treatment and is maintained at a stable dose.

The MTD and/or Recommended Dose for Expansion (RDE) of single agent hetlL-15 will be determined in the phase I part of this open-label study. After the identification of the MTD and/or RDE of the single agent, a dose expansion part may be opened to further characterize the safety, PK, PD, and preliminary activity of the monotherapy.

The MTD and/or RDE of the combination of hetIL-15 with PDR001 will be determined in the phase Ib part of this study. Upon identification of the MTD or RDE, an expansion part may be opened to further characterize the safety, PK, PD, and preliminary activity of the combination. The expansion part will consist of two groups, patients with cancers that are historically resistant to anti-PD-1 therapy and patients with cancers that are historically sensitive to anti-PD-1 therapy (historically sensitive cancers include but are not limited to NSCLC, melanoma, and bladder).

Patients will continue to receive single agent hetIL-15 or in combination with PDR001 until disease progression or until meeting a stopping rule.

### Example 3: Dose Escalation Guidelines for Phase I

3.1 hetlL-15 Dosing

Patients will receive a total of 6 SC injections of hetlL-15 (3 times a week [MWF] for 2 weeks), followed by a 2-week break during each treatment cycle (28 days). Patients will be assigned to a dose level sequentially based on their order of entry into the study. Table 2 provides the provisional dose levels that will be evaluated. It is possible for additional and/or intermediate dose levels to be added during the course of the study. In addition, alternate dosing schedules of hetIL-15 may be evaluated, for example, administering hetIL-15 once or twice weekly during the first two weeks of the cycle. Cohorts may be added at any dose level below the MTD in order to better understand safety, PK, and/or PD.

3.2 Starting Dose Rationale for Single Agent hetlL-15

The starting dose of 0.25 μg/kg/SC injection of hetlL-15 was selected because it is 5 times lower than the lowest dose tested in macaques (1.27 μg/kg) and 50 times lower than the NOAEL dose (12.67 μg/kg). This starting dose provides a safety margin of at least 10-fold with interspecies adjustment based on differences in body surface area.

TABLE 2
Cohort Number and hetIL-15 Dose Levels

| | SC Dose of hetIL-15 |
|---|---|
| Patient Cohort | (μg/kg/day × 6 per cycle) |
| 1 | 0.25 |
| 2 | 0.5 |
| 3 | 1 |
| 4 | 2 |
| 5 | 4 |
| 6 | 8 |

3.3 Guidelines for Phase I Single Agent Dose escalation and Determination of the MTD/RDE

The phase I portion of this study will consist of escalating doses of single agent hetlL-15 to determine the MTD and/or RDE. The MTD is defined as the dose level below the dose where ≥2 of 3 or 6 patients experienced a DLT and will be limited to events occurring during the first treatment cycle (first 28 days). Patients must complete a minimum of 1 cycle of treatment with the minimum safety evaluation and drug exposure or have a DLT within the first cycle of treatment to be considered evaluable for dose escalation decisions. If a patient did not experience DLT and did not meet the minimum exposure criterion, he or she will not be evaluable for determination of the MTD and will be replaced.

New patients will not be enrolled and begin treatment in the next dose cohort until all patients treated at the previous dose level have reached Cycle 1, Day 28 of treatment and agreement between the investigators and Sponsor has been reached.

The trial will begin with single patient dosing cohorts and will switch to a standard 3+3 design when the first Grade 2 or higher AE is observed. The dose escalation scheme based on the 3+3 algorithm is presented in FIG. 2. Single patient cohorts will begin dosing at 0.25 μg/kg/injection and, in the absence of any significant≥Grade 2 clinical or laboratory treatment-emergent AEs or DLTs, dose escalation will proceed sequentially as shown in Table 2 unless the patient has an AE that requires expansion of the dosing cohort or has a DLT.

During the course of the dose-escalation, it is possible for additional cohorts of up to 6 patients to be enrolled at any planned or intermediate dose level below the next dose level determined by 3+3 or the MTD in order to better understand safety, PK, and/or PD. Although the DLT data observed in these additional cohorts cannot be taken into account in the 3+3 algorithm (see Table 3), the Sponsor and the investigators may decide after review of these data to overrule the 3+3 algorithm if they both agree that it is more appropriate to open the next cohort at a dose lower than the one determined by the 3+3 algorithm.

TABLE 3
Dose Escalation

| | | |
|---|---|---|
| | Number of Patients | |
| Sce- | with DLT at a | |
| nario | Given Dose Level | Escalation Decision Rule |

New cohort 3 patients at a new dose level

| | | |
|---|---|---|
| A | DLT = 0/3 | New cohort 3 patients at next higher dose |
| | | level or same dose level if highest dose |
| | | level. |
| B | DLT = 1/3 | New cohort 3 patients at the same dose |
| | | level (go to D or E below). |
| C | DLT > 1/3 | New cohort 3 patients at next lower dose |
| | | level or declare MTD at next lower dose |
| | | level if 6 patients already tested (never |
| | | re-escalate). |

From scenario B above

| | | |
|---|---|---|
| D | DLT = 1/6 | New cohort 3 patients at next higher dose |
| | | level or declare MTD otherwise. |
| E | DLT > 1/6 | New cohort 3 patients at next lower dose |
| | | level or declare MTD at next lower dose |
| | | level if 6 patients already tested (never |
| | | re-escalate). |

After the identification of the MTD/RDE, an expansion part may be opened to further characterize the safety of the monotherapy. Nine patients will be included in the safety expansion and they will be treated at the MTD/RDE under the same schedule (3 times a week [MWF] for 2 weeks), followed by a 2-week break during each treatment cycle (28 days). Based on toxicity profiles, alternate dosing schedules of hetIL-15 may be explored, for example, administering hetIL-15 once or twice weekly during the first two weeks of the cycle.

### Example 4: Dose Escalation Guidelines for Phase Ib

4.1 PDR001 and hetlL-15 Dosing

The Phase Ib dose escalation portion of the study will consist of a fixed dose (400 mg, IV infusion, Q4W) of PDR001 and escalating doses of hetIL-15 to evaluate safety, tolerability and determine the MTD and/or RDE of the combination to be used in expansion cohorts. On days when PDR001 and hetIL-15 are administered on the same day, PDR001 will be administered first and hetIL-15 will be administered after the PDR001 infusion has been completed.

4.2 Starting Dose Rationale for Combination of hetlL-15 with PDR001

PDR001 will be given at a fixed dose (400mg Q4W IV infusion). The starting dose of hetlL-15 in combination will be 1 μg/kg/dose. Doses of 0.25, 0.5 and 1 μg/kg/dose have been evaluated and no DLTs have been identified. The 2 μg/kg/dose is currently being investigated. The dose of hetlL-15 will be escalated to determine the MTD and/or RDE in the combination that will be used in the expansion groups. Escalation of dose levels of hetlL-15 will proceed as outlined in Table 2, starting with the 1 μg/kg/dose level. It is possible for additional and/or intermediate dose levels to be added during the course of the study. In addition, alternate dosing schedules of hetIL-15, may be evaluated, for example, administering hetlL-15 once or twice weekly during the first two weeks of the cycle. Cohorts may be added at any dose level below the MTD in order to better understand safety, PK, and/or PD. At no point will doses or frequency of hetlL-15 exceed single agent MTD when given in combination with PDR001.

PDR001 is being tested in an ongoing, multicenter, open-label study CPDR001X2101 with a phase I dose escalation part followed by a phase II part. Pharmacokinetic data, modeling of the exposure data, and safety data obtained from dose escalation support the use of either flat or mg/kg dosing. The expected PDR001 Ctrough concentrations are in line with observed steady state mean Ctrough concentrations for Pembrolizumab, which is approved with substantial efficacy in several cancer types. The recommended phase 2 dose of PDR001 was declared as 400 mg Q4W Immune-related toxicities do not appear to be dose-related, and thus a fixed dose of PDR001 at 400 mg Q4W with escalation doses of het IL-15 starting at a dose of 1 mg/kg dose (proven to be a safe dose with no DLTs) will be the starting dose.

### 4.3 Guidelines for Phase Ib Dose Escalation and Determination of the MTD/RDE

The same guidelines as the ones presented in Section 3.3 above and Table 3 will be used for the escalation of the dual combination except that the period of DLT observation is extended to 2 cycles (first 56 days). Therefore, the MTD will be defined as the highest dose level at which less than 2 out of 6 patients (<33%) experience DLT in Cycles 1 or 2 (first 56 days).The first patient enrolled at a dose level will complete a minimum of 2 weeks of treatment before considering enrollment of the next patients in the cohort. Resulting MTD must have been demonstrated in at least 6 patients, with ≤1 patient having experienced a DLT within 56 days of the first dose.

### Example 5: Dose Expansion Guidelines for Phase Ib

Once an RDE for the combination has been declared in the phase Ib dose escalation part, patients will be enrolled in the dose expansion part of the study and will be administered the RDE identified for the combination hetlL-15+PDR001.

The dose expansion part will consist of two different groups listed below. Enrollment to the group is independent of whether the patient is PD-1 or PD-L1 treatment naive or pre-treated:

Group 1: patients with historically anti-PD-1 resistant cancers

Group 2: patients with historically anti-PD-1 sensitive cancers Each group will enroll 20 patients.

### Example 6: Definition of Dose-Limiting Toxicity

Dose limiting toxicities are defined in Table 4. All toxicities will be graded according to the NCI CTCAE version 4.03. Dose limiting toxicity is defined as Grade 3 or 4 AEs assessed as related to hetlL-15 or PDR001 or the combination that occur during Cycle 1 (the first 28 days) from the initiation of the monotherapy hetIL-15 treatment or 56 days from the initiation of the combination treatment with the following exceptions outlined in Table 4. In addition, Table 4 also lists some G2 AEs considered as DLTs:

TABLE 4
Dose Limiting Toxicities

For the purpose of dose escalation and cohort expansion, DLT defined as follows:
Any Grade 4 AEs are DLTs with the exception of:
Neutropenia lasting ≤5 days that is not associated with fever or other clinical symptoms.
Lymphopenia or Leukopenia lasting less than 7 days
Electrolyte abnormalities that are not associated with clinical sequelae or deemed to be not
clinically significant and are corrected with appropriate management or supplementation
within 72 hours of the onset.
Any Grade 3 AEs are DLTs with the exception of:
Infusion reaction that resolves to ≤Grade 1 within 6 hours.
Nausea and vomiting that resolves within 2 days after starting optimal anti-emetic therapy.
Thrombocytopenia without significant bleeding.
Diarrhea that resolves within 2 days after starting optimal anti-diarrhea treatment.
Hypertension that resolves within 7 days of injection or infusion
Infection or fever in the absence of neutropenia that resolves within <5 days.
Rash or photosensitivity that resolves within 7 days after starting treatment.
Fatigue that resolves within 7 days.
Immune-related adverse events that resolve within 7 days after starting treatment with
corticosteroids.
Anorexia that resolves within 3 days of injection or infusion
Asymptomatic anemia or symptomatic anemia that can be corrected with transfusion
AST, ALT, alkaline phosphatase, or total or indirect bilirubin CTCAE grade 3 for ≤7 days
The following Grade 2 AEs are considered DLTs:
Newly emerging grade 2 total bilirubin with ≥CTCAE Grade 2 AST/ALT.
Pneumonitis that does not resolve within 7 days after starting treatment with
corticosteroids.
Eye pain that does not respond to topical therapy and does not improve to Grade 1 severity
within 2 weeks of the initiation of topical therapy OR requires systemic treatment.
Other clinically significant toxicities, including a single event or multiple occurrences of
the same event may be considered as DLTs.

### 6.1 Duration of Therapy

Patients who neither experience DLT nor have evidence of clinical deterioration suggestive of disease progression per irRC will proceed to a second cycle of treatment and undergo radiographic disease assessment on Cycle 3, Day 1 (±3 days).

Patients who have evidence of disease progression per immune-related response criteria (irRC) will discontinue the protocol treatment. Patients who have stable disease (SD) or have some evidence of a therapeutic response (defined as >15% decrease in sum of marker lesions and/or improvement or disappearance of some non-measurable lesions and/or >10% decrease in tumor markers) at their post-Cycle 2 response assessment can continue study treatment until disease progression per irRC.

### 6.2 Duration of Follow Up

Patients who receive only hetlL-15 will be followed for 30 days following the last dose of study medication. Patients who received the combination will be followed for 150 days following the last administration of PDR001. Patients removed from study for unacceptable AE(s) or development of IL-15/IL-15Rα antibodies will be followed until resolution or stabilization of the AE.

### Example 7: Clinical Experience with het-IL-15

As of Jun. 23, 2016, six patients have been treated with hetlL-15 on the single agent dose escalation part of this study. Patients received 0.25 μg/kg/dose (N=1), 0.5 μg/kg/dose (N=2), or 1.0 μg/kg/dose (N=3) three times a week during the first two weeks of each 28 day cycle. No DLTs have been identified to date. Enrollment is continuing to the 1.0 μg/kg/dose escalation cohort.

1. A method of treating a cancer in a subject, the method comprising administering to the subject:
(a) at least one initial dose of an interleukin-15 (IL-15)/IL-15 receptor alpha (IL-15Ra) complex followed by repeated or escalating doses of the IL-15/IL-15Ra complex; in combination with (b) an anti-PD-1 antibody molecule.

2. The method according to claim 1, wherein the initial dose of IL-15/IL-15Ra is between 0.5 and 2 μg/kg.

3. The method according to claim 2, wherein the initial dose of IL-15/IL-15Ra is 1 μg/kg.

4. The method according to claim 1, wherein the repeated dose of the IL-15/IL-15Ra complex is 1 μg/kg.

5. The method according to claim 1, wherein the escalating dose of the IL-15/IL-15Ra complex is two times that of a previous dose.

6. The method according to claim 1, wherein the initial dose of IL-15/IL-15Ra complex is 1 μg/kg followed escalating doses of the IL-15/IL-15Ra complex of 2, 4 and 8 μg/kg.

7. The method according to claim 1, wherein the dose of the IL-15/IL-15Ra complex is administered subcutaneously once a week for three weeks.

8. The method according to claim 1, wherein the dose of IL-15/IL-15Ra complex is administered subcutaneously three times a week for two weeks.

9. The method according to claim 1, wherein the IL-15/IL-15Ra complex is a heterodimeric complex of human IL-15 and human soluble IL-15Ra.

10. The method according to claim 9, wherein the human IL-15 comprises residues 49 to 162 of the amino acid sequence of SEQ ID NO: 1 and the human soluble IL-15Ra comprises the amino acid sequence of SEQ ID NO: 10.

11. The method according to claim 1, wherein the dose of the anti-PD-1 antibody molecule is administered as a flat dose.

12. The method according to claim 11, wherein the flat dose of the anti-PD-1 antibody molecule is a 300 mg or 400 mg.

13. The method according to claim 1, wherein the dose of the anti-PD-1 antibody molecule is administered intravenously once every four weeks.

14. The method according to claim 1, wherein the anti-PD-1 antibody molecule comprises: a heavy chain variable region (VH) comprising a VHCDR1 amino acid sequence of SEQ ID NO: 29; a VHCDR2 amino acid sequence of SEQ ID NO: 30; and a VHCDR3 amino acid sequence of SEQ ID NO: 31; and a light chain variable region (VL) comprising a VLCDR1 amino acid sequence of SEQ ID NO: 32, a VLCDR2 amino acid sequence of SEQ ID NO: 33, and a VLCDR3 amino acid sequence of SEQ ID NO: 34.

15. The method according to claim 1, wherein the anti-PD-1 antibody molecule comprises a VH comprising the amino acid sequence of SEQ ID NO: 35 and a VL comprising the amino acid sequence of SEQ ID NO: 45 or 65.

16. The method according to claim 1, wherein the anti-PD-1 antibody comprises a human heavy chain constant region selected from IgG1, IgG2, IgG3 or IgG4 and a light chain constant region selected from kappa or lambda.

17. The method according to claim 16, wherein the anti-PD-1 antibody comprises one or more of:
(a) a human IgG4 heavy chain constant region with a Serine to Proline mutation at position 108 of SEQ ID NO: 98 or 99 and a kappa light chain constant region;
(b) a human IgG1 heavy chain constant region with an Asparagine to Alanine mutation at position 180 of SEQ ID NO: 101 and a kappa light chain constant region;
(c) a human IgG1 heavy chain constant region with an Aspartate to Alanine mutation at position 148 and Proline to Alanine mutation at position 212 of SEQ ID NO: 102, and a kappa light chain constant region; or
(d) a human IgG1 heavy chain constant region with a Leucine to Alanine mutation at position 117 and Leucine to Alanine mutation at position 118 of SEQ ID NO: 103, and a kappa light chain constant region.

18. The method according to claim 1, wherein the anti-PD-1 antibody molecule comprises a heavy chain comprising the amino acid sequence of SEQ ID NO: 37 and a light chain comprising the amino acid sequence of SEQ ID NO: 47 or 67.
